(19) United States
(12) Patent Application Publication
(10) Pub. No.: US 2023/0287316 A1
(43) Pub. Date: Sep. 14, 2023

(54) INTER-ORGAN PLATFORM WITH TISSUE-SPECIFIC NICHES FOR A MICROPHYSIOLOGICAL SYSTEM ON A CHIP

(71) Applicant: THE TRUSTEES OF COLUMBIA UNIVERSITY IN THE CITY OF NEW YORK, NEW YORK, NY (US)

(72) Inventors: Gordana Vunjak-Novakovic, New York, NY (US); Andrea Califano, New York, NY (US); Peter Sims, Ardsley, NY (US); Alan Chramiec, Brooklyn, NY (US); Ece Ozturk, New York, NY (US); Kacey Ronaldson, New York, NY (US); Keith Yeager, Jersey City, NJ (US); Diogo Teles, New York, NY (US)

(21) Appl. No.: 17/992,779

(22) Filed: Nov. 22, 2022

Related U.S. Application Data
(63) Continuation of application No. PCT/US2021/033833, filed on May 24, 2021.
(60) Provisional application No. 63/030,660, filed on May 27, 2020.
(60) Provisional application No. 63/028,945, filed on May 22, 2020.

(51) Int. Cl. C12M 3/00; C12M 1/42; C12M 1/12; C12N 5/09

(52) U.S. Cl. CPC C12M 21/08; C12M 23/44; C12M 25/06; C12M 35/08; C12N 5/0693

(57) ABSTRACT

Disclosed are systems and methods for culturing systemic bioengineered tumor models, including osteosarcoma cells or breast adenocarcinoma cells derived from patients. The model includes the tumor cells on host tissues such as bone, liver, lung, and heart tissue, wherein the bone, liver, lung, and heart tissue are separated by endothelial barriers. Beneath the endothelial barriers the tissues are connected via circulation containing secreted factors and cells (e.g., tumor/immune cells).

### RELATED APPLICATIONS

This application is a continuation of PCT/US2021/033833, filed May 24, 2021 which claims the benefit of U.S. Provisional Application Nos. 63/028,945, filed May 22, 2020 and 63/030,660, filed May 27, 2020, the contents of each of which are incorporated herein in their entirety.

### TECHNICAL FIELD

This disclosure relates to a modular microphysiological system including two or more wells having its own microenvironment or tissue-specific niche and bioengineered human tumors cultured in the integrated modular microphysiological system.

### GOVERNMENT FUNDING

This invention was made with government support under grants EB025765 and EB027062, and awarded by the National Institutes of Health. The government has certain rights in the invention.

### BACKGROUND

Current projections show that 40% of men and women can be diagnosed with cancer within their lifetime. Moreover, despite major advances in diagnosis and therapy, many patients do not respond or relapse after treatment. For example, cancer drugs, such as endostatin, have yielded promising results in mice, such as full tumor elimination when used alone, to subsequently show only minimal results in human patients. On the other hand, tamoxifen, a selective estrogen-receptor modulator, has been successfully used to treat breast cancer for years. However, if its predisposition to cause liver tumor in rats had been discovered in preclinical tests, the drug would have been eliminated during developmental testing. Other drugs have passed preclinical trials and then withdrawn, due to the side effects detected only during clinical trials or even after entering the market and being used by large numbers of patients. This is particularly true for cardiac side effects, as successful preclinical and clinical screening still allowed cardiotoxic drugs to enter the market. Rofecoxib, a COX-2 inhibitor used as an analgesic and antiinflammatory drug, was approved by the FDA in 1999 and then removed from market in 2004 because of side effects not seen in preclinical and clinical trials. Unfortunately, by that time, the drug had already caused an estimated 140,000 heart attacks associated with 60,000 deaths. Results like these illustrate the need for more predictive models of drug safety and efficacy, which would enable thorough testing of cardiac side effects. While regulatory changes have prevented drugs causing lethal arrhythmia from reaching the market, the current screening models are often oversensitive to proarrythymic side effects and result in elimination of numerous drugs. To date, as high as 60% of new drugs test positive for proarrhythmic events, based on assessing the rapid component of the delayed rectifier potassium current (IKr) for its blocking liability. The false positives are responsible for preventing the potentially lifesaving compounds from reaching the market.

Meanwhile, metastatic progression - a major determinant of poor outcome in treatment—is difficult to study both in patients and in existing tumor models. Cell lines present significant mutational and transcriptional drifts, abnormal ploidy, and loss of heterogeneity, compared to tumor cells in patients. These differences are compounded by poor recapitulation of the original pathophysiological milieu and growth conditions, including 2D vs. 3D-growth, lack of extracellular matrix (ECM), lack of surrounding cells (stromal, vascular, immune), as well as lack of molecular signals and physical constraints. Indeed, tumorigenesis strongly depends on cancer cell interactions with the environment. Animal models provide a more physiological environment, but are laborious, expensive, and do not support fine-grain control of exogenous factors. Over the last decade, bioengineering has entered the field of cancer research, by introducing more physiologically relevant 3- dimensional (3D) models of cancer growth: tumor spheroids and organoids, vascularization and scaffolds. These models fail to recapitulate critical aspects of human pathophysiology, such as the interactions of the tumor compartment with other cells (paracrine interactions) and organs (endocrine interactions). In addition, no currently available bioengineered model is able to model the full complexity of tumor progression, from primary tumor growth to intravasation in the blood stream, and seeding a distal organ site. Indeed, a human tissue model that would accurately model these aspects of metastatic progression would be transformative to cancer research. There is, thus, a need to understand human biology and system wide pathology - so we can predict which drugs can work for which patients before clinical trials. While this need is partly addressed using in vitro and animal models for most therapeutic areas, their lack of utility in modeling systemic diseases significantly prohibits the development of drugs for many diseases affecting more than one tissue system.

What is needed is a preclinical model that could more accurately predict both the efficacy and the safety of new drugs in humans that could enable more reliable drugs to progress through the developmental pipeline. While the development of human induced pluripotent stem (iPS) cells provides a human cell source for preclinical testing, the relative immaturity and the lack of biological fidelity limit their use.

### SUMMARY

Generally, in one aspect of the disclosed subject matter provides a highly advanced “cancer patient on a chip” model that uses vascular perfusion to physiologically integrate bioengineered human tumors with the target tissues to which they preferentially metastasize (lung, liver, bone), all derived from same-patient cells. In one aspect the subject matter provides to a platform with bioengineered human tissue niches linked by vascular perfusion can recapitulate effects and biomarkers of multi-organ drug toxicities. The platform allows systemic level tissue communication, maintains the engineered tissue phenotypes, and can thereby facilitate clinical translation. The platform provides a human, systemic model of patient specific disease “on-a-chip” or “in-a-dish” - benefiting drug developers, patients, clinicians, and the healthcare economy.

The disclosure provides a method for co-culturing two or more differentiated cell phenotypes, the method comprising
placing each differentiated cell phenotype in a well of an integrated modular microphysiological system comprising two or more wells configured for culturing a tissue, each of said wells comprising a layer of endothelial cells which forms an endothelial barrier at the bottom of the well; and a vascular network comprising at least one channel, wherein each of said endothelial barriers in in fluid contact with at least one of said at least one channel; and
circulating a culture medium through the vascular network, wherein the culture medium perfuses through each of the endothelial barriers into each of the wells each containing a differentiated phenotype and the endothelial barriers prevent secreted cytokines and cells from circulating out of each of the wells into other wells. The circulating medium may contain circulating cells (e.g, cancer and immune).

Embodiments include the following, alone or in any combination:

The method wherein the differentiated phenotypes are selected from the group consisting of heart, liver, bone, lung and skin phenotypes.

The method wherein the differentiated phenotypes are co-cultured for up to 4 weeks.

The method further comprising assessing the fidelity of the co-cultured phenotypes by proteomic analysis.

The method further comprising circulating a test compound through the vascular network.

The method wherein the test compound comprises a pharmaceutical agent.

The method wherein the pharmaceutical agent comprises dofetilide, epinephrine or doxorubicin

In another aspect, the present disclosure is directed to a composition comprising bioengineered tumor. The bioengineered tumor comprises osteosarcoma cells derived from a patient. In another embodiment, the bioengineered tumor comprises breast adenocarcinoma cells derived from a patient.

The present disclosure is also directed to a composition comprising at least one host tissue.

The present disclosure is also directed to a system of bioengineered tumor and at least one host tissue.

In an embodiment, the at least one host tissue comprises bone, liver, lung, and heart tissue, wherein the bone, liver, lung, and heart tissue are separated by an endothelial barrier. The endothelial barrier is exposed to flow/shear stress.

### BRIEF DESCRIPTION OF THE DRAWINGS

A detailed description of various aspects, features and embodiments of the subject matter described herein is provide with reference to the accompanying drawings, which are briefly described below. The drawings are illustrative and are not necessarily drawn to scale, with some components being exaggerated for clarity. The drawings illustrate various aspects and features of the present subject matter and may illustrate one or more embodiment(s) or example(s) of the present subject matter in whole or in part. Together with the description, the drawings serve to explain the principles of the disclosed subject matter.

FIGS. 1A-1E show aspects of the system that enable maintenance of tissue-specific niches while allowing for tissue cross-talk, in accordance with an exemplary embodiment of the disclosed subject matter.

FIGS. 2A-2I show aspects of the system configurability and modularity, in accordance with an exemplary embodiment of the disclosed subject matter.

FIGS. 3A-3G show various perspective views of the modular system, in accordance with an exemplary embodiment of the disclosed subject matter.

FIGS. 4A-4D shows a modular system having removable chambers in accordance with an exemplary embodiment of the disclosed subject matter.

FIG. 5 shows the modular system of FIGS. 4A-4D having an onboard pump in accordance with an exemplary embodiment of the disclosed subject matter.

FIGS. 6A-6L show aspects of the cardiac maturation as a result of electromechanical stimulation at an increasing intensity, in accordance with an exemplary embodiment of the disclosed subject matter.

FIGS. 7A-7I show aspects of the myocardin regulation of cardiac maturity to enable recapitulation of clinically observed drug

FIGS. 8A-8G shows aspects of the maintenance of structural, functional, and molecular phenotypes of engineered tissues over 4-week culture, in accordance with an exemplary embodiment of the disclosed subject matter.

FIGS. 9A-9F shows aspects of the proteomic breakdown for all four tissues, in accordance with an exemplary embodiment of the disclosed subject matter.

FIGS. 10A-10D shows aspects of the proteomic breakdown for cardiac tissue, in accordance with an exemplary embodiment of the disclosed subject matter

FIGS. 11A-11E shows aspects of proteomic analysis that confirm biological fidelity of InterOrgan platform in comparison to isolated cultures and adult human tissues, in accordance with an exemplary embodiment of the disclosed subject matter.

FIGS. 12A-12D shows aspects of the drug distribution in the platform measured by LC-MS quantification of drug concentrations with functional matching, in accordance with an exemplary embodiment of the disclosed subject matter.

FIGS. 13A-13M shows aspects of the platform revealing predictive off-target responses to doxorubicin, in accordance with an exemplary embodiment of the disclosed subject matter.

FIGS. 14A-14F shows aspects of the miRNA data for doxorubicin cardiotoxicity matched to patient data, in accordance with an exemplary embodiment of the disclosed subject matter.

FIGS. 15A-15C shows aspects of the doxorubicin-induced cardiotoxicity matched to clinical benchmarks via gene set enrichment analysis, in accordance with an exemplary embodiment of the disclosed subject matter.

FIGS. 16A-16E shows aspects of bioengineering of matured canonic target tissues for breast cancer metastasis, according to exemplary embodiments of the disclosed subject matter.

FIGS. 17A-C show aspects of vascular development prior to osteogenesis, according to exemplary embodiments of the disclosed subject matter.

FIGS. 18A-18B shows aspects of bioengineered breast tumor tissue, according to exemplary embodiments of the disclosed subject matter.

FIGS. 19A-19C shows aspects of bioengineered lung tissue, according to exemplary embodiments of the disclosed subject matter.

FIGS. 20A-20F shows aspects of breast tumor cells into the human bone perivascular niche-on-a-chip, according to exemplary embodiments of the disclosed subject matter.

FIGS. 21A-21F shows aspects of the endothelial barrier, according to exemplary embodiments of the disclosed subject matter.

FIGS. 22A-22F shows examples for the application of large-scale single-cell RNA-Seq to surgical specimens of human tumors, according to exemplary embodiments of the disclosed subject matter.

FIGS. 23A-23G show aspects of breast cancer bioengineering, according to exemplary embodiments of the disclosed subject matter.

FIG. 24 shows process steps according to exemplary embodiments of the disclosed subject matter.

FIGS. 25A-25D shows additional aspects of the system according to exemplary embodiments of the disclosed subject matter.

FIGS. 26A-26F shows aspects of concentration profiles of a hydrophobic small-molecule tracer and linsitinib circulation within the system.

FIGS. 27A-27B shows views of a microscope system, according to exemplary embodiments of the disclosed subject matter.

FIGS. 28A-28G shows aspects of the development and validation of the engineered human Ewing sarcoma (ES) bone tumor and human cardiac tissue, according to exemplary embodiments of the disclosed subject matter.

FIGS. 29A-29D shows aspects of immunohistochemical (IHC) staining of engineered ES tumors with transduced GFP-luciferase positive cancer cells, according to exemplary embodiments of the disclosed subject matter.

FIGS. 30A-30D shows aspects of valuation of the dose-dependent effects of linsitinib concentration on metastatic and non-metastatic Ewing sarcoma cell line monolayers, according to exemplary embodiments of the disclosed subject matter.

FIGS. 31A-31E shows aspects of the evaluation of the dose-dependent effects of doxorubicin and linsitinib on engineered non-metastatic and metastatic ES bone tumors, according to exemplary embodiments of the disclosed subject matter.

FIGS. 32A-32D shows aspects of responses of human engineered bone ES tumors and cardiac tissues to linsitinib in isolated platform chambers, according to exemplary embodiments of the disclosed subject matter.

FIGS. 33A-33B shows the effects of linsitinib on ES cells and osteoblasts within the engineered ES bone tumors, according to exemplary embodiments of the disclosed subject matter.

FIGS. 34A-34G summarizes the evaluation of the effects of linsitinib on the non-metastatic engineered ES bone tumor and cardiac tissues in the integrated platform, according to exemplary embodiments of the disclosed subject matter.

FIGS. 35A-35E shows aspects of responses of human engineered bone ES tumors and cardiac tissues to linsitinib in the integrated platform with microfluidic perfusion, according to exemplary embodiments of the disclosed subject matter.

FIG. 36 shows a configurable platform in which the tissues are connected by vascular perfusion with endothelial barrier and circulating cancer and immune cells, according to exemplary embodiments of the disclosed subject matter.

FIGS. 37A-37C shows aspects of bioengineering of stable and tissue-specific endothelium with tunable permeability, according to exemplary embodiments of the disclosed subject matter.

FIGS. 38A-38E shows selective extravasation of breast cancer cells into the tissue compartments, according to exemplary embodiments of the disclosed subject matter.

FIG. 39 shows a bone marrow model overview comprising iPSC-derived stromal cell types include osteoblasts, mesenchymal stem/stromal cells, endothelial cells & hematopoietic cell types include iPSC-derive HSCs, myeloid progenitors, lymphoid progenitors, mature macrophages/monocytes, mature B cells, immature T cells, megakaryocytes.

FIGS. 40A-D show validation of model after 1 week of culture. FIG. 40A shows total cell counts with various sub-specifications of hematopoietic cells. FIG. 40B shows percentage of cells attached to the bone marrow niche compartment. FIG. 40C shows fold expansion of CD45+ and CD34+ cells with minimal cytokines. FIG. 40D shows colony-forming capacity of isolated cells post-culture.

FIG. 41 shows bone marrow morphology with stains for osteoblasts (BSP) and hematopoietic cells (CD45).

FIG. 42 shows application of bone marrow niche to modeling radiotoxicity of human tissue in vitro. Morphological features demonstrated in top panels 2 weeks after exposure to 0G, 2G, 4G, and 6G of radiation in pentachrome staining, as well as expansion/progenitor differentiation capacity after 2 weeks post-radiation exposure of hematopoietic cells.

FIG. 43 shows bone marrow response to radiation damage.

FIG. 44 shows effects of radiation on hematopoietic function.

### DETAILED DESCRIPTION

It is to be understood that both the foregoing general description and the following detailed description are exemplary and explanatory only, and are not restrictive of the invention, as claimed. In this description, the use of the singular includes the plural, the word “a” or “an” means “at least one,” and the use of “or” means “and/or,” unless specifically stated otherwise. Furthermore, the use of the term “including,” as well as other forms, such as “includes” and “included” is not limiting. Also, terms such as “element” or “component” encompass both elements or components comprising one unit and elements or components that comprise more than one unit unless specifically stated otherwise. The use of the term “or” in the claims and the present disclosure is used to mean “and/or” unless explicitly indicated to refer to alternatives only or the alternatives are mutually exclusive.

Use of the term “about”, when used with a numerical value, is intended to include +/-10%. For example, if a number of amino acids is identified as about 200, this would include 180 to 220 (plus or minus 10%).

The terms “patient,” “individual,” and “subject” are used interchangeably herein, and refer to a mammalian subj ect to be treated, with human patients being preferred. In some cases, the methods of the invention find use in experimental animals, in veterinary application, and in the development of animal models for disease, including, but not limited to, rodents including mice, rats, and hamsters, and primates.

Bioengineered tissue systems offer a new paradigm for modeling human pathophysiology and testing drug efficacy and safety. However, establishing physiological communication between multiple tissues while also preserving their individual phenotypes is a major challenge due to the conflicting requirements for maintaining each tissue-specific regulatory niche. We describe herein a biomimetic InterOrgan bioreactor system in which each tissue can be cultured in its own specific optimized environment within the system. Each environment is a specific tissue niche that is separated by a selectively permeable endothelial barrier from recirculating flow containing circulating monocytes. The specific tissues, however, are linked by vascular perfusion in the system. The tissues maintain their molecular, structural and functional phenotypes over four weeks of culture. The system, thus, defines a plurality of bioengineered human tissue niches that are linked by vascular perfusion to enable recapitulated effects and biomarkers of multi-organ drug toxicities. In the system described herein, multiple tissues can be connected without sacrificing their individual biological fidelity. The tissues cultured in this manner recapitulate the clinically observed multi-organ toxicity of doxorubicin, and allow identification of miRNA biomarkers of cardiotoxicity. Overall, the bioreactor system (also referred to herein as InterOrgan platform) allows systemic level tissue communication, maintains the engineered tissue phenotypes, and can thereby facilitate clinical translation.

Other approaches are unable to maintain individual tissue health and functionality/gene expression/proteomics/drug responses when connecting multiple tissues together. For example, current methods rely either on transferring supernatant between tissues or the use of “common media” containing the factors required by all tissues. In contrast, the organs in our body maintain their own environments while being linked by vasculature lined with selectively permeable endothelium.

The system enables: 1) maintaining biological fidelity of tissues (engineered or patient derived) over long and short term culture times, 2) maintaining tissues in their niche while allowing communication via secreted factors, 3) allow cells and biologics (secreted factors, exosomes) to move between compartments, 4) allow circulating cells to preferentially extravasate from the vasculature into tissues as biologically appropriate (metastasis to expected tissue site, immune infiltration into damaged tissues, cell therapy), 5) drug studies in the integrated platform that has more clinical relevance as an integrated system versus the sum of their parts, qualifying its use as a clinical human correlate for disease modeling and drug testing.

All of the above can be done in a patient specific manner, using patient derived cells in healthy and diseased states with ability to also include genetically engineered cells of induced disease or health.

The system’s versatility lies in connecting millimeter-sized engineered tissues via vascular perfusion, with each tissue in its own optimized microenvironment, while still allowing for natural, selectively permeable cytokine and cellular cross-talk across the vascular endothelium separating the tissue and perfusion spaces. In one aspect, the system is modular and various different tissues, organoids, or patient biopsies can be combined as desired. In one aspect, the system of the present disclosure enables multiple tissues to be plugged directly into a platform of the system configured to receive the tissues, to enable multi-tissue studies in a way that tissues are connected through a vascular network but maintain their own tissue-specific niche for enhanced functionality, transcriptomics, and clinically relevant drug responses. Additionally, the ability to include human immune interactions and cellular movement from one compartment to another, as within the human body, is a major innovation facilitating the platforms translational impact. I

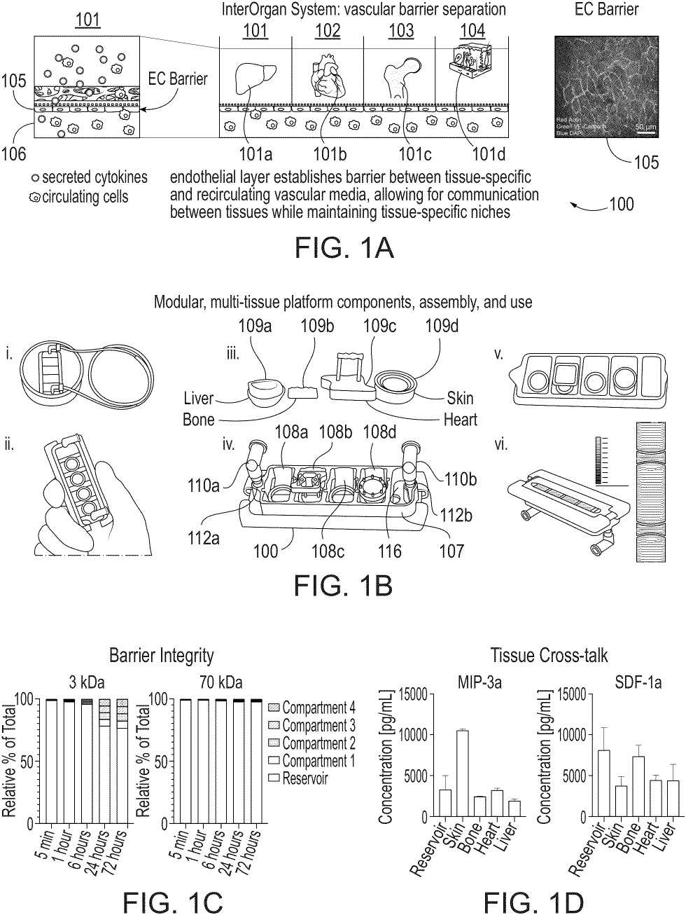

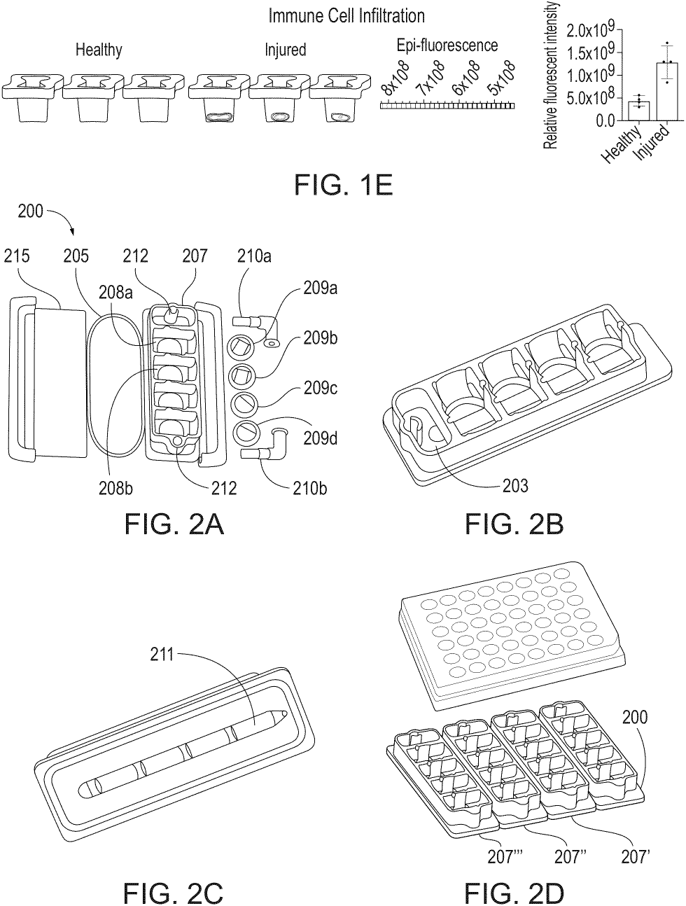

Referring to FIGS. 1A-D, in one embodiment, the system defines tissue-specific niches 101, 102, 103, 104 while also allowing for tissue cross-talk between these niches. In FIG. 1A, the vascular barrier 105 (shown as EC Barrier) retains one or more tissue-specific niches (101, 102, 103, 104) creating an environment for different tissues, such as liver 101a, heart 102a, bone 103a, skin 104a. The tissues (101a, 102a, 103a and 104a) can communicate via recirculating vascular media 106, i.e., cross-talk, disposed and flowing proximate the selectively permeable EC barrier 105. The selectively permeable barrier 105 in some embodiments is an endothelial layer that establishes a barrier between tissue-specific niches 101, 102, 103, and 104 defined by platform 107 (discussed below) and recirculating vascular media 106. As shown secreted cytokines and circulating cells can pass through the EC barrier to permit tissue cross talk. The endothelial barrier is the primary mediator of transport between each niche and the recirculating flow. It is supported by a relatively large-porosity membrane (preferably about ~20um pore size, or 8 to 60um potentially). The permeability of the endothelial barrier can change in response to environmental conditions, primarily medium flowrate or biochemical composition. The barrier also selectively transports molecules and cells and allows for a more physiological drug transport (e.g., PK/PD).

Tissue cross-talk between the liver 101a, heart 101b, bone 101c and skin 101d is depicted in FIG. 1D. Referring to FIG. 1D, secreted growth factors and cytokines (MIP-3a, SDF-1a) reached other tissue compartments (n=5) demonstrating that the bioreactor platform permits cross-talk between tissues in different, separated tissue- niches.

In another aspect, the bioreactor system, as shown in FIG. 1B, is a configurable, “plug-and-play” modular system comprising a platform 107 engaged to housing. The platform 107 comprises two or more wells or chambers 108a-108d. The chambers 108a-108d are configured to receive a “plug” or insertable device 109a to 109d, each of which contain a tissue, e.g., 101a-101d. Tissues, for example, may be liver, bone, skin and heart (See 1B iii). One of ordinary skill in the art would appreciate, however, that other tissues or organoids may be used. As depicted, the insertable devices 109a-109d are configured to plug into respective chambers 108a-108d, and thereby define separated, tissue-specific niches 101, 102, 103 and 104 of the system.

In one embodiment, as shown in FIG. 1B v and 1B ii, the platform 107 is a rectangular shaped base having a plurality of chambers 108a-108d defined by sidewalls. Each chamber 108a-108d has a bottom surface and sidewalls that separate the tissue-specific niche environment from each other. The bottom surfaces of the chambers include an opening to allow selective flow of vascular media 106 into the tissue specific niche environments. The opening can be defined by a channel 110 disposed between the bottom surface of the platform 107 and a plate (not shown) secured to the platform by housing member and O-ring, for example. Vascular media enters and exits the bioreactor system through tubular microfluidic connectors 110a and 110b that matingly engage ports 112a and 112b disposed on the bioreactor platform base 107. In one embodiment, the tubular microfluidic connectors 110a and 110b have first and second legs forming a substantially right angle, but other angles can be implemented so long as the media can flow through the tubular microfluidic connectors and into channel 110. The channel 110 is best viewed in FIG. 1B vi. The channel opening 111 permits vascular media 106 to selectively flow through vascular barrier 105 into the tissue specific niches defined by the tissue culture chambers (101, 102, 103 and 104) and insertable devices (108, a, 108b, 108c, 108d) to allow cross-talk between the tissues contained in the chambers, as described above. Cross-talk is essential for an integrated multi-tissue system. By allowing cross-talk, each tissue can respond to signals excreted by other organs/tissues. In complete isolation, the factors excreted by other tissues are never seen. The other extreme, complete integration in a shared medium (full mixing), the ideal niche of an individual tissues cannot be maintained. It is the endothelial barrier that prevents complete mixing, thus maintaining individual tissue niches, while mediating what factors can traverse the barrier in either direction. Thus, cross-talk also enables inter-tissue communication via exosomes, cells, and secreted factors. The endothelial barrier allows crosstalk to occur without having full mixing of the tissue-specific medias, enabling each tissue to be cultured in the optimal media (with supporting growth factors and small miolecules) while still permitting tissue-tissue crosstalk as you see in the body.

Referring now to FIG. 1C, barrier integrity of the vascular barrier 105 is depicted. Barrier function over 72 hours was tested by tracking tagged dextran molecules (n=4) having 3 kDa and 70 kDa. As shown in FIG. 1C, shows an experiment to characterize the permeability of the barrier. Smaller molecules (3 kDa) can enter, while larger molecules (70 kDa) cannot.

In FIG. 1E, circulating monocytes extravasated through the vascular barrier to injured cardiac tissues, as opposed to healthy adjacent tissue (n=4). ** p <0.01. The endothelieum allows cells to cross into the tissue space (immune and cancer). Many immune cells within the body only enter tissues upon injury to promote repair, and the InterOrgan platform enables replication of this process. The endothelial cells actively transport the monocytes across the barrier in response to injury. These cells would normally not be able to traverse the barrier solely based on their size (much larger than the molecules above). This shows that, even though the barrier remained intact, the monocytes traversed the barrier. There was an active effort by the endothelial cells to squeeze/pull the cells through the barrier.

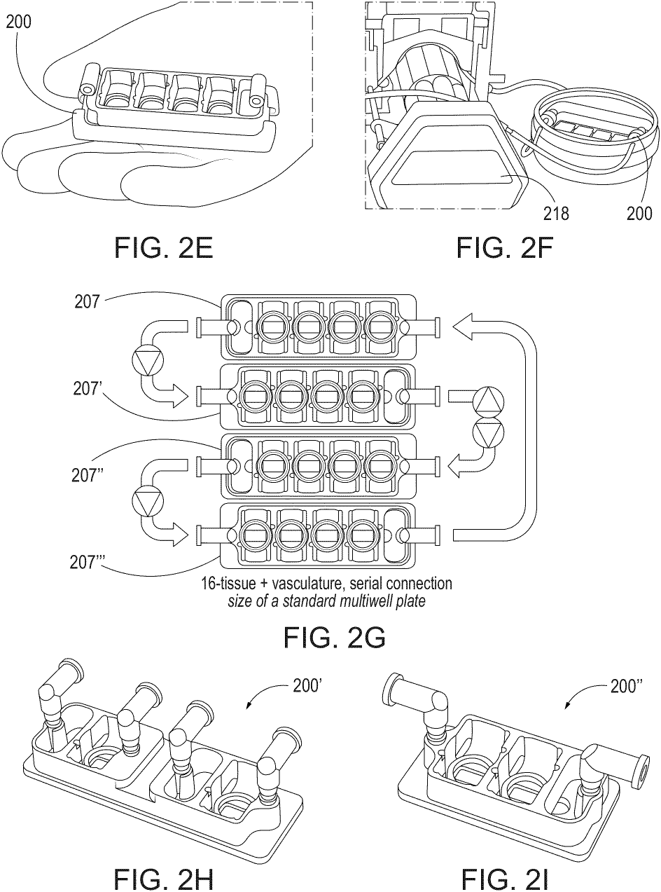

In another embodiment, a configurable, modular kit 200 as shown in FIGS. 2 is provided. The kit 200 comprises a platform 207 having tissue culture chambers, e.g., four tissue culture chambers 208a, 208b, 208c, and 208d, which are suitably shaped to receive corresponding insertable devices 209a, 209b, 209c, and 209d. The platform 207 is configured to engage housing 215 to form kit body. The kit further includes tubular microfluidic connectors 210a and 210b. Tubular microfluidic connectors matingly engage ports 212a and 212b disposed on platform 207 to form a flow path for the vascular media. Tubular microfluidic connectors 210a and 210b when engaged to ports 212a and 212b allow entrance and exit of vascular media through the kit 210. In one embodiment, the tubular microfluidic connectors 210a and 210b have first and second legs forming a substantially right angle. However other angles are suitable such as straight (0 degrees) to fully bent (90 degrees).

FIG. 2B shows a top view of the platform 207 including the tissue culture chambers 208a, 208b, 208c, and 208d. The platform 207 further includes media reservoir 203. Referring to FIG. 2D, in some embodiments, multiple platforms can be integrated into one system to form the footprint of a conventional well plate, such as a 16-tissue platform sized to conform to a standard multiwall plate. In this regard, as shown in FIG. 2G, four platforms 207, 207′, 207″, 207″ can be connected in series. As shown in FIG. 2C, each platform incudes channel 211. The triangle represents a pump, the direction of which is consistent with the arrows they are superimposed onto. From top to bottom, 2G shows a serial flow from the outlet port of one chip to the inlet port of the next. As shown, the platforms are alternated in orientation (inlet on right, inlet on left, inlet on right, inlet on left).

FIG. 2E shows a photograph of the platform 207 having four tissue culture chambers 208a, 208b, 208c, and 208d. and inserted devices 209a-209d disposed in the chambers. The platform is sized to fit in the palm of a hand and thus is both modular and portable. In one embodiment, the bioreactor system, as shown in FIG. 2F, has tubing 217 attached to a pulsatile pump 218 during culture. Other embodiments include a system for a single-organ culture with perfusion and vascular barrier, as shown in FIG. 2H and a dual-tissue culture with perfusion and vascular barrier as shown in FIG. 2I.

In some embodiments, the system is designed for real-time readouts, and can be manufactured of biocompatible, inert materials. The individual culture chambers could be connected in any desired order to model different contexts (FIGS. 1A,B), by using the same outside geometry and customized interiors, leak-free connections and ports.

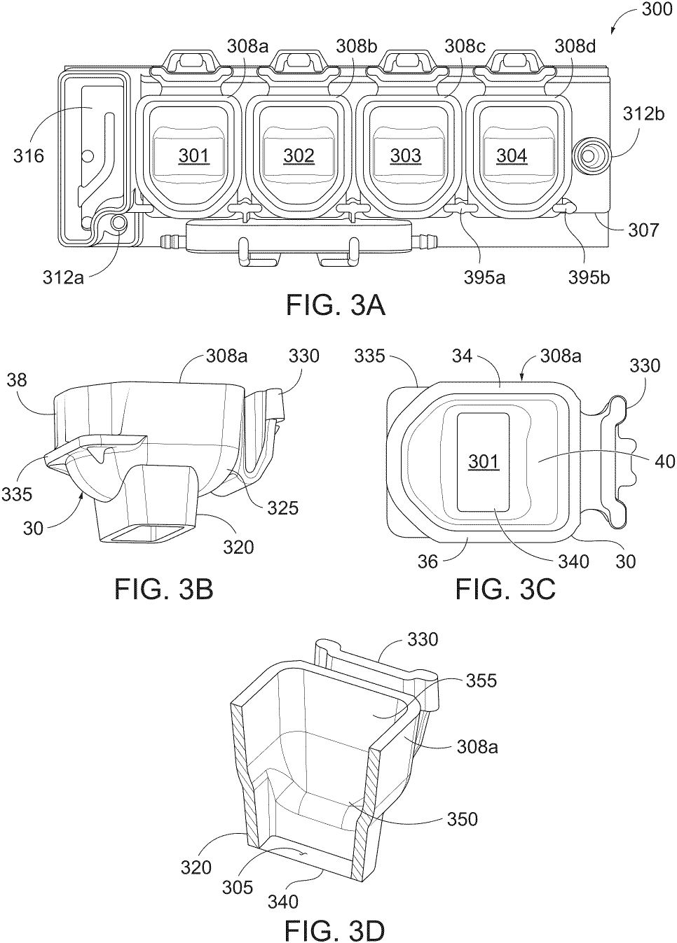

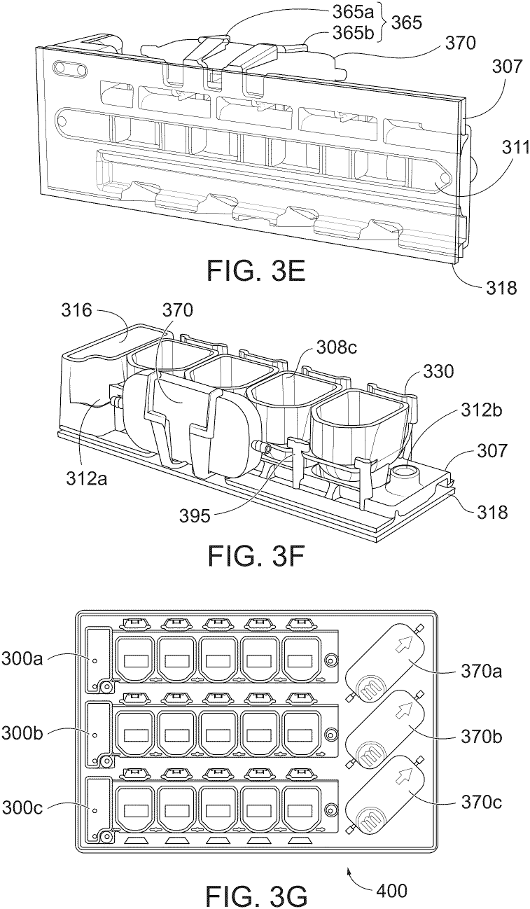

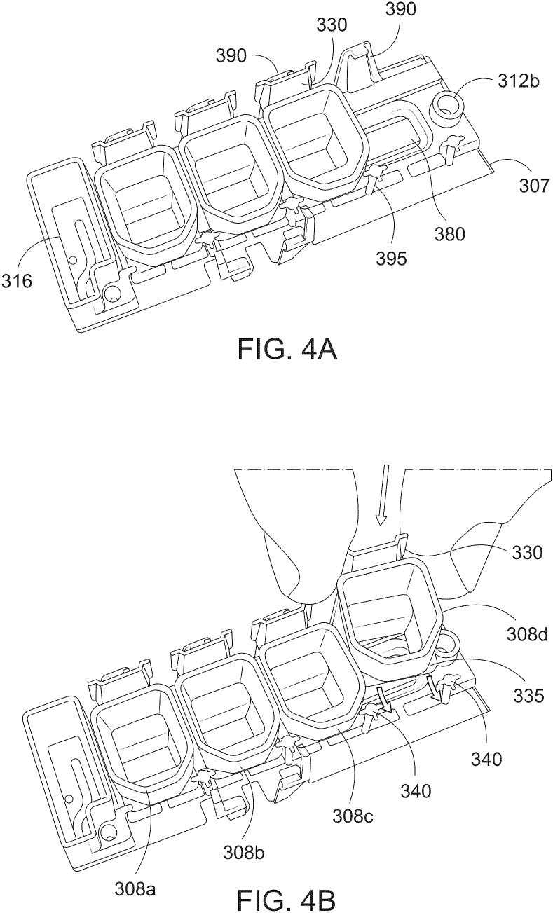

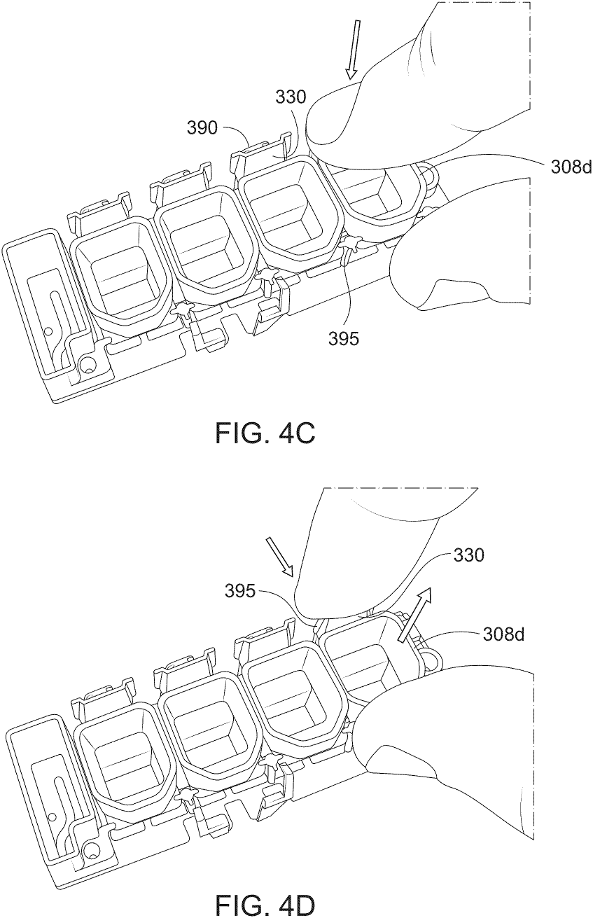

In another embodiment, referring to FIGS. 3A-3G, the system 300 includes modular tissue culture chambers 308a-308d. System 300 includes platform 307 and a plurality of tissue culture chambers 308a-308b. The platform also includes reservoir 316 and ports 312a and 312b. Each tissue culture chamber defines a tissue specific niche, e.g., 301, 302, 303, 304. Tissue culture chambers 308a-308d are configured to releasably mount on platform 307 and platform 307 is configured to releasably receive tissue culture chambers 308a -308b. In this regard, tissue culture chamber 308a, for example, includes a pivoting lever 330 and platform base 307 includes latch 390, as best seen in FIG. 4A. Referring to FIGS. 4B and 4C, an operator can mount tissue culture chamber 308d by installing chamber 308d on the platform 307 by snapping the chamber device into seat 380 (best shown in FIG. 4A) such that latch 390 engages the pivot lever 330 of tissue chamber 308d and ledge 335 engages one or more support members 340 disposed on platform 307.. To remove the chamber 308d from the platform 307, pivotable lever 330 can be pushed or pinched inwardly toward the chamber body to disengage the chamber 308d from platform latch 390 and release the chamber from the platform 307, as shown in FIG. 4D. The removable tissue culture chambers enable transfer of an entire tissue-specific niche by unplugging the chamber from the base. These tissue culture chambers 308a-308d are held side-by-side by seats 380, e.g., receptacles, formed in platform 307.

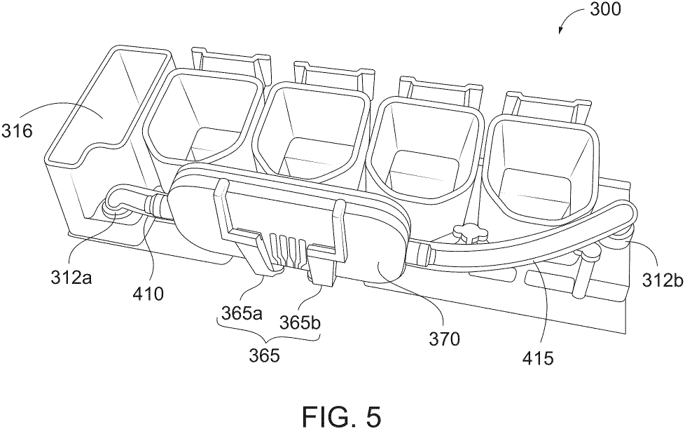

In some embodiments, system 300 may include an onboard pump 400 as shown in FIGS. 3A, 3F and 5. The onboard pump, for example, but not limitation may be a diaphragm. When system includes an onboard pump it enables the entire system to be easily portable as the sytem is an integral system without the need for long wires or connections to a separate pump exterior to the system. Referring to FIG. 3G, the bioreactor system may include multiple systems in series on a single plate. As shown, three platforms 300a, 300b, 300c, are operatively engaged in series, each having its own reservoir for media and pump 370a, 370b, and 370c.

Referring to FIG. 5, onboard pump 400 is disposed on platform 307. In some embodiments, the onboard pump is disposed between one or more tissue culture chambers and a back stand 365 disposed along the platform 307. Back stand 365 may comprise first and second arms 365a and 365b perpendicularly extending upward from platform 307. As shown in FIG. 5, the onboard pump 400 is securely attached to platform 307. The onboard pump 400 is operatively connected to tubing 410 and 415. First tubing 410 is operative connected to onboard pump 400 and entrance port 312a of reservoir 316. Second tubing 415 is operatively connected to pump 400 and exit port 415. In some embodiments, the removable chamber is the insertable device. In this embodiment, the steeper sidewalls so that the entire medium volumentcan be remoed with the insert.

Referring back to FIG. 3B, releasable tissue culture chamber 308a (and 308b-308d) is sized to receive millimeter-sized engineered tissues. Chamber 308 has an integral shell body 30 comprising a first sidewall 34, a second sidewall 36, a front end wall 38, a back end wall 40, and bottom wall 32 that defines a compartment 301 therein. Front end wall 38 comprises ledge 335 extending laterally therefrom and back end wall includes an outer surface comprising pivotable lever 330. The bottom wall includes seat 350 to receive engineered tissue (not shown) and an opening 340. A vascular barrier 305 covers opening 350. In operation, barrier permits cross-talk between tissues in tissue culture chambers 308a-308d when mounted on platform 307, but also permits each chamber to have a tissue-specific niche by inhibiting media from entering the niche or microenvironment defined within the chamber. In some embodiments, chamber 308 includes foot member 320 extending downwardly from the bottom wall of chamber 308. Foot member 320 rests in seat 380 of platform 307 when chamber 308 is mounted into platform 307. In this regard, foot member defines the bottom portion of chamber 308 and includes opening 340 and vascular barrier 305 as shown in FIG. 3D.

In some embodiments, the vascular barrier 105, 205, 305 comprises a mesh insert coated with endothelial cells and supporting mesenchymal stromal cells (MSCs) serving as pericytes. The tissue specific niche defined by the tissue culture chambers vascular is designed to enable crosstalk of drugs, nutrients, secreted factors and cells via pulsatile circulatory flow via pump (e.g., 218 400). Referring back to FIGS. 1C and 1D, the vascular barrier was exposed to increasing levels of hemodynamic shear stress, to establish tight barrier function. Circulating immune cells (human CD14⁺ monocytes) were able to extravasate from the vascular flow and target sites of tissue injury (FIG. 1E).

Tissue maturity, a necessary condition for recapitulating physiological tissue responses in an adult human, has been notoriously difficult to achieve using iPSC-derived cells. Therefore, multi-cellular, millimeter-sized human tissues were formed and matured over 4-6 weeks of culture before being transferred into the system, allowing different maturation regimens and culture durations and a quality control step before integration.

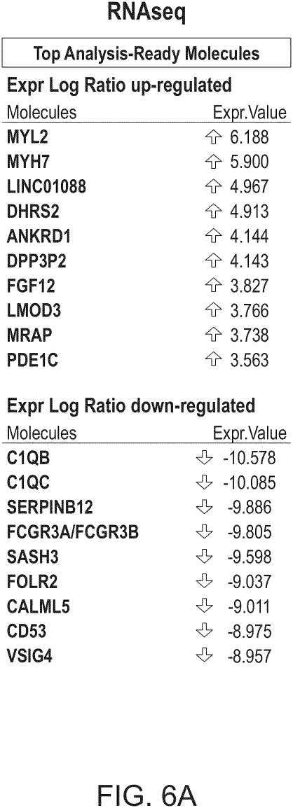

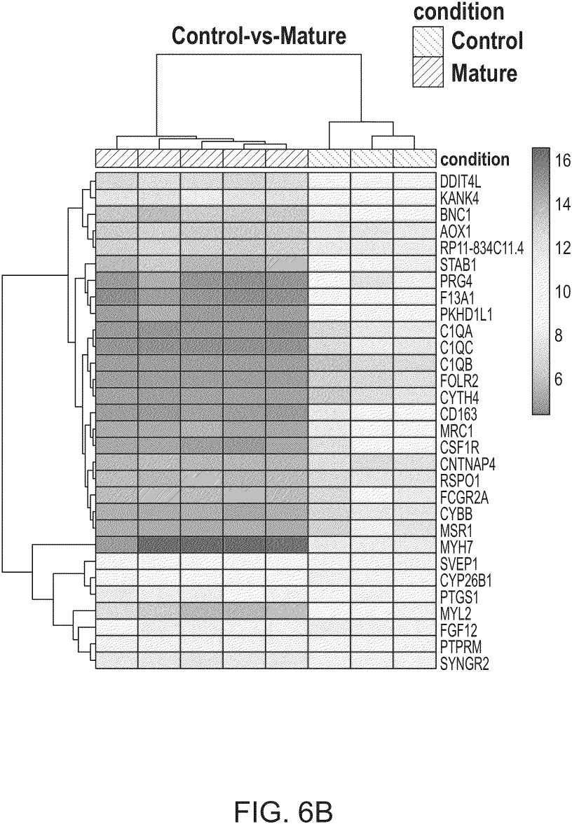

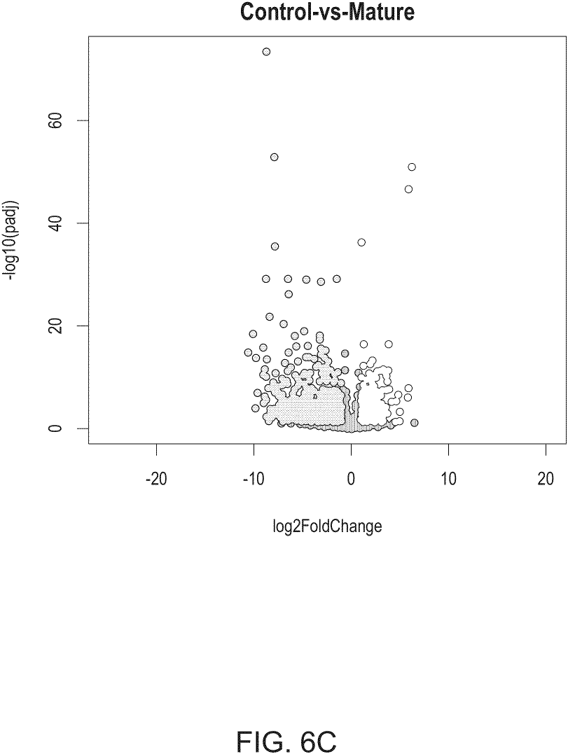

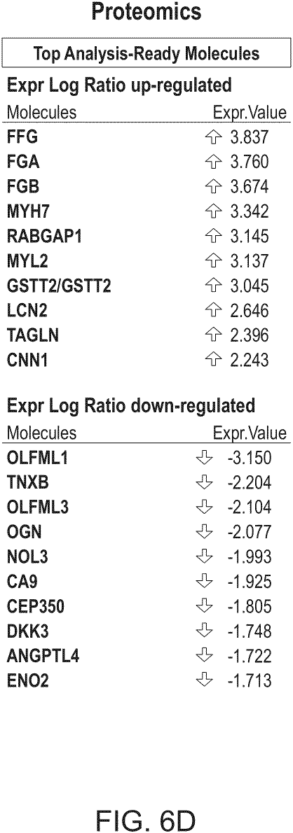

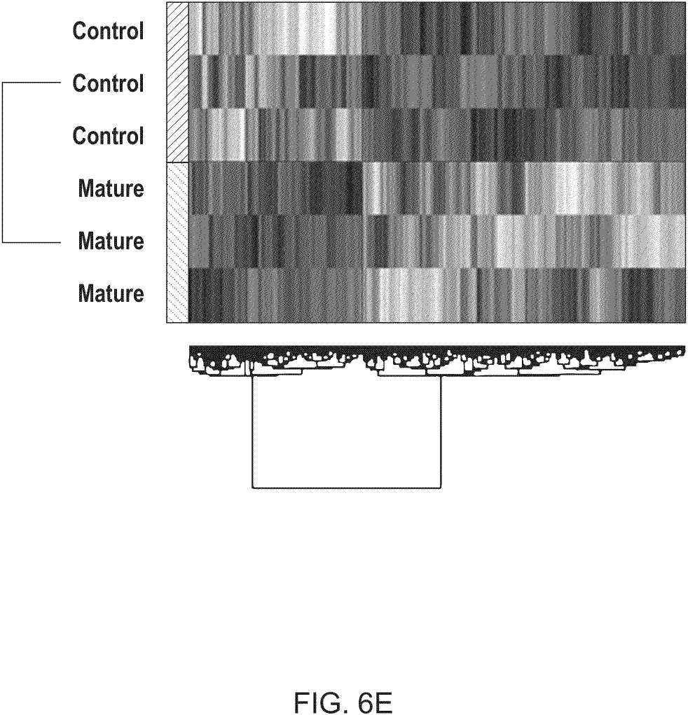

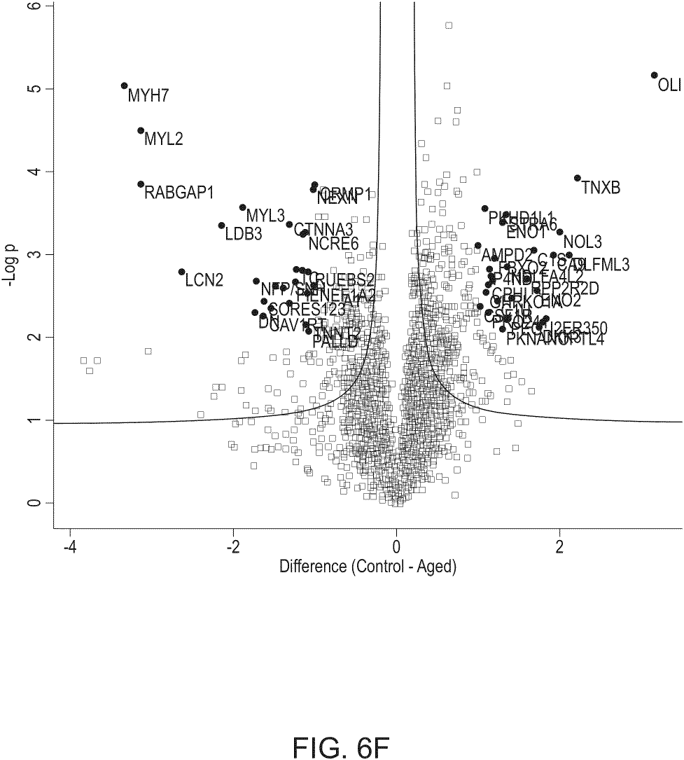

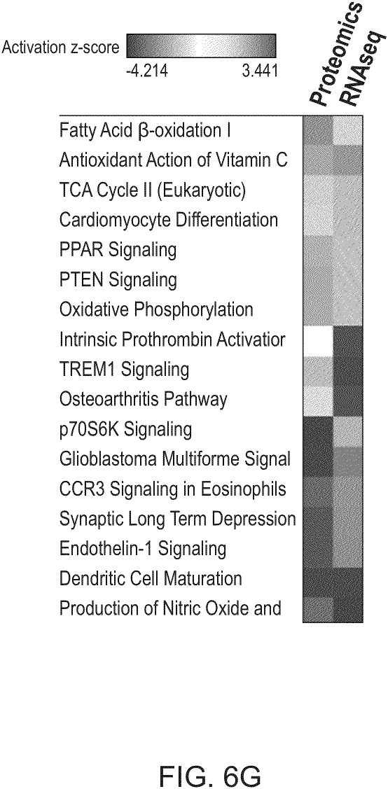

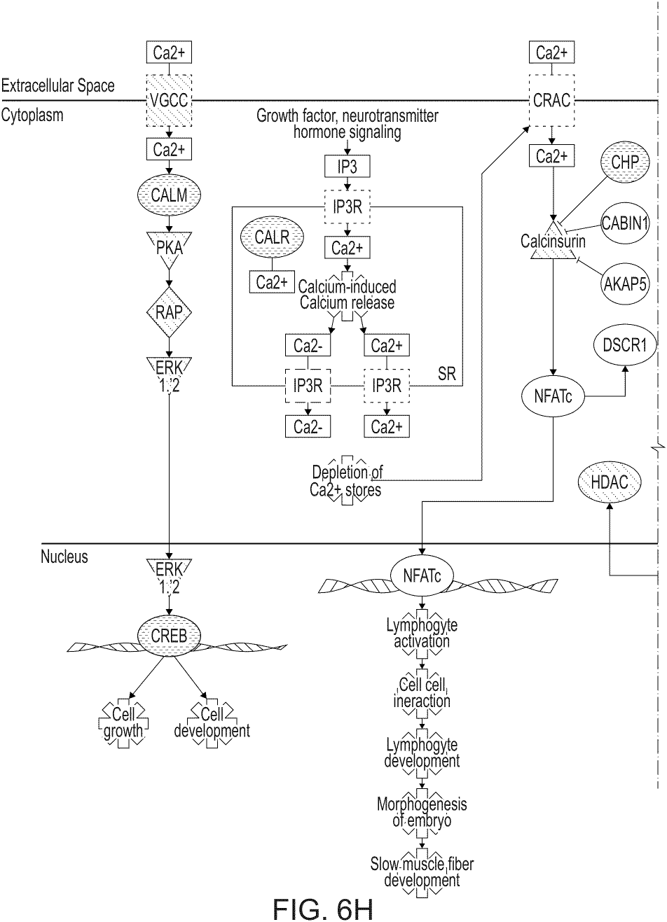

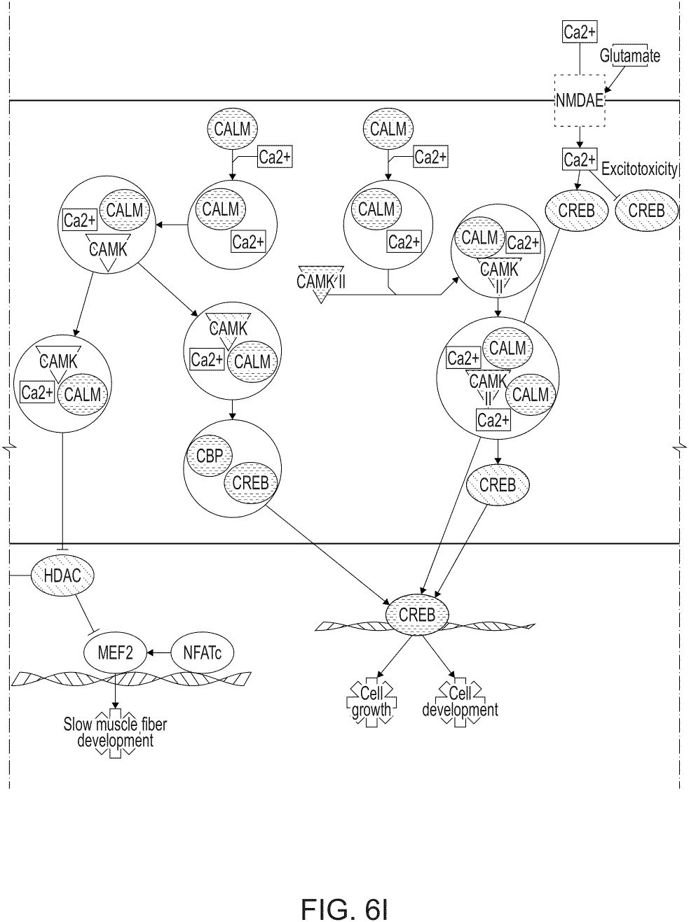

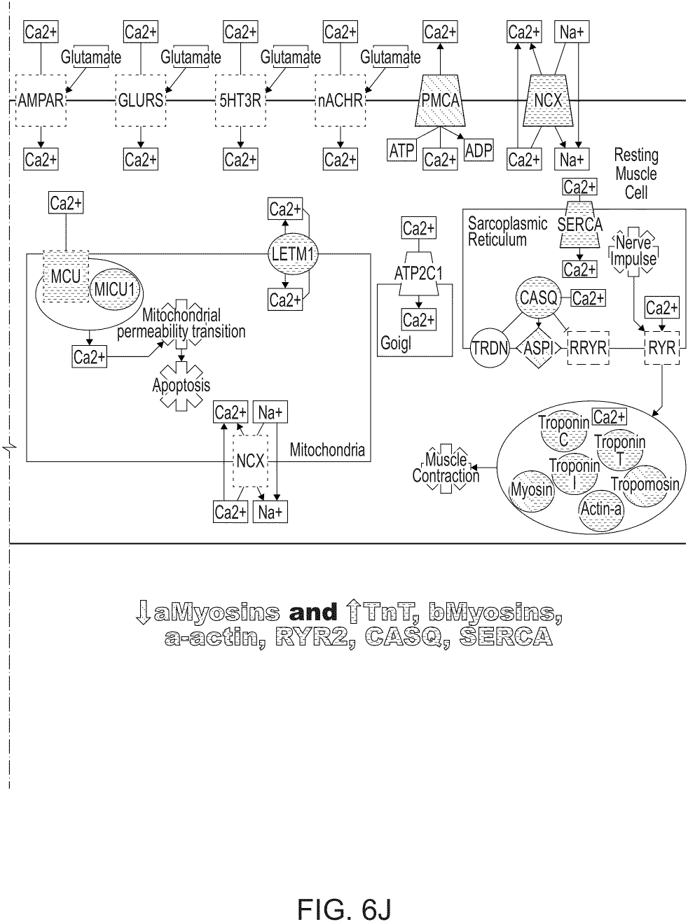

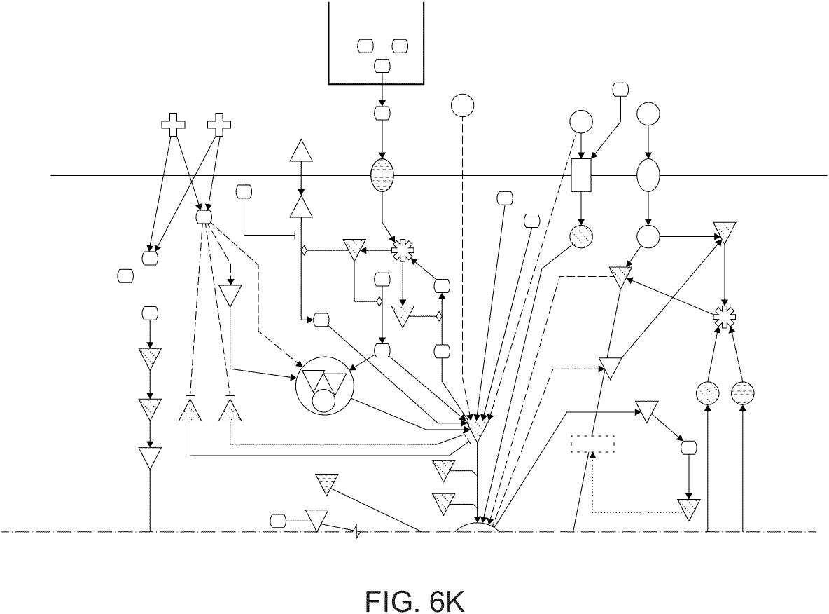

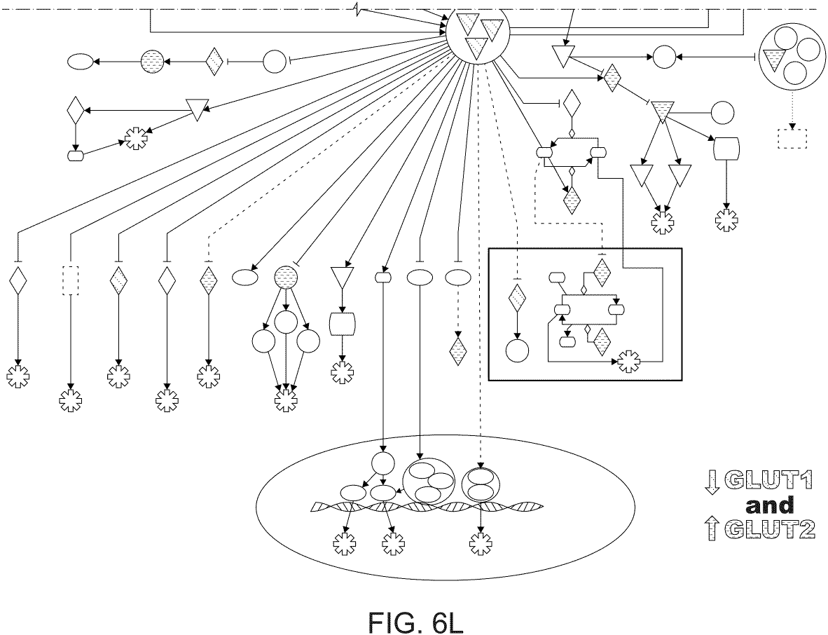

For example, adult-like human iPSC-derived cardiac muscle displayed mature ultrastructure, metabolism and calcium signaling compared to non-matured controls. FIGS. 6A-L show cardiac maturation as a result of electromechanical stimulation at an increasing intensity. FIGS. 6A-C show RNA sequencing and FIGS. 6D-F show proteomics of engineered cardiac tissues demonstrates the effects of maturation on cardiac phenotype. FIG. 6G shows that proteomic and RNAseq datasets show comparable pathway regulation with matured tissues, based on upregulated (FIGS. 6H-J) calcium signaling and ultrastructure and (FIGS. 6K-L) more adult-like metabolic activity.

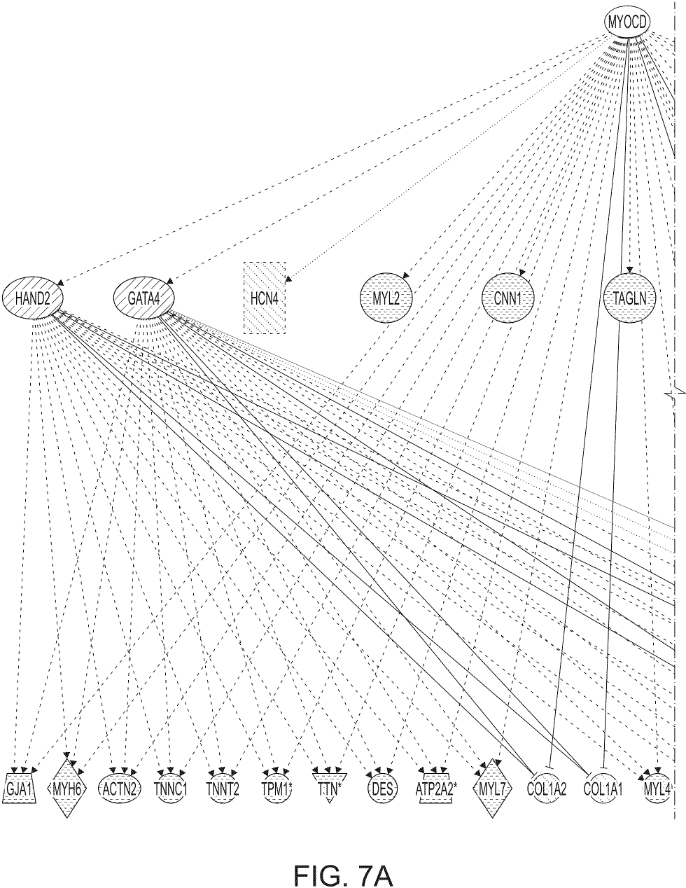

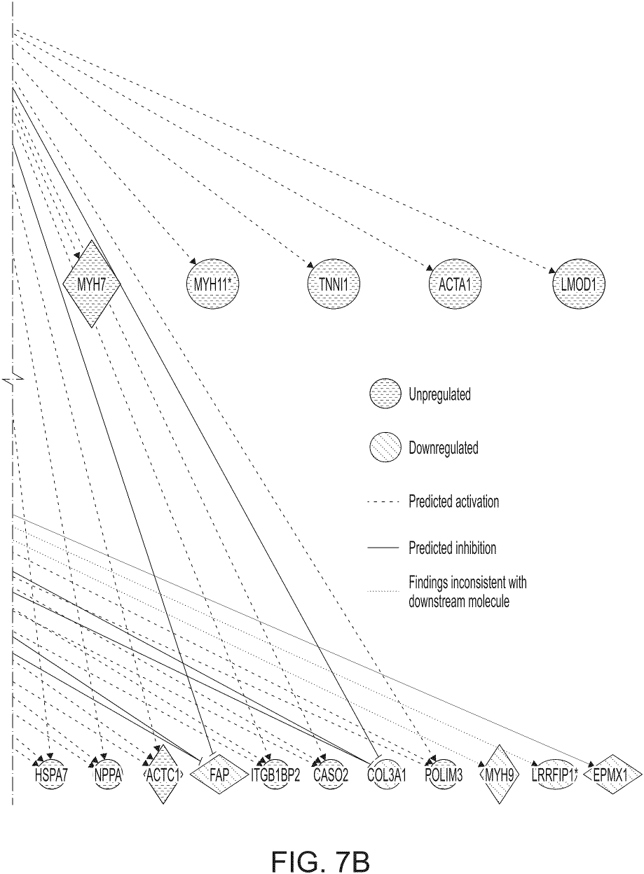

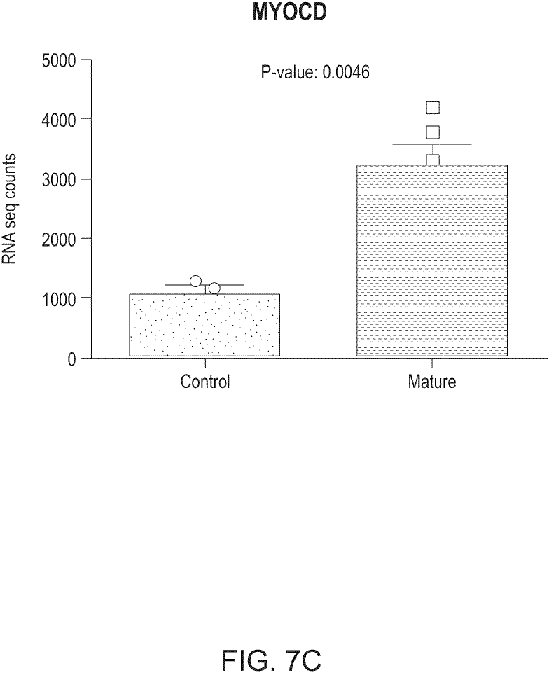

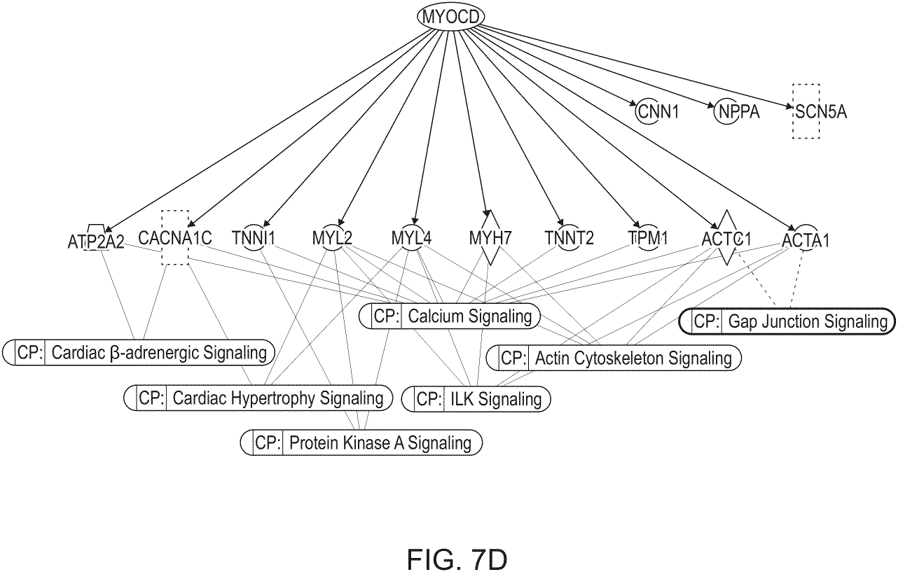

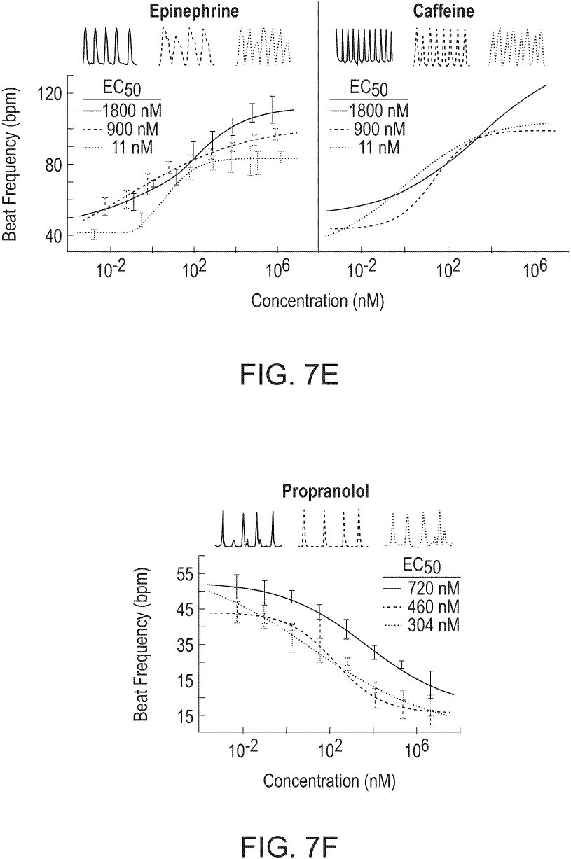

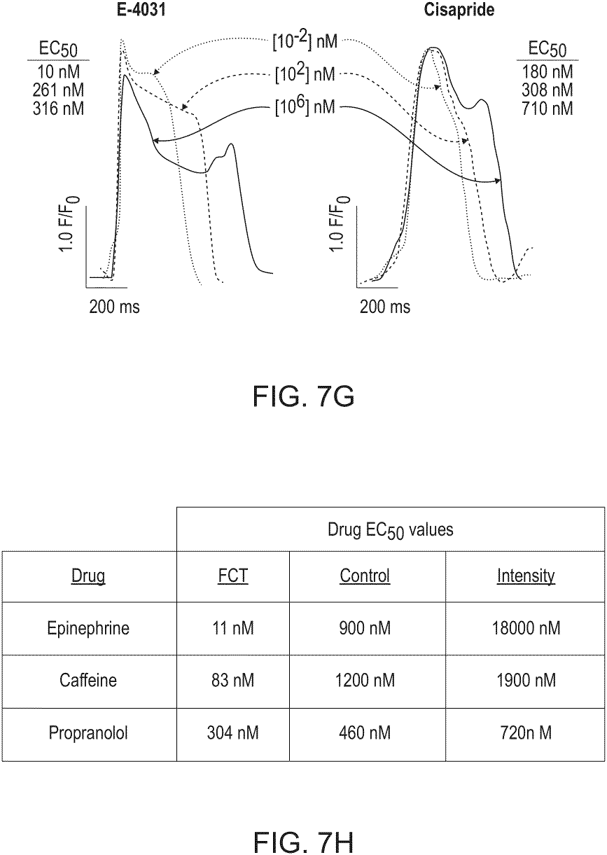

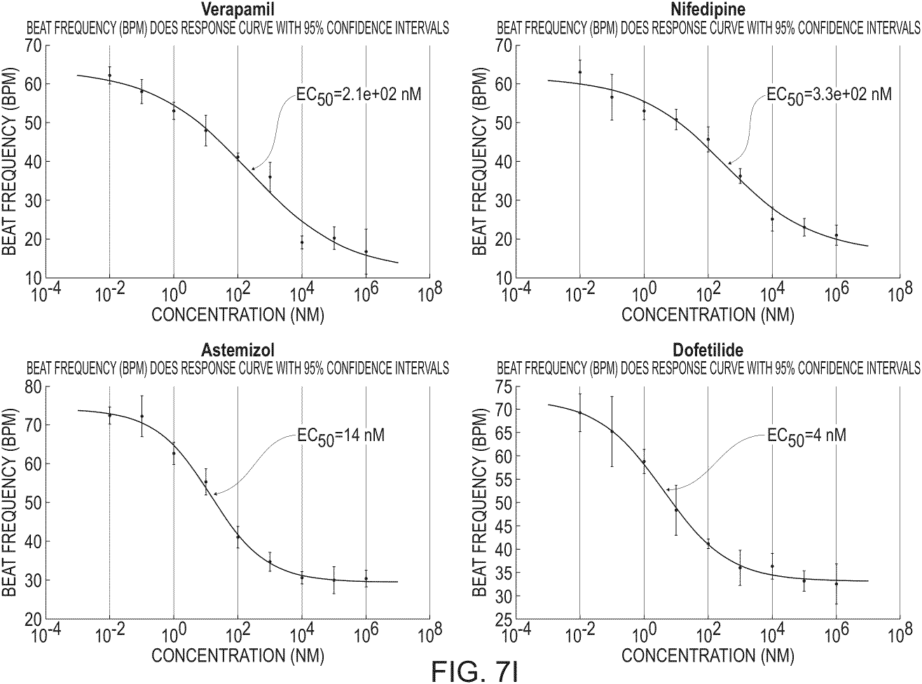

FIGS. 7A-I show that myocardin regulates cardiac maturity to enable recapitulation of clinically observed drug responses. FIGS. 7A-B show that Ingenuity pathway analysis reveals myocardin (MYOCD) as an upstream regulator, activating HAND2 and GATA4, to upregulate many key genes involved in mature cardiac functionality. FIG. 7C shows that myocardin is upregulated in matured cardiac tissues and FIG. 7D shows that myoxardin is associated with activation of genes related to proper drug responses. Drug responses were validated experimentally by comparing matured engineered cardiac tissues (Blue), control engineered cardiac tissues (red), and human fetal cardiac tissues (FCT) aged 17 weeks (green) for (FIG. 7E) cardiac ionotropic drugs, (FIG. 7F) beta blockers, (FIG. 7G) QT prolonging drugs. FIG. 7H shows a table displaying the EC50 values for these drugs. FIG. 7I shows that mature tissues were further validated by measuring dose-dependent responses (shown as mean with 95% confidence interval) to calcium signaling drugs, using calcium channel blockers.

Maturation appeared to be regulated by myocardin, GATA4 and HAND2 that activated genes related to adult-like cardiac function (FIGS. 7A-D). We found that matured cardiac tissues recapitulated clinical EC50 values (FIGS. 7E-H). For example, these tissues recapitulated bradycardiac effects of calcium channel blockers seen in patients (FIG. 7I), in contrast to cell monolayers that showed tachycardic effects.

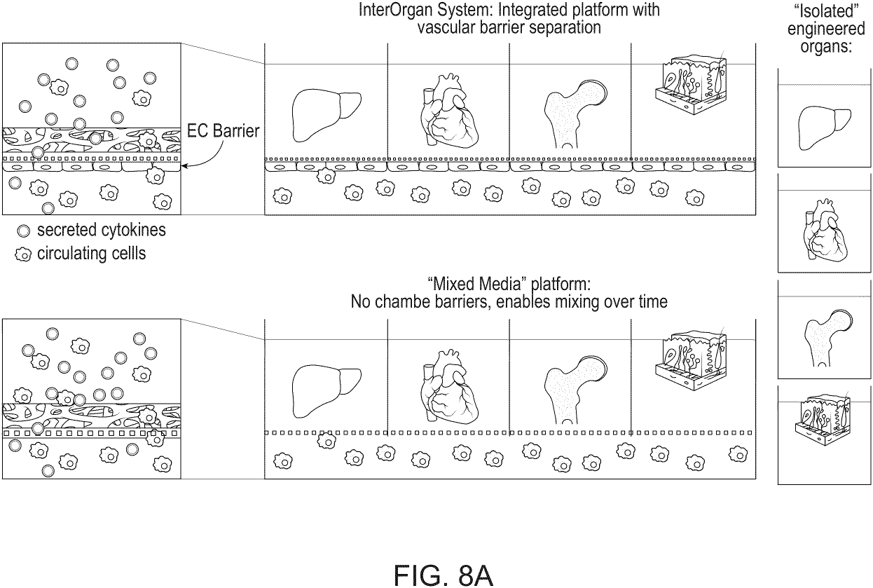

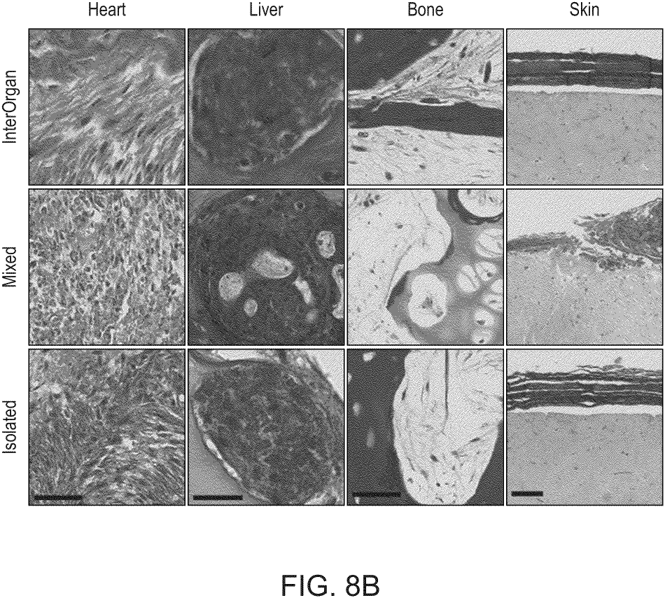

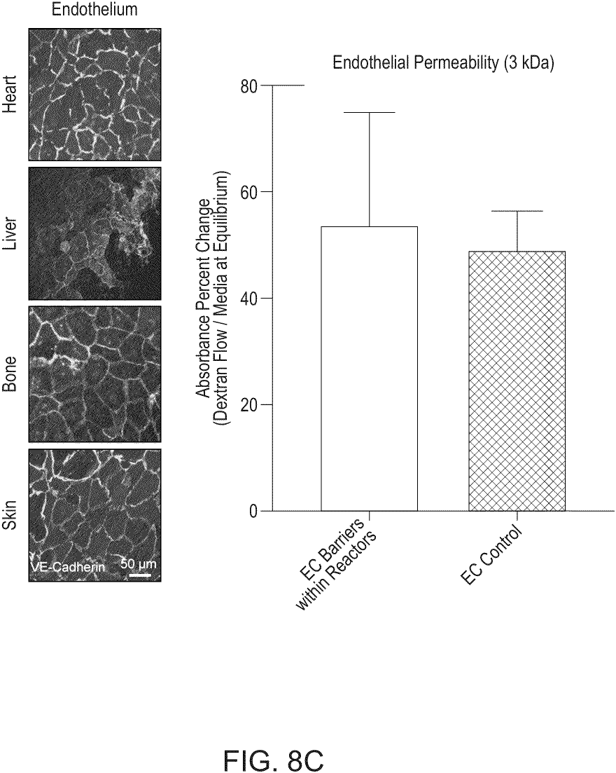

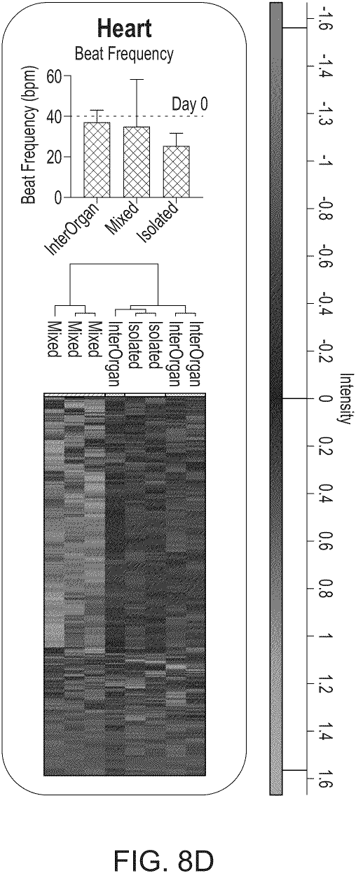

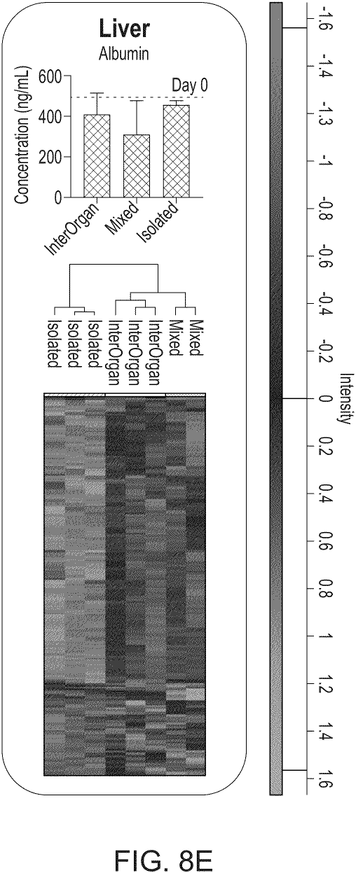

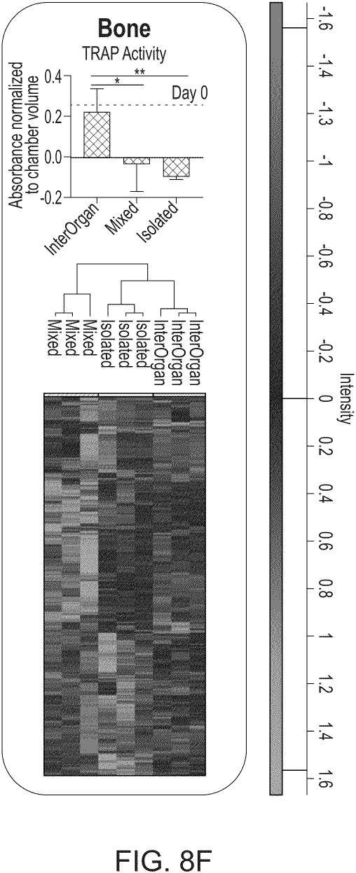

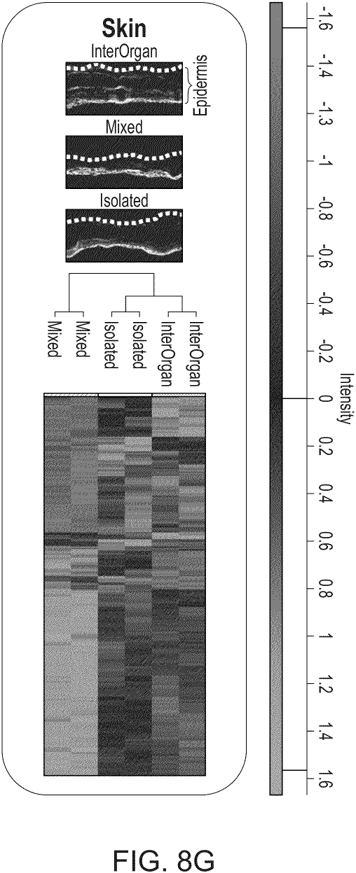

FIGS. 8A-G show maintenance of structural, functional, and molecular phenotypes of engineered tissues over 4-week culture. FIG. 8A shows morphological differences between groups (Trichrome; scale = 50 µm for heart, liver, bone; 100 µm for skin). FIGS. 8A-G show that vascular phenotypic gap junctions and barrier stability were maintained after 4-week culture (VE-Cadherin; scale = 50 µm). (8D-G) Functional and molecular features for each engineered tissue and culture condition (n=5). * p <0.05; ** p <0.01.

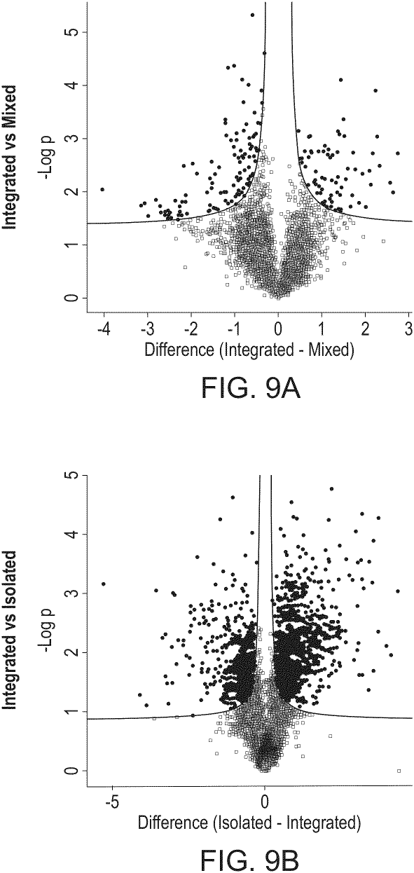

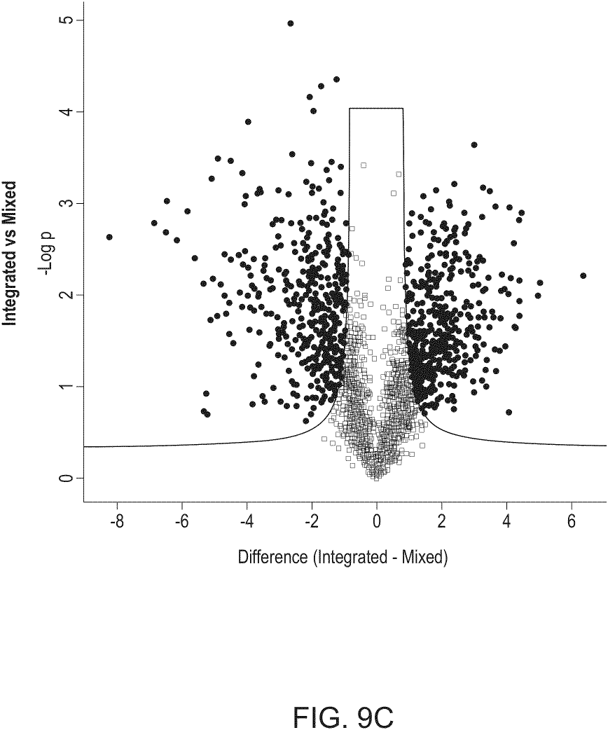

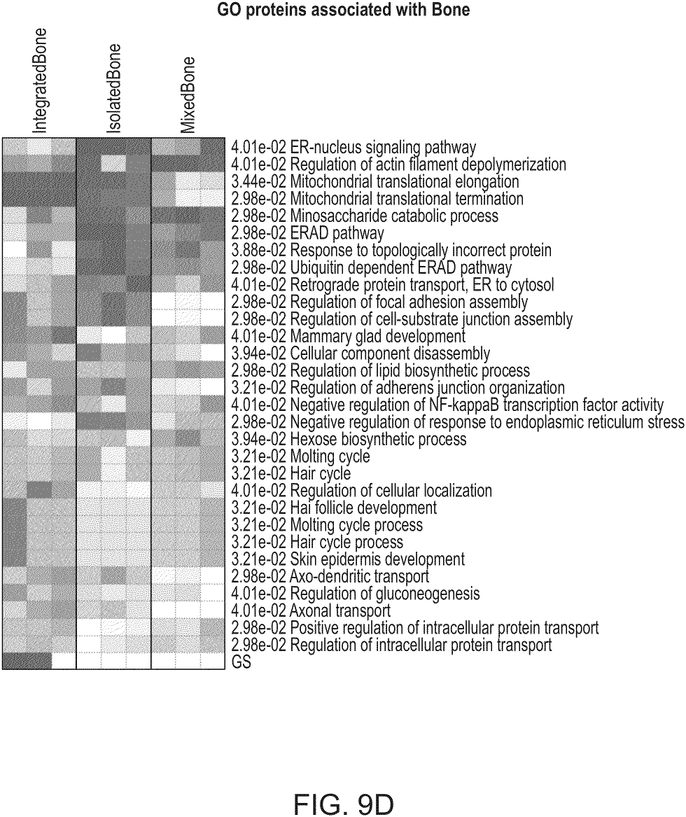

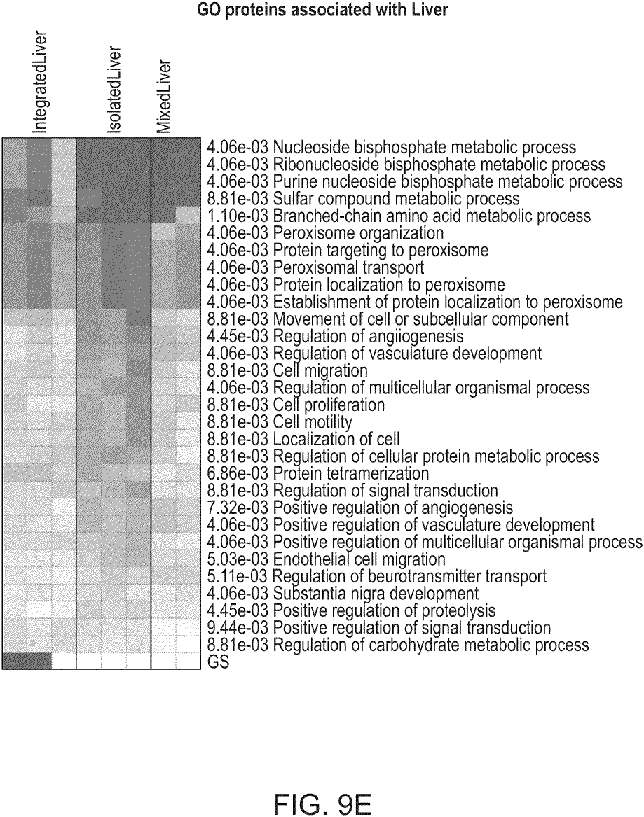

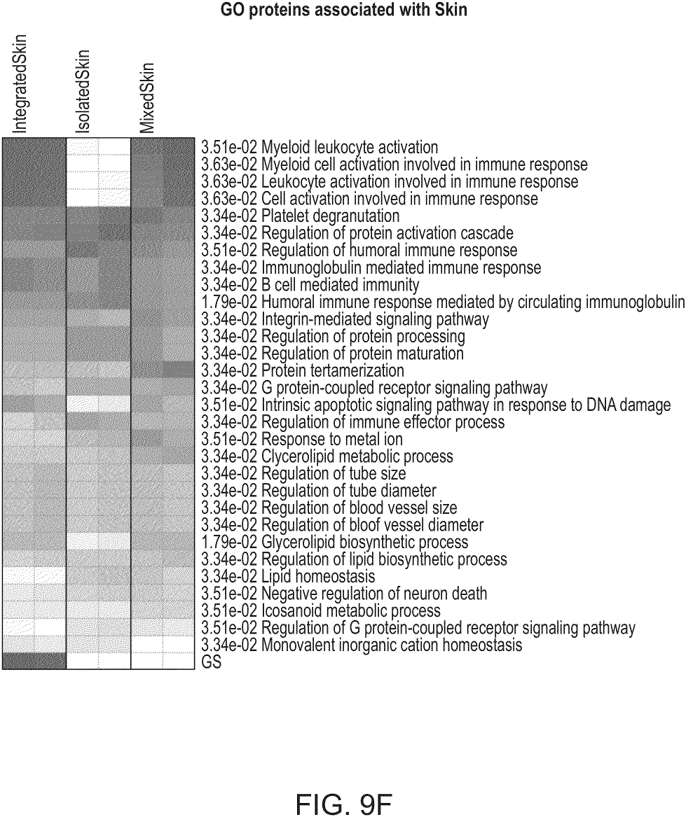

FIGS. 9A-F show staining of InterOrgan, Mixed, and Isolated tissue samples after 28 days of culture, as described in FIGS. 8. Staining of heart (alpha-actinin = green; scale = 10 um), liver (albumin = green; CYP450 = red; scale = 50 um), bone (picrosirius red; scale = 50 um), and skin (keratin 14 = red; vimentin = green; scale = 50 um) tissues.

The system described herein containing tissues connected by vascular perfusion and endothelial barrier between tissues and perfusate (InterOrgan) was systematically compared to the identical system without endothelial barriers (Mixed conditions corresponding to the co-culture in common medium), and tissues cultured in isolation, without perfusion (Isolated condition, benchmark for phenotype stability) over 4 weeks. In the InterOrgan platform, the selectively permeable endothelial barriers enabled tissue communication while preserving tissue-specific biological fidelity. In contrast, the Mixed condition normalized the medium composition throughout all compartments (Table 1 below). This condition failed to preserve tissue-specific structural, functional, and molecular markers, demonstrating the need for maintaining tissues in their individual niches. Thus, the system embodied and claimed herein exhibited unexpected superior results.

TABLE 1

Culture media compositions. The platform contained 1.5 mL of media in each tissue compartment (liver, heart, bone, skin) and 12 mL of circulating media

| | | |
|---|---|---|
| CIRCULATING ENDOTHELIAL MEDIA: | CARDIAC TISSUE MEDIA: | BONE TISSUE MEDIA: |
| Endothelial Basal Media (EBM) | RPMI | EMEM Basal Media |
| EGM-2 Media Supplement: | Pennicillin/Streptomycin | Fetal Bovine Serum |
| Fetal Bovine Serun | B27 Supplement: | Pennicillin/Streptomycin |
| Hydrocortisone | Biotin | MCSF |
| hFGF-B | DL Alpha Tocopherol Acetate | sRANKL |
| VEGF | DL Alpha-Tocopherol | |
| R3-IGF-1 | Vitamin A (acetate) | |
| Ascorbic Acid | BSA, fatty acid free Faction V | SKIN TISSUE MEDIA: |
| hEGF | Catalase | DMEM/F12 |
| GA-1000 | Human Recombitant Insulin | Adenine |
| Heparin | Human Transferin | Hydrocortisone |
| | Superoxide Dismutase | T3 (triodo-I-thyronine) |
| | Corticosterone | Insulin-Transferin-Selenium (ITS) |
| LIVER TISSUE MEDIA: | D-Galactose | Ascorbic Acid |
| Hepatic Basal Media (HBM) | Ethanolamine HCL | Ethanolamine and Phosphooryethanolamine (EOP) |
| HCM Media Supplement: | Glutathione (reduced) | Calcium Chloride |
| Transferin | L-Carnitine HCl | Fetal Bovine Serum |
| Ascorbic Acid | Linoleic Acid | Pennicillin/Streptomycin |
| Insullin | Linolenic Acid | |
| Hydrocortisone | Progesterone | |
| BSA (Fatty-Acid Free) | Putrescine 2HCl | |
| GA-1000 | Sodium Selenite | |
| | T3 (triodo-I-thyronine) | |

Overall, each tissue in the InterOrgan bioreactor system group, and markedly exceeded the corresponding properties of the Mixed group (FIGS. 7A-I, FIGS. 8A-G). Specifically, cardiac muscle showed increased cell elongation and striations in the InterOrgan and Isolated groups as compared to the Mixed group (FIGS. 7A, 8A-G). Bone tissues in the InterOrgan and Isolated groups displayed mature osteolytic phenotype, while the Mixed group showed decreased collagen deposition. Similarly, liver tissue morphology was comparable for the InterOrgan and Isolated groups and inferior for the Mixed group. The epidermal layer of the skin remained intact in the InterOrgan and Isolated culture, whereas in the Mixed platform it showed disruptions. The functionality of tissues over 4 weeks of culture were comparable for InterOrgan and Isolated groups: heart tissues maintained beating rate, liver tissues maintained albumin secretion, bone tissues maintained TRAP activity, and skin tissues demonstrated macrophage-stimulating release of MIP-3a and comparable epidermal thickness (FIGS. 1D, 2D-G).

FIGS. 9A-F show proteomic breakdown for three of the four tissues. FIG. 9A shows engineered bone (6,000+ unique proteins), FIG. 9B shows liver (2,000+ proteins), and FIG. 9C shows skin (2,000+ proteins) studied over 28 days in the (i) integrated multi-tissue platform, (ii) platform with mixed media, and (iii) tissues cultured in isolation. Comparison of integrated versus mixed conditions via differential protein abundances is represented by Volcano plots. (FIGS. 9D-F) PGSEA plots of the top 30 GO Biological Process pathways, with red indicating activated pathways and blue indicating suppressed pathways for bone (FIG. 9D), liver (FIG. 9E), and skin (FIG. 9D).

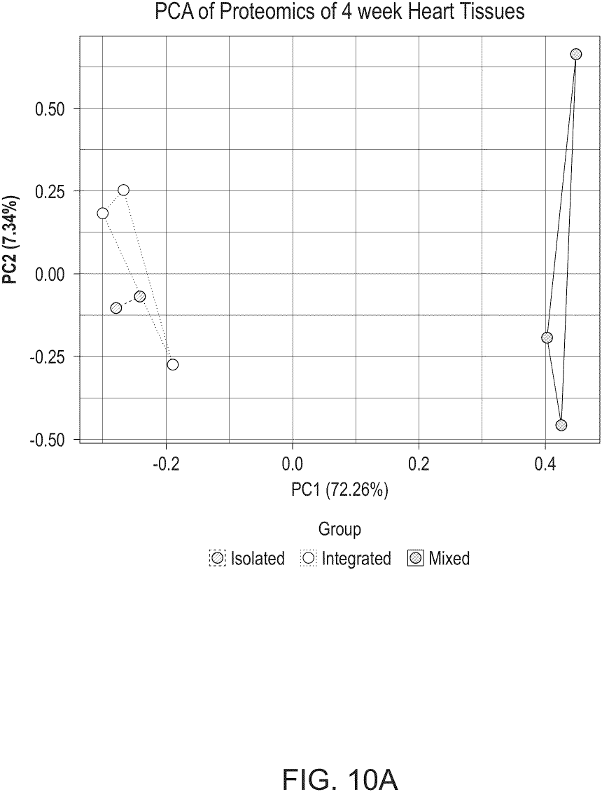

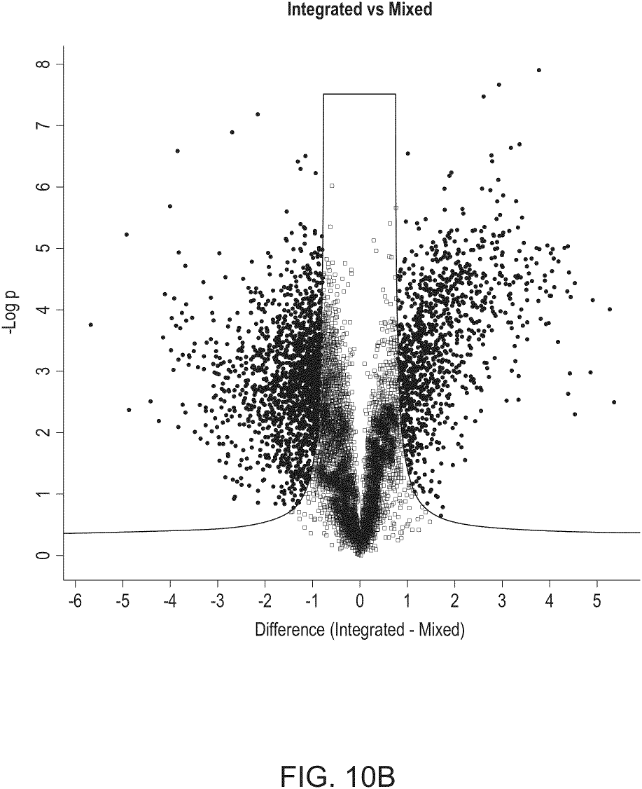

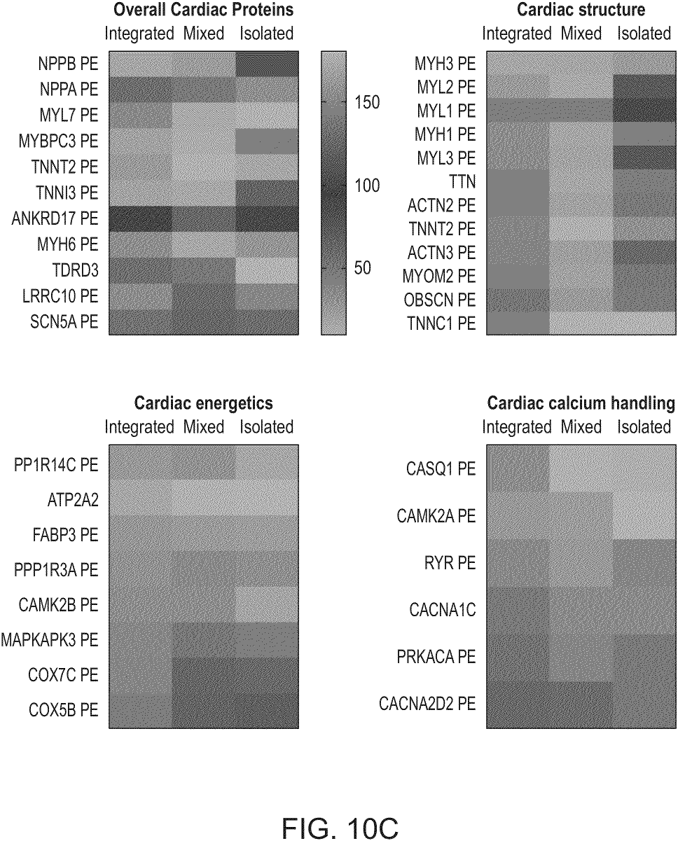

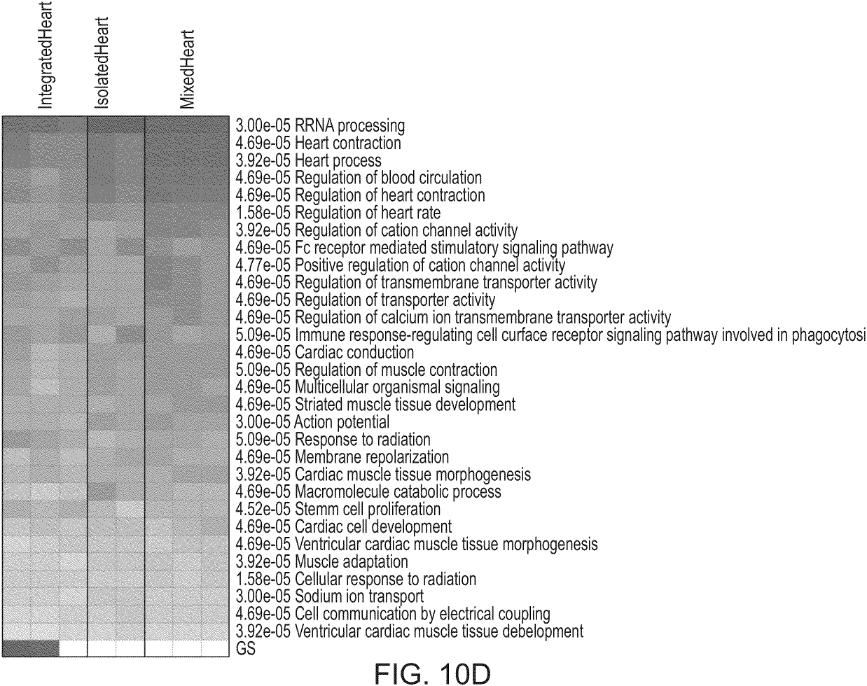

FIGS. 10A-D show proteomic breakdown for cardiac tissue. Data are shown for 28 days of cultivation in (i) integrated multi-tissue platform, (ii) platforms with mixed media, and (iii) tissues cultured in isolation. FIG. 10A shows PCA clustering for each experimental condition (i-iii). FIG. 10B shows a comparison of integrated versus mixed conditions via differential protein abundances. FIG. 10C shows proteins important to cardiac tissue function, structure, energetics, and calcium handling. FIG. 10D shows PGSEA pathway analysis showing the top 30 GO Biological Process pathways related to disease and function in integrated vs. mixed conditions, with red indicating activated and blue indicating suppressed pathways.

By whole proteome analysis, we detected thousands of proteins expressed in each engineered tissue (heart: ~6,000; liver: ~4,000; bone: ~5,000; skin: ~2,000), and differentially expressed between the InterOrgan and Mixed media conditions (FIGS. 7D-G, 9, 10).

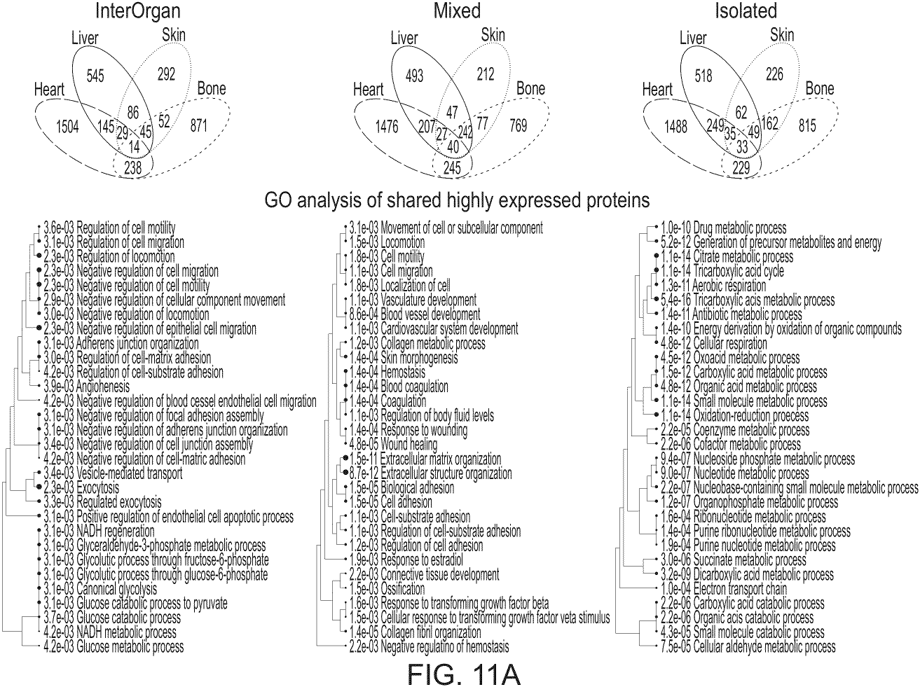

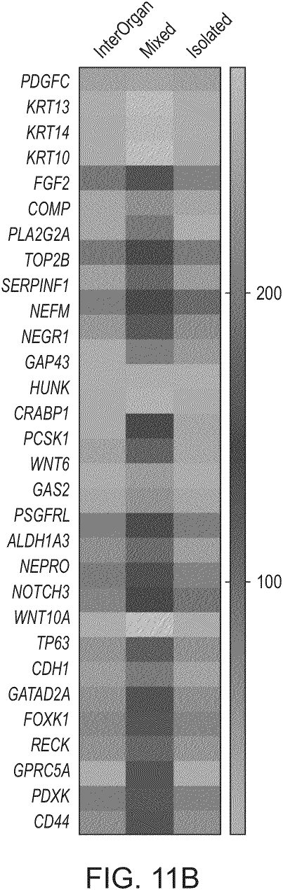

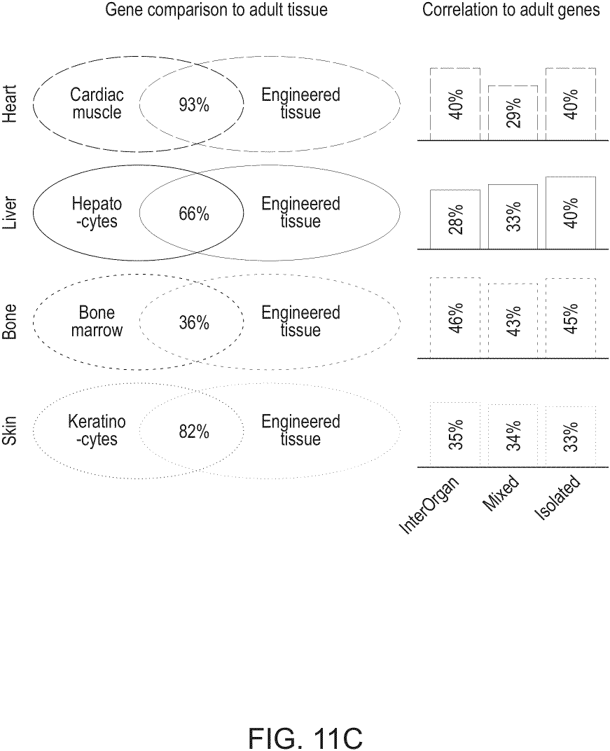

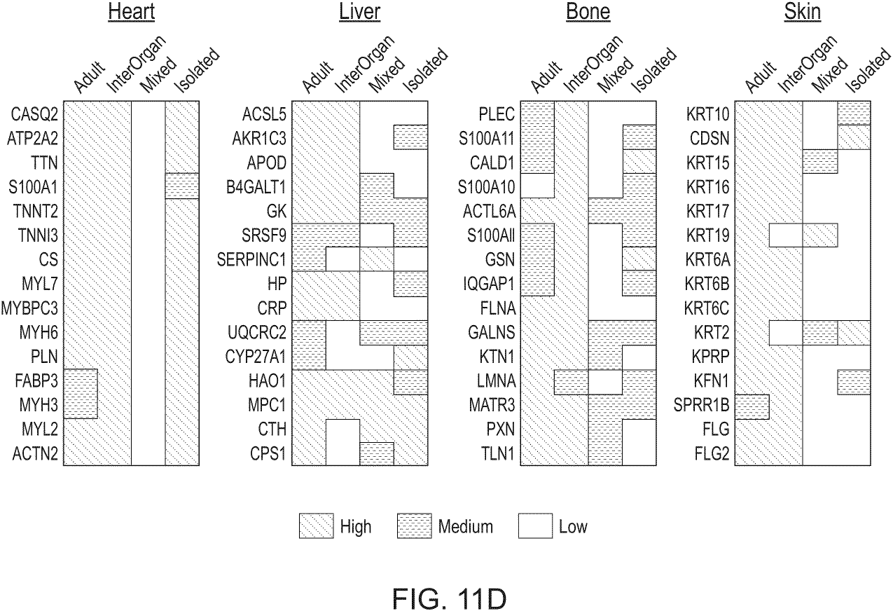

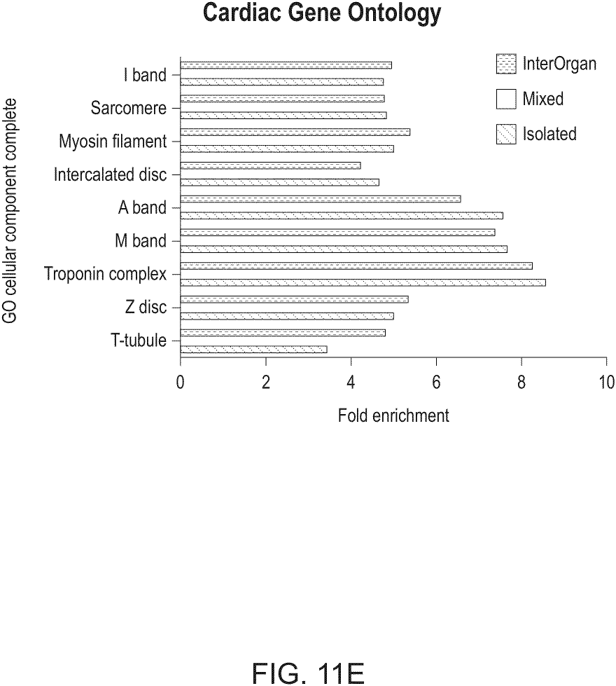

FIGS. 11A-E show that proteomic analysis confirms biological fidelity of InterOrgan platform in comparison to isolated cultures and adult human tissues. FIG. 11A shows that within each group, GO analysis identified gene pathways shared among different tissues. FIG. 11B shows that cardiac tissues in the Mixed group contained proteins that are often found in off-target tissues (osteochondral, neural, etc) FIG. 11C shows that similarities in overlapping genes between published adult data and engineered tissues (left); expression within each experimental group correlates to adult tissue (right). FIG. 11D shows that within each tissue, top proteins of interest were compared to adult tissue. FIG. 11E shows that for cardiac tissues, GO analysis identified cardiac-specific, adult-like structural components only in the InterOrgan and Isolated groups.

Through Ingenuity Pathway Analysis (IPA) and gene ontology (GO), we identified common genes expressed in all tissues among the three conditions (InterOrgan, Mixed, Isolated) (FIG. 11A). The top genes shared among all tissues under the InterOrgan and Isolated conditions were primarily related to normal physiological functions (such as metabolism), while genes expressed under the Mixed condition were related to off-target organ functions (FIG. 11A). In particular, proteins associated with epithelial, neurogenic and osteochondral development were markedly higher in the cardiac tissues under the Mixed conditions, presumably due to the presence of growth factors from adjacent tissues in the absence of an endothelial barrier (FIG. 11B). GO analysis of cellular components revealed that only InterOrgan and Isolated conditions supported the maintenance of mature cardiac phenotype, evidenced by enriched contractile proteins and T-tubules (FIG. 11E), supporting the need to preserve tissue-specific niches.

Tissue-specific proteins expressed in adult and engineered tissues were identified and tertiled using the Human Protein Atlas. All engineered tissues in the InterOrgan platform matched well to the published correlates from adult donors (FIG. 11C). Overall, engineered tissues cultured in the InterOrgan platform better matched the repertoire of highly abundant genes in adult heart, liver, bone and skin (FIG. 11D).

Having established the maintenance of tissue-specific functionality along with tissue cross-talk over 4 weeks of culture, the system described herein provided a high-fidelity in vitro mimic of human physiology for drug testing or disease modeling. To mimic injection, drugs were delivered from the reservoir to the tissues by vascular circulation. The platform incorporated first-pass liver metabolism and on- and off-target drug effects on all tissues.

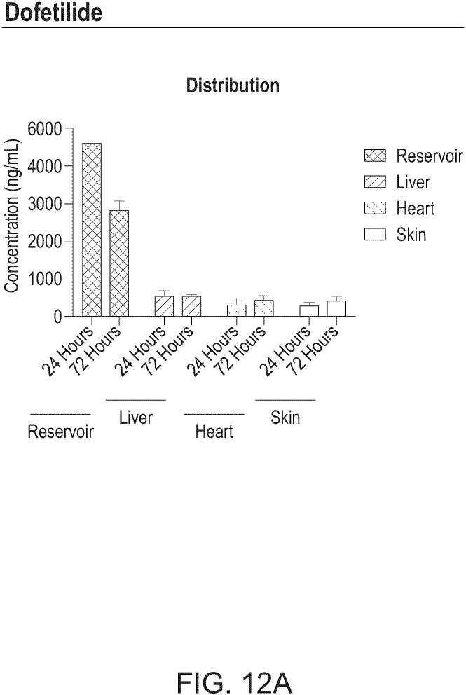

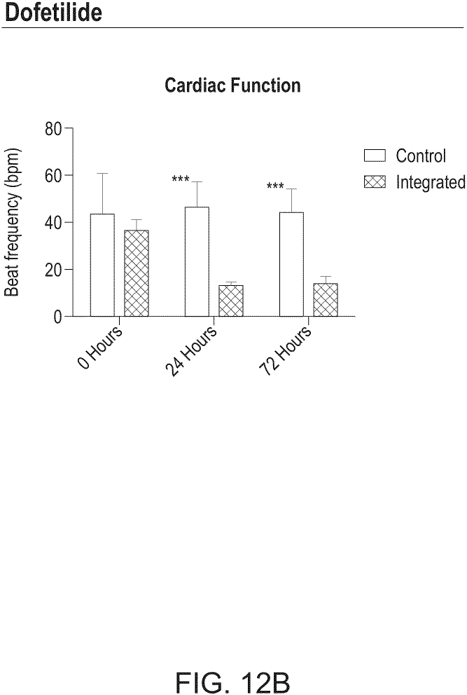

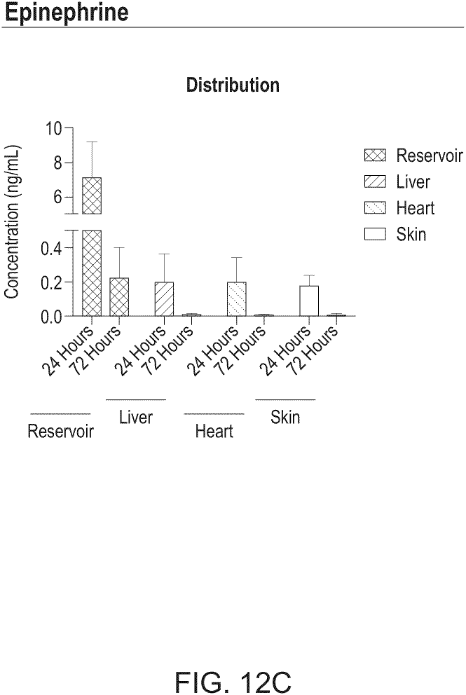

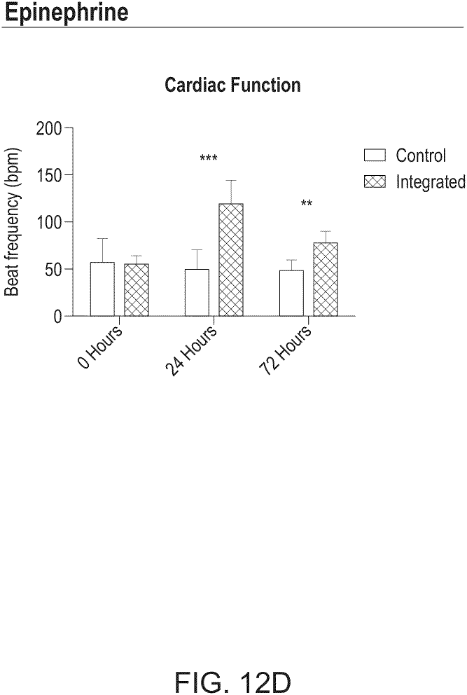

FIGS. 12A-D show drug distribution in the platform measured by LC-MS quantification of drug concentrations with functional matching. Distribution of 10 uM dofetilide injected into the reservoir at t = 0 was studied, and dofetilide drug concentration (FIG. 12A) and cardiac beat frequency (FIG. 12B) were assessed at t = 24 and 72 hours. For epinephrine, a 10 µM dose was introduced into the reservoir at t = 0, and epinephrine drug concentration (FIG. 12C) and cardiac beat frequency (FIG. 12D) were assessed at t = 24 and 72 hours.

Addition of dofetilide, a selective hERG K⁺ channel blocker, decreased beat rate of cardiac muscle and metabolic drug clearance in the liver (FIGS. 12A,B). Similarly, the β-adrenergic agonist epinephrine increased the beat rate in a time-dependent manner (FIGS. 12C,D).

The InterOrgan platform allows screening for both on-target and off-target drug effects. We chose to demonstrate the off-target effects of a common anti-cancer therapeutic doxorubicin (Dox), which shows cardiotoxicity in subsets of patients, limiting its broad clinical use.

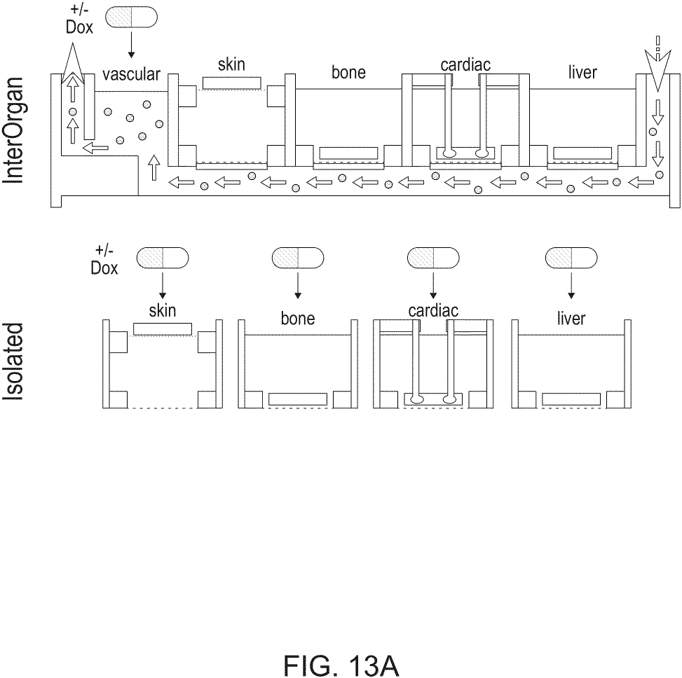

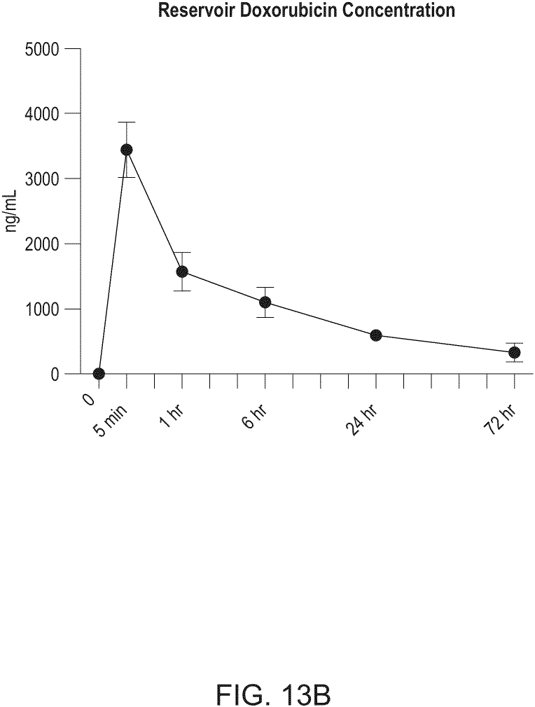

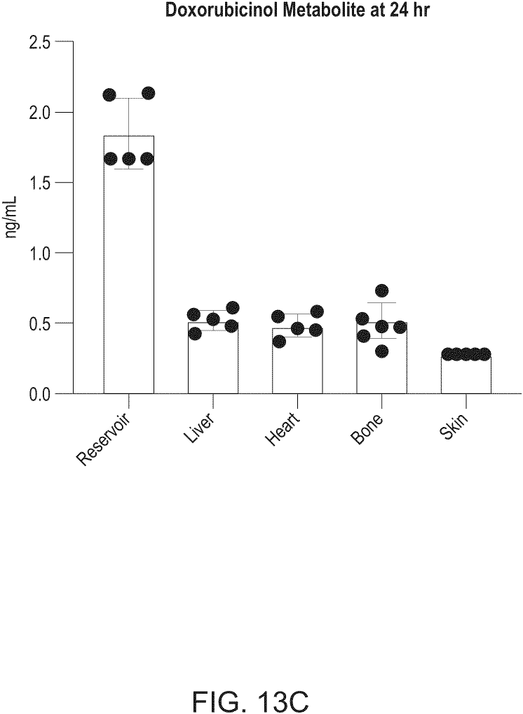

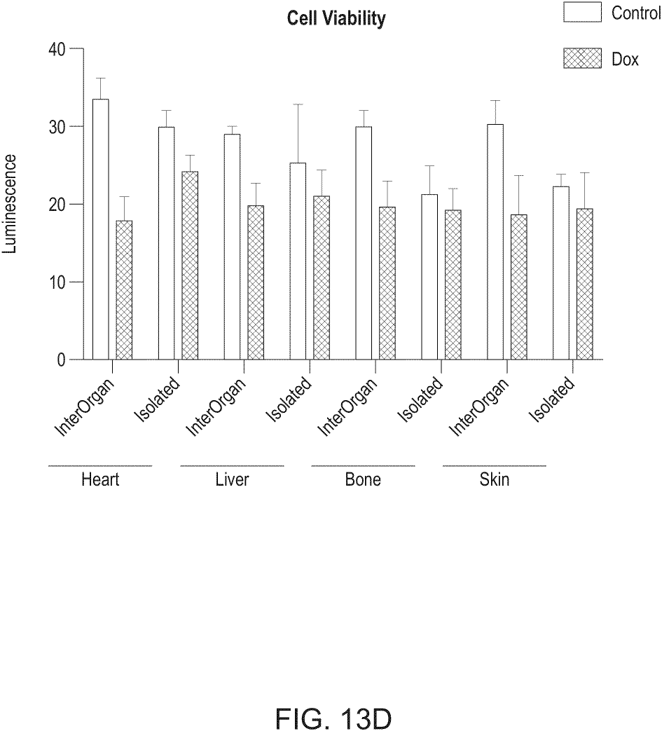

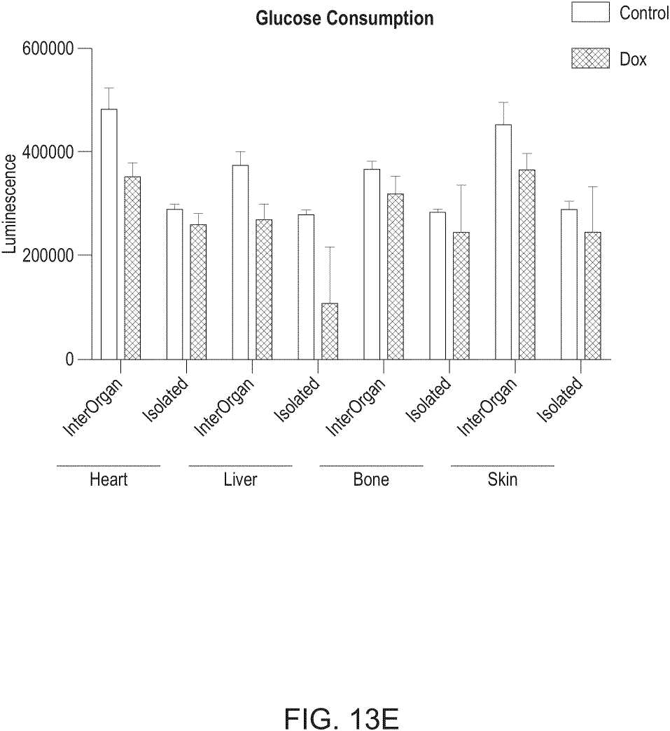

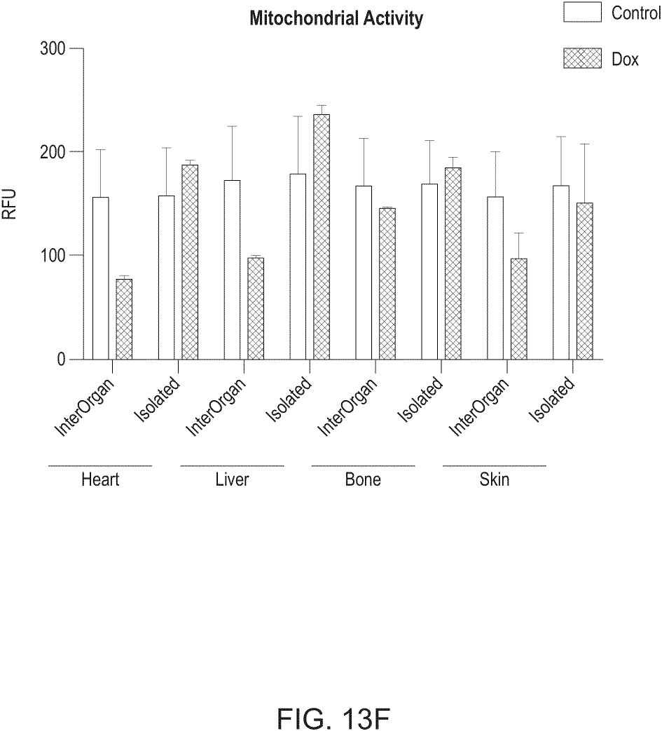

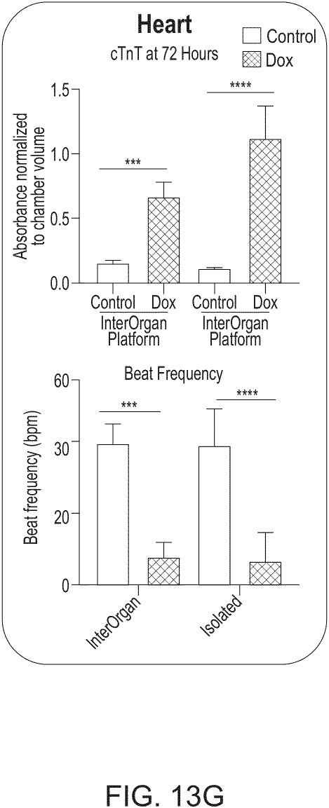

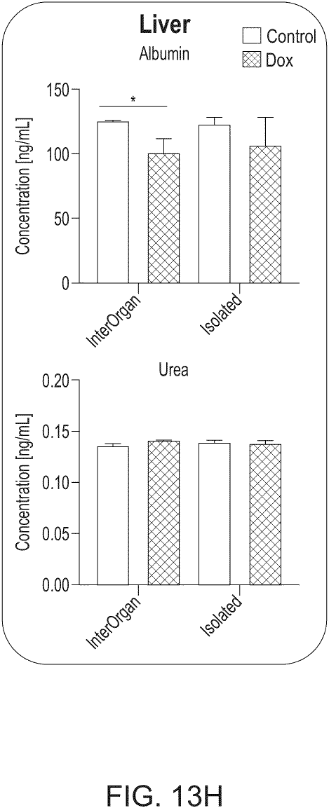

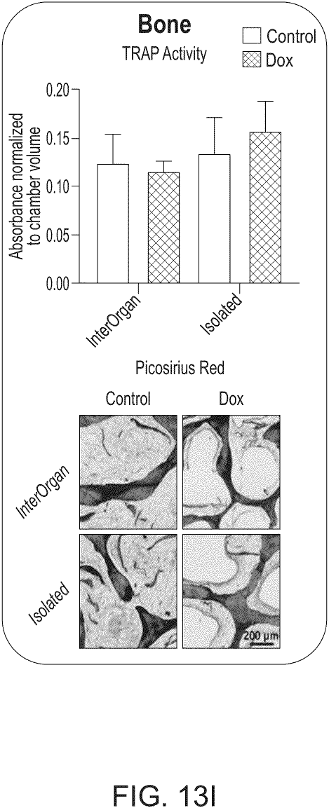

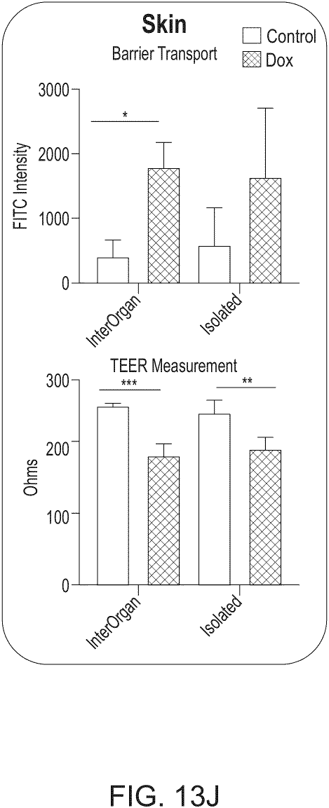

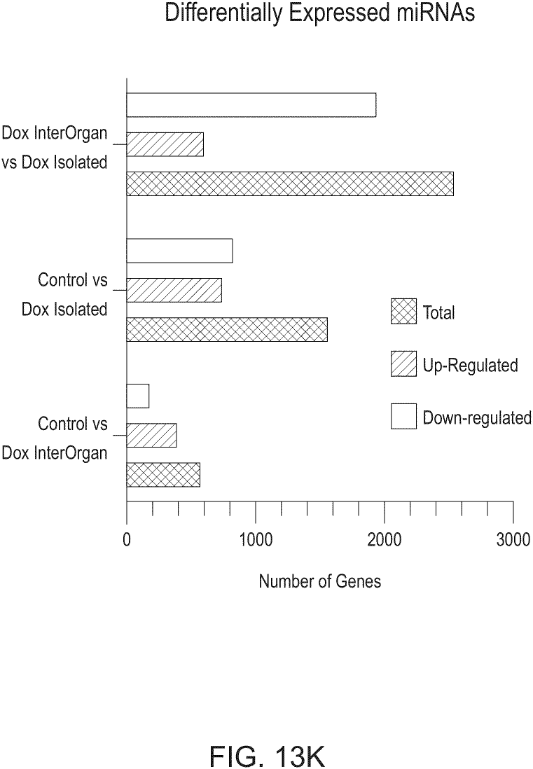

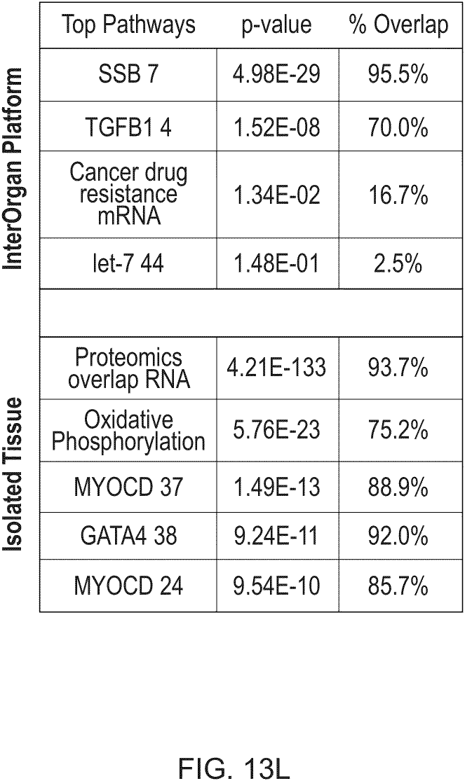

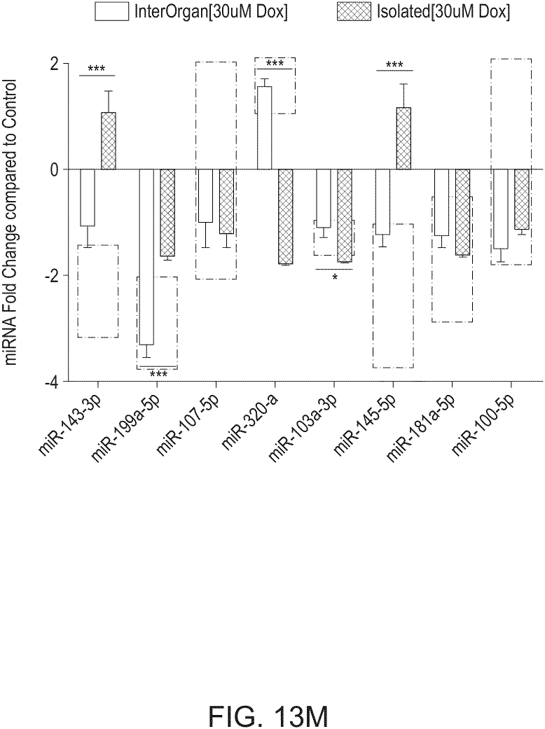

FIGS. 13A-M show that the InterOrgan platform reveals predictive off-target responses to doxorubicin. FIG. 13A shows the study design. FIG. 13B shows drug distribution in the reservoir over 72 hours (n=5). FIG. 13C shows a snapshot of doxorubicin metabolite production at 24 hours (n=5). After the acute dose, each engineered tissue was assessed for cell viability, glucose consumption, and mitochondrial activity 72 hours after exposure (FIGS. 13D-F). Functional features for each engineered tissue 72 hours after acute exposure (n=3-5) are shown in FIGS. 13G-J. Over a thousand miRNAs from control and Dox-exposed cardiac tissues were differentially expressed under Isolated and InterOrgan conditions (FIG. 13K). FIG. 13L shows pathways implicated in cardiotoxic function from isolated miRNAs. FIG. 13M shows that all identified miRNAs matched closely; miR-143-3p, miR-320a, and miR-145-5p are correctly predictive of patient cardiotoxicity as compared to the Isolated tissues (n=3). **p <0.01; ***p <0.001; ****p < 0.0001.

Treatment with 30 µM Dox, corresponding to the clinically administered cumulative dosage shown to induce cardiotoxicty, was delivered to tissues in the InterOrgan platform through the vascular channel and compared against the Isolated condition (FIG. 13A). Drug concentration in the perfusate decreased over time in the InterOrgan platform (FIG. 13B), along with the production of its metabolite (Doxorubicinol) via enzymatic breakdown in the liver tissue (FIG. 13C). Cell viability, glucose consumption, mitochondrial activity significantly decreased in all tissues in the InterOrgan platform (FIGS. 13D-F). We also detected decreased cardiac contractility and increased troponin release, both clinical measures of cardiac cell damage (FIG. 13G). Liver tissues showed decreased albumin and unaffected urea production (FIG. 13H), as seen clinically. Bone tissues showed stable TRAP responses but decreased cellularity (FIG. 13I), suggesting that osteoblasts are potentially more sensitive to Dox than osteoclasts, as suggested by pre-clinical studies. As expected, skin tissues showed increased diffusional permeability and decreased electrical resistance in response to Dox in both the Isolated and InterOrgan cultures (FIG. 13J).

We also evaluated miRNAs as early biomarkers of Dox cardiotoxicity, as suggested by recent clinical studies that identified 17 miRNA that were differentially expressed in pediatric cancer patients treated with Dox. We used the GeneChip™ miRNA 4.0 Array (ThermoFisher) to measure differential expression of miRNAs in heart tissues following Dox treatment. Differential miRNA expression in the InterOrgan platform more closely matched the published data (FIGS. 13K, M, 14B), suggesting that linking of matured tissues by vascular perfusion provides a more physiologically relevant context than tissues cultured in isolation.

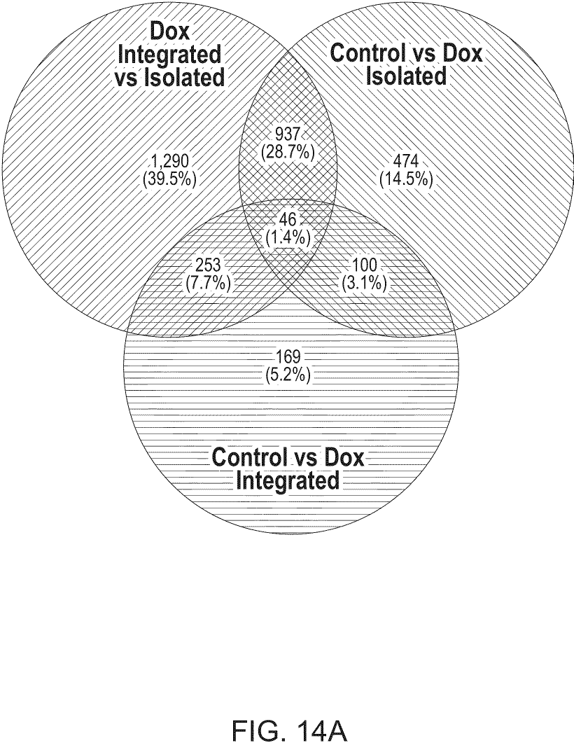

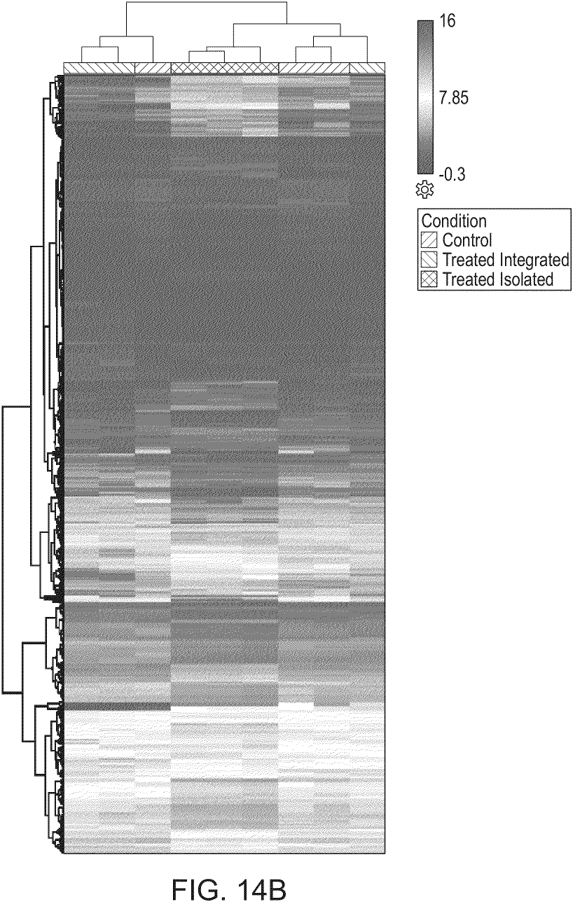

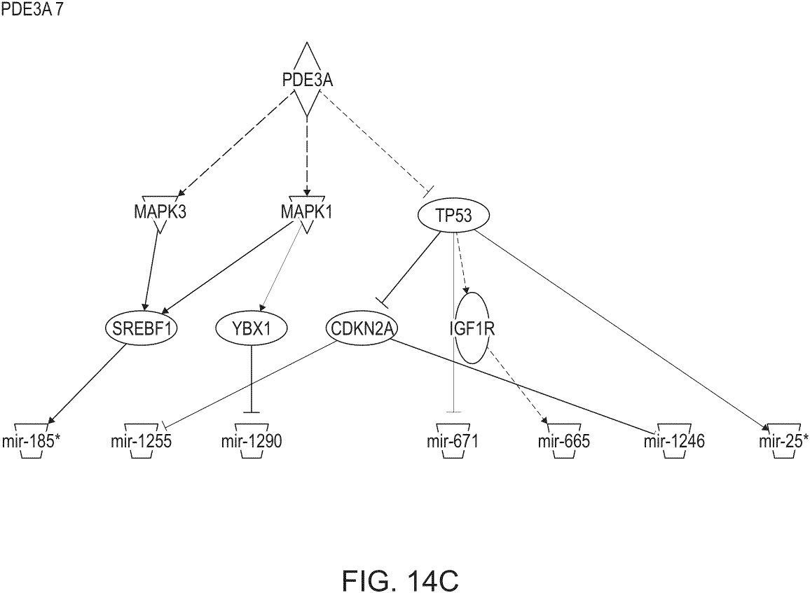

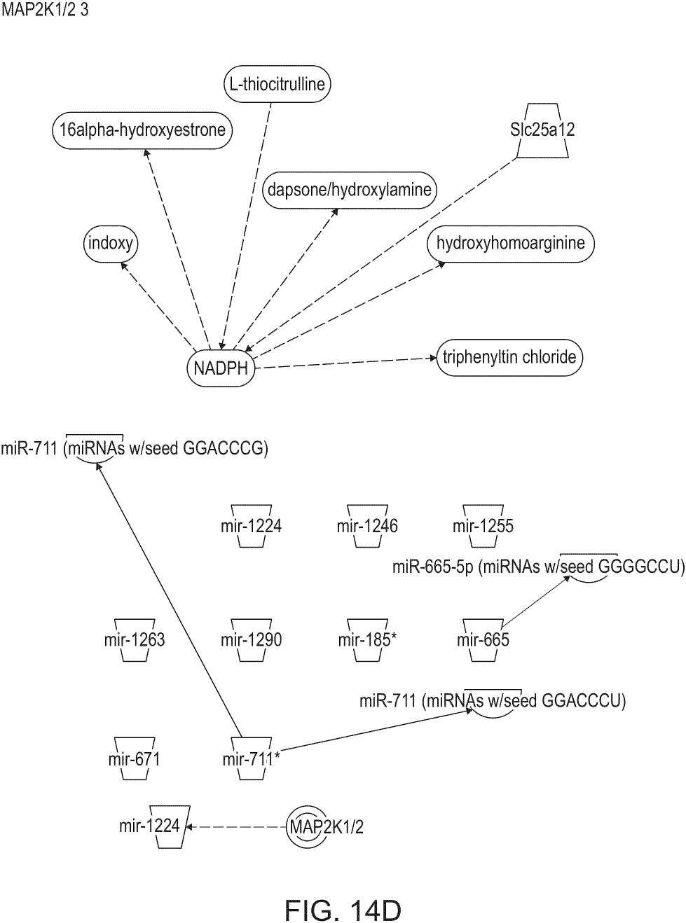

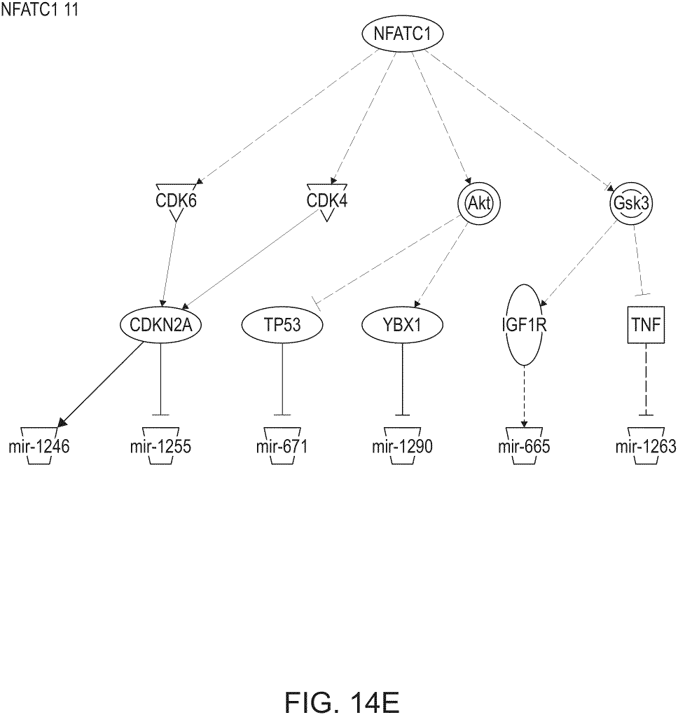

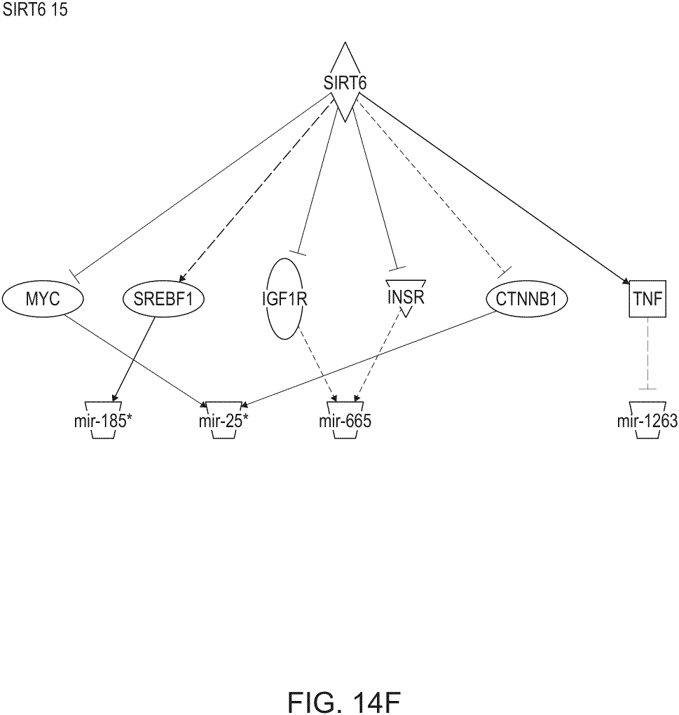

We then recapitulated the differential activities of miRNAs observed in pediatric study and clinical study in adults, by assessing enrichment of their repressed targets in differentially expressed genes, following Dox treatment. miR-6192 did not have enough target for enrichment analysis. Differential expressions of all other miRNAs were highly significant in the InterOrgan platform, based on Normalized Enrichment Scores (NES), showing activity consistent with published clinical results for 20 out of the 22 miRNAs (FIGS. 14).

FIGS. 14A-F show miRNA data for doxorubicin cardiotoxicity matched to patient data. FIG. 14A shows the number of cardiac tissue miRNAs among different conditions at t = 72 hours. FIG. 14B shows a heatmap demonstrating clustering of Dox-treated samples in integrated platform as compared to integrated controls (no drug) and Dox-treated isolated tissues. Doxorubicin-induced cardiotoxicity was matched to miRNA pathways regulating (FIGS. 14C,D) cardiac dysfunction and (FIGS. 14E,F) inflammation.

TABLE 2

Cardiotoxic functions found by miRNA pathway analysis for tissues cultured in integrated platform and in isolation

| | Cariotoxic Function | p-value Range | # of Molecules |
|---|---|---|---|
| IntOrgan Platform | Cardiac Dilation | 1.15E-42 - 1.15E-42 | 93 |
| | Cardiac Enlargement | 1.00E00 - 1.15E-42 | 107 |
| | Congenital Heart Anomaly | 1.00E00 - 8.39E-08 | 13 |
| | Cardiac Fibrosis | 3.74E-01 - 3.69E-07 | 28 |
| Islated Tissue | Cardiac Dilation | 5.02E-01 - 1.02E-22 | 195 |
| | Cardiac Enlargement | 6.49E-01 - 1.02E-22 | 372 |
| | Congenital Necrosis/Cell Death | 5.02E-01 - 1.52 E-16 | 158 |
| | Cardiac Arrythmia | 1.00E-00 - 4.17E-13 | 142 |

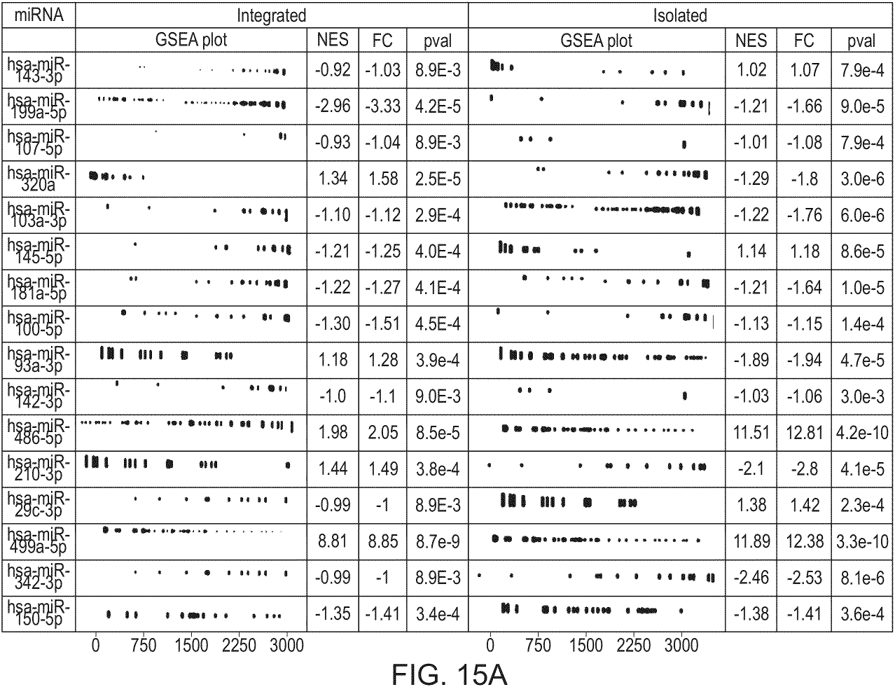

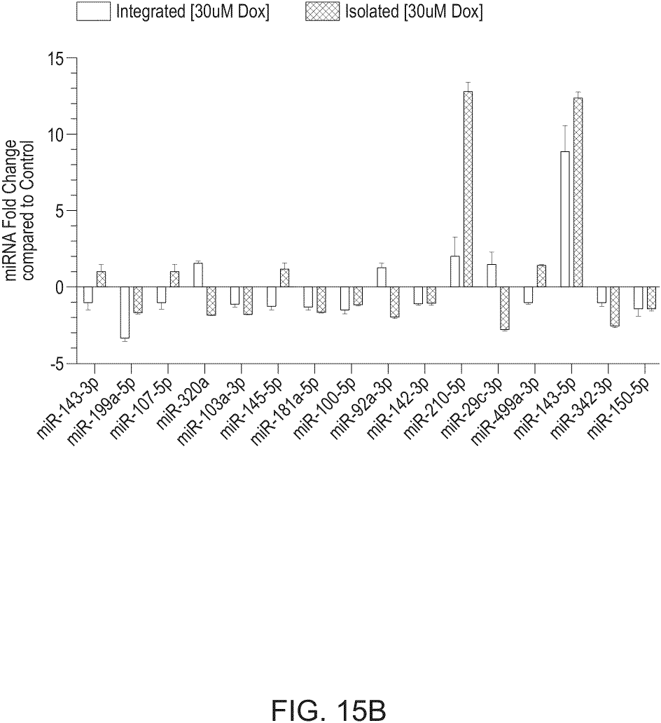

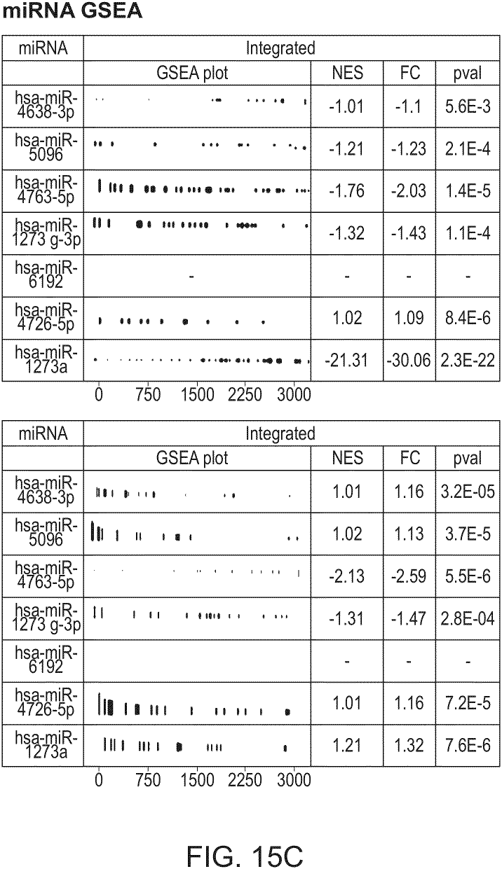

In contrast, while all miRNAs showed significant differences in the Isolated platform, only 12 were in agreement with published results, while 10 showed opposite differential activity (FIGS. 15A-C). FIGS. 15A-C show doxorubicin-induced cardiotoxicity matched to clinical benchmarks via gene set enrichment analysis (GSEA) (FIG. 15A) and fold change (FIG. 15B) of all miRNAs shown to be clinically relevant in pediatric patients. FIG. 15C shows GSEA of miRNAs shown to be clinically relevant in adult patients.

Indeed, in adult patients, miR-1273a was reported as a biomarker of high centrality for Doxorubicin-induced heart failure, with an “Energe” of -31.32. Consistently, dramatic reductions in the fold change and activity were detected in the InterOrgan platform (FC = -30.6, NES = -21.31, p = 2.3E-22), while the Isolated platform showed statistically significant opposite behavior, inconsistent with published data (FC = +1.32, NES +1.21). Taken together, these data show that the InterOrgan platform outperformed the Isolated culture, as evidenced by higher enrichment scores and fold changes consistent with those seen clinically (FIGS. 12M, 14). The utility of connecting multiple tissues to reproduce clinical predictions is further supported by the most differentially expressed miRNA in the InterOrgan data, hsa-miR-1273a.

Other cell types can be cultured in the platform, for example bone marrow models that include more cell types. This model may also be used to study a variety of different genetic blood disorders. Other applications include: Systemic disease modeling, cancer metastasis, human models of aging, human immune models/inflammation models, human fibrosis models, potency assays for biologics during drug development and manufacturing, bioreactor to grow engineered tissues/organs and cells for regenerative medicine, patient-specific avatars for patient centered modeling of health and disease risks/treatment valuation, human infection model, bioreactor for maintenance of engineered tissues, bioreactor for maturation of engineered tissues and microphysiological system for long-term disease modeling. The InterOrgan system could be used to physiologically mature tissue specific iPS cells for downstream use in regenerative medicine.

In summary, microphysiological systems containing human tissues bioengineered from iPS cells have long promised advantages towards modeling human physiology in vitro, as compared to cell monolayers and animal models. Although tissues in isolation can recapitulate some aspects of physiological function, studies of multi-organ, systemic interactions require communication of engineered tissues. The InterOrgan bioreactor system maintains the tissue and vascular niches that preserve mature tissue phenotypes and facilitate tissue communication by vascular perfusion, while separating the interstitial and intravascular compartments via a selectively permeable endothelial barrier. The platform recapitulated clinically observed off-target effects of several drugs, and enabled identification of early miRNA biomarkers of drug toxicity.

### Exemplary Materials and Methods

### Multi-Tissue System

The bioreactor system described herein, interchangeably referred to as “The InterOrgan platform” or “InterOrgan bioreactor system,” is designed to support the culture and communication of multiple types of engineered tissues. Each tissue is maintained within its own optimal medium and communication between tissues occurs via exchange to a recirculating shared vascular medium across an endothelium that serves as a selectively permeable barrier. The platform is sized to fit onto a standard glass microscope slide. In some embodiments, it includes four culture chambers that can each contain up to 1.5 mL of tissue specific medium, and a reservoir for access to the recirculating vascular medium. Two ports and a channel are included to enable recirculating flow of the shared vascular medium via a pump. A plate or a glass slide establishes the bottom boundary of the flow channel and enables real time imaging. Inserts for each chamber include a porous nylon mesh that serves as a substrate for the endothelial layer. The mesh pore size (20 µm) was selected to ensure the endothelial barrier is the primary regulator of exchange of various factors or cells.

System fabrication: The reservoir and flow channel component of the platform were fabricated from polysulfone (McMaster-Carr) using a 3-axis CNC milling machine (Haas OM2). The clamps and tubing transfer lid were machined in the same manner from polycarbonate (McMaster-Carr). The mesh barrier inserts were made via an overmolding process using an injection molding machine (A.B. Plastic Injectors, AB-200) and polypropylene thermoplastic (Flint Hills Resources P9M7R-056). An aluminum tool (alloy 7075, McMaster-Carr) was CNC machined for this process and a 20 um nylon net filter (Millipore) was laser cut (ULS VersaLaser 3.50) into 11 mm circles using a 30 W CO2 laser. The cut nylon filters were clamped into a multicavity aluminum tool, and polypropylene was injected to form the structure of the mesh insert. An o-ring was installed around the structure to provide a seal (Viton, dash 011, 60A durometer, McMaster-Carr).

The remaining components of the platform include: 100 mm cell culture dish, 1×25×75 mm glass slide, Pharmed tubing (1/16″ inner diameter, Cole-Parmer), peristaltic pump tubing (2.29 mm inner diameter, Pharmed, Cole-Parmer), 3-way stopcock valve (Smiths Medical ASD MX9311L), luer elbow connectors (Cole-Parmer EW-45501-84), and a peristaltic pump (Cole-Parmer EW-07557-00 and EW-07519-25). Connections between tubing components were made with appropriately sized barbed luer connectors (polypropylene, McMaster-Carr). Post-fabrication cleaning was done via ultrasonic cleaning and autoclaving on a wet cycle.

Platform assembly: Platform components were removed from sterile packaging in a biosafety cabinet and assembled sterily. A standard glass microscope slide was placed onto the bottom surface of the polysulfone reservoir flow array using a silicone o-ring. Two polycarbonate clamps are press-fit around the slide, silicone o-ring, and platform chambers to provide a compression seal. Luer elbow components are press-fit into luer-taper ports at the top of the platform. A loop of Phramed tubing was attached to a peristaltic section and 3-way valve, to connect the luer elbows at the inlet and outlet ports. The entire assembly was inserted into a 100 mm petri dish and a tubing-transfer lid was placed on top. Tubing is installed into the slots of the lid, while a standard lid is used to protect the platform from the above. With the tubing assembly completed, endothelial medium was infused via the 3-way valve to prime the flow loop and remove air. Once primed, the endothelialized mesh inserts were installed into each tissue well, and the assembly was then moved to an incubator and connected to a peristaltic pump.

Additional information about the components and assembly of the platform are in International Patent Application serial number PCT/US2019/43722, incorporated in its entirety herein.

### Study Design

System validation: The InterOrgan systems were autoclave-sterilized as individual components and then assembled in a biosafety cabinet. The platforms were primed via syringe with 12 mL of endothelial media through a luer-lock and 3-way stopcock valve. The vascular flow rates corresponded to hydrodynamic shear stresses of 1-5 dynes/cm², with potential to extend to 10 dynes/cm², if needed. Endothelial barrier was responsive to physiologic vasoactive agents such as thrombin. Platforms were connected to a peristaltic pump and operated at a shear stress of 1.88 dynes/cm². 1.5 mL of tissue specific media was added to the corresponding tissue chamber.

Engineered tissue designs: Cardiac tissues were formed from hiPSC-derived cardiomyocytes with supporting human fibroblasts in fibrin matrix stretched between two auxotonic flexible pillars, and electromechanically matured at an increasing intensity. Liver tissues were formed from aggregates of hiPSC-derived hepatocytes and supporting human fibroblasts that were encapsulated in fibrin hydrogel. Bone tissues were made by seeding human bone marrow-derived mesenchymal stromal cells (MSCs) in a decellularized bovine bone matrix scaffold and inducing the cells towards the osteoblastic phenotype; to recapitulate osteolytic bone, primary CD14⁺ monocytes were seeded into the osteoblastic bone and differentiated into osteoclasts, thereby forming mature bone with matrix secretion and resorption. Skin tissues were formed by seeding human dermal fibroblasts in layered 3D collagen matrix and then adding human keratinocytes onto the matrix. Skin tissues were cultured at air-liquid-interface to form matured stratified epidermis.

Platform validation over 4 weeks of culture: To validate the platform, we studied three different configurations: (1) “InterOrgan” system (n = 12), where the multi-chamber platforms contain endothelialized mesh inserts to separate tissue-specific niches from perfusion flow; (2) “Mixed″system, containing mesh inserts without endothelial barrier, allowing tissue and vascular culture media to mix rapidly, a condition equivalent to the use of common media for all tissues (n =6); and (3) “Isolated” system (n = 6), with each tissue cultured separately in the same volume of tissue-specific medium (~1.5 mL). Conditions (1) and (2) had a perfusate flow channel running on a peristaltic pump at hydrodynamic shear at the mesh of1.88 dynes/cm². Monocytes (50,000 CD14⁺ cells) were added into the reservoir for conditions (1) and (2) at t = 0 and 14 days; recirculating flow was maintained for 28 days. Every other day, 1 mL of medium was changed in each culture chamber and in the vascular reservoir; medium samples were immediately frozen at -20° C. for subsequent analysis. Similarly, 1 mL of media from Isolated tissues was taken and replenished every other day. At the end of the 4-week study, tissues were harvested and sectioned for proteomics (and immediately flash frozen), and histology (and immediately fixed in 4% paraform aldehyde).

Doxorubicin-induced toxicity (FIGS. 13): In order to compare the functional and molecular responses of tissues within our InterOrgan system (n =6 Dox; n =3 controls) to the responses of tissues cultured in isolation (n = 6; n = 3 Control), the InterOrgan platform and the isolated tissues were exposed to the same cumulative dose of Doxorubicin (30 µM).In the InterOrgan platform, we administered the drug into the reservoir compartment at t =0, and followed up for the 72 hours without media changes. Small media samples were collected from the reservoir at t = 5 min, 1 hour, 6 hours, 24 hours, and 72 hours for HPLC-MS quantification of nonmetabilized drug and metabolites. At 72 hours, the supernatant was collected, the functionality was assessed for all engineered tissues, and the cardiac tissues were sampled for evaluation of cardiotoxicity by miRNA isolation and analysis.

### Tissue Formation and Maturation

In addition to the tissues described below, one of ordinary skill in the art would appreciate that other tissues and fibroblasts may be used. For example, cardiac or iPS fibroblasts may be used instead of dermal fibroblasts. Further iPS derived versions of tissues are within the scope of the invention.

Heart: Cardiac tissues were formed and matured as described previously. Briefly, a ratio of 75% iPSC-derived cardiomyocytes were combined with 25% supporting normal human dermal fibroblasts (NHDF, Lonza) in 84 µL of 33.3 mg/mL human fibrinogen (Sigma-Aldrich, F3879) and crosslinked with 16 µL of 100 U/mL thrombin from human plasma (Sigma -Aldrich, T6884) to form a hydrogel between two flexible pillars. After 20 minutes of crosslinking at 37° C. in 5% CO₂, cardiac media was added (RPMI 1640 (Thermo Fisher Scientific, 11875-093), B27 supplement (serum free, Thermo Fisher Scientific, 17504044), ascorbic acid (Sigma-Aldrich, A8960) and penicillin/streptomycin (Gibco by Life Technologies, 15070063)) supplemented with 0.02 mg/mL of aprotinin (Sigma-Aldrich, A3428). After 1 week of compaction, cardiac tissues were transferred to the maturation platform where they were subjected to electromechanical conditioning at a frequency increasing from 2 Hz to 6 Hz (biphasic stimulation, 2 ms pulse duration, 4.5 V/cm field intensity).

Liver: Human iPSC-derived hepatocytes were purchased from Cellular Dynamics International (CDI, iCell Hepatocytes 2.0) and thawed at room temperature. An AggreWell plate with 400 µm microwells (STEMCELL Technologies) was prepared according to the manufacture’s protocol. Hepatocytes (10 million cells) were mixed with NHDF (10 million cells) in hepatocyte culture medium (HCM, Lonza). The dual cell suspension was then added to 20 wells (approximately 500,000 hepatocytes and 500,000 NHDF in each well) with 2 mL of HCM per well). After 48 hours of culture at 37° C. and 5% CO₂, the formed cell aggregates were harvested and encapsulated in a fibrin hydrogel formed from fibrinogen (84% of total volume) and thrombin (16%). The cells in hydrogel were placed into 48-well tissue culture-treated cell culture plates (Corning), using 200 µL per well. The hydrogel was allowed to cross-link in a cell culture incubator for 20 minutes, after which 1 mL hepatocyte media supplemented with 1 mg/mL of aprotinin were added. Tissues were allowed to polymerase for at least 12 hours before being used in experiments.

Bone: Bovine calf metacarpals were purchased in bulk and stored at -40° C. (Lampire Biological Laboratories, #19D24003). A band saw was used to cut approximately 4 cm tall trabecular bone sections from the distal end of metacarpal. A CNC Milling machine was then used to generate bone cores with a cross section of 4 mm × 8 mm that were cut into 1 mm thick sections using an IsoMet low speed watering saw. Each section (4 mm wide × 8 mm long × 1 mm thick), was decellularized using our previously established protocols, to remove all cellular material while preserving the bone matrix composition and architecture. Bone scaffolds were processed in batch by following the following step-wise protocol on an orbital shaker: (i) PBS with 0.1 % EDTA (w/v) for 1 hour at room temperature; (ii) 10 mM tris, 0.1% EDTA (w/v) in DI water overnight at 4° C.; (iii) 10 mM Tris, 0.5% sodium dodecyl sulfate (w/v) in DI water for 24 hours at room temperature; (iv) 100 U/ml DNase, 1 U/ml RNase, 10 mM Tris in DI water for 6 hours at 37° C. The resulting bone matrix scaffolds were lyophilized and weighed to ensure that each piece had an appropriate matrix density for cell seeding (12 - 18 mg per scaffold). For sterilization, bone scaffolds were subjected to 70% ethanol treatment overnight under ultraviolet light, and then incubated in DMEM overnight.

Bone-marrow derived MSCs (Lonza) were expanded and seeded into the bone matrix scaffolds using 4 × 10⁵ cells per scaffold suspended in 40 µL of medium (DMEM supplemented with 10% (v/v) HyClone FBS, 1% penicillin/streptomycin, and 1 ng/mL of basic fibroblast growth factor b, bFGF), according to established protocols [8]). The cells were allowed to attach for 2 hours, and then supplemented with additional medium (DMEM supplemented with 10% (v/v) HyClone FBS, 1% penicillin/streptomycin, and 1 ng/mL of basic fibroblast growth factor b, bFGF) overnight. The following day, osteogenic differentiation was initiated by changing the media to osteogenic media consisting of low glucose DMEM supplemented with 1 µm dexamethasone (Sigma Aldrich), 10 mm β-glycerophosphate (Sigma Aldrich), and 50 µm L-ascorbic acid-2-phosphate (Sigma Aldrich). Each scaffold was incubated in 4 mL of osteogenic media, with media changes 3 times a week, for 3 weeks, allowing for the MSCs to differentiate into functional, maturing osteoblasts.

Primary monocytes were expanded, seeded into the bone scaffolds, and differentiated into functional, mature osteoclasts using our previously developed protocols. Briefly, peripheral blood mononuclear cells (PBMC) were isolated from buffy coats of human blood (fully deidentified samples obtained from the New York Blood Center) by density gradient centrifugation with Ficoll-Paque PLUS (GE Healthcare, 17-1440-02). Following manufacturer’s protocol, immunomagnetic isolation of monocytes (Big Easy EasySep Magnet, Stem Cell Technologies, 180001) using negative selection (EasySep Human Monocyte Isolation Kit, Stem Cell Technologies, 19359) was performed. For the following 2 days, 8 × 10⁶ monocytes were cultured on 25 cm² ultralow attachment flasks (Corning, 3815) with 10 mL of maintenance medium (RPMI 1640, ATCC, 30-2001) supplemented with 10% heat-inactivated human serum (Corning, 35-060), 1% penicillin/streptomycin, and 20 ng/mL Recombinant Human M-CSF (PeproTech, 300-25) at 37° C. in a humidified incubator at 5% CO₂. Human CD14⁺ monocytes were then seeded at a concentration of 4 × 10⁵ cells per scaffold, using 40 µL of medium, allowed to attach for 2 hours at 37° C. in a humidified incubator at 5% CO2, and subsequently differentiated for 2 weeks into osteoclasts in Minimum Essential Medium Eagle Alpha modification (α-MEM, Sigma, M4526) supplemented with 10% (v/v) heat-inactivated HyClone FBS, 1% penicillin/streptomycin, 1-Glutamine (Gibco, 25030-081), 20 ng/mL Recombinant Human M-CSF (PeproTech, 300-25) and 40 ng/mL Recombinant Human sRANK Ligand (PeproTech, 310-01). Cytokines were replenished every 3 days. Cells were maintained at 37° C. in a humidified incubator at 5% CO₂.

Skin: 3D skin tissues were formed following a previously described protocol. Briefly, 1.5 × 10⁵ human dermal fibroblasts (NHDF)n were embedded in 1 mL of 3 mg/mL type I collagen matrix (Millipore, 08-115) and the polymerized cell-containing gel was incubated on a transwell mesh (BD Biosciences) for 5 to 7 days in Dulbecco’s Modified Eagle Medium (DMEM) supplemented with 10% FBS. Then, 2.5 × 10⁵ keratinocytes were seeded onto the matrix, and incubated in epidermalization medium containing a 3:1 mixture of DMEM and HAM’S F12, 0.1 % FBS, 2 nM triiodothyronine (T3) (Sigma, T5516), 5 ng/ml insulin (Sigma, I9278), 0.4 µg/ml hydrocortisone (Sigma, H0888) and 10 ng/ml EGF (Millipore, 01-107) for an additional 6 days to ensure keratinocytes were confluent enough to cover the surface. The composite culture was raised to the air-liquid interface for 7 days in a cornification medium with high calcium concentration (1.8 mM), without growth factors, to induce epidermal differentiation.

Endothelial barriers: Custom mesh inserts were fabricated as described above, autoclaved on a wet cycle, coated with fibronectin (1:100 from 10 ug/mL stock, Sigma, F4759) for 45 minutes and washed twice with PBS. Human MSCs were expanded in monolayers and dissociated with trypsin between passage 5 and 8, and were then seeded using 150,000 cells in 50 µL volume to the top of each insert. The MSC cell suspension (50 µL) was left on each mesh insert for 1 hour to enable cell attachment. After 1 hour, additional media was added to each well (2 mL/well), fully immersing the cell coated meshes within the wells of the ultra-low attachment plate (Corning, 3473), and cultured at 37° C. with 5 % CO₂ for 24 hours. Human umbilical venous endothelial cells (HUVEC) were expanded to passage 5 - 8. The bottom surface of each MSC-coated inset was coated by 400,000 HUVEC and an additional 50,000 MSC. To this end, the MSC media was removed, allowing for each insert to stay only slightly hydrated and flipped over to the bottom side. A 20 µL cell suspension of HUVEC/MSC was added twice, 15 minutes apart, to the bottom surface of each insert and incubated at 37° C. and 5% CO₂ in-between the two cell additions, allowing for incremental attachment of cells prior to addition of endothelial media (EGM-2, Lonza). Each insert was estimated to have a total of 400,000 HUVECs and 200,000 MSCs, to mimic the dynamics between vascular populations, represented by the endothelium and perivascular supporting cells (pericytes) in blood vessels. After 48 hours, mesh inserts with adherent cells were placed into the platforms, and exposed to hydrodynamic shear stress of 0.31 dynes/cm² for 12 hours, 0.63 dynes/cm² for 24 hours, and 1.88 dynes/cm² for 24 hours. To validate the barrier function of these endothelial/MSC coatings, FITC/TRITC-tagged 3 kDa and 70 kDa Dextran molecules (ThermoFisher, D3307, D1864) were flowed through the platforms for 72 hours and collected from the top chambers to read their fluorescent output using a 96-well plate reader (BioTex, Synergy HTX).

Immune cells: Primary human CD14⁺ monocytes were isolated by using magnetic activated cell sorting (MACS) using CD14⁺ sorting beads (Miltenyi Biotec) from a human leukopak sample (New York Blood Center). The isolated cells were maintained in a buffer solution on ice for less than 3 hours prior to introduction into the platform. CD14⁺ monocytes (50,000 cells) were introduced into the vascular perfusion reservoir and circulated through the platform for the duration of the experiment. At day 14, monocytes were replenished by introducing an additional 50,000 CD14⁺ cells into circulation through the reservoir.

Cardiac cryoinjury studies: Vascularized InterOrgan platforms were assembled as described above, and each platform contained only cardiac tissues in the middle two chambers (first and last chamber remained empty). One of the two cardiac tissues was then exposed to cryoinjury by touching the tissue with dry ice for 5 seconds, while the other served as a control. Immune cells (200,000 CD14⁺ monocytes) were labeled with Vybrant™ DiD Cell-Labeling Solution (ThermoFisher) to enable tracking over time, introduced into the reservoir and circulated in the platform for the duration of the experiment. The platforms were maintained for 7 days without media change, and the cardiac tissues were imaged with an IVIS Spectrum Optical Imaging System (Perkin-Elmer), in the Columbia’s Oncology Precision Therapeutics and Imaging Core (OPTIC). For imaging, the cardiac tissues were removed from the InterOrgan platform to avoid autoflourescent signals. The healthy and injured tissues were aligned next to one another in the same field of view and compared directly from multiple imaging views (top, side) using an IVIS 200 Spectrum device. The IVIS software was used to analyze the images by converting the signal to the normalized Radiant Efficiency (Emission light [photons/sec/cm²/str]/ Excitation light [µW/cm²]). The fluorescence signal was measured by selecting the same region of interest for each tissue and subsequently quantifying the sum of the Radiant Efficiency of all fluorescent pixels within the region of interest. The results were graphed using GraphPad prism. Exported images showing the Radiant Efficiency as a heat map were generated within the IVIS Spectrum software (Perkin-Elmer).

### Supernatant and Functional Assays

Supernatant collection: Every 2 days, supernatant samples (1 mL volume) were collected from each chamber and reservoir, frozen immediately, and subsequently thawed and used for several assays as described below. Supernatant samples were stored at -20° C. for less than three months before use; once thawed, no supernatant sample was left at 4° C. for more than one week and thawed no more than 2 times.

Cytokine profiles: MIP3a and SDF1a readings were obtained from 50 µL of tissue supernatant taken after 28 days of culture using the Immune Monitoring 65-Plex Human ProcartaPlex™ Panel (Thermo Fisher Scientific, EPX650-10065-901), according to the manufacturer’s instructions. Samples were allowed to incubate overnight at 4° C., run on a Luminex-200 and analyzed through the Luminex software by comparing to the included standards.

Heart function assays: Cardiac excitability, force, and beat rate were obtained using our previously established protocols. Cardiac troponin secretion was determined using the Human Cardiac Troponin I ELISA Kit (Abcam, ab200016) according to the manufacturer’s instructions.

Liver function assays: To assess liver function, supernatant samples from each condition at the beginning and after 28 days of culture were analyzed for albumin and urea secretion using a Human ELISA kit (Bethyl, E88-129) and a Urea Nitrogen Test Kit (Fisher Scientific, SB-0580-250), respectively. Comparing these samples to the provided standard, concentrations of albumin and urea were calculated and compared to the secretion for each tissue at Day 0. Assays were performed according to the manufacturer’s instructions.

Bone function assays: To assess the bone’s ability to remodel its matrix, supernatant samples were analyzed for Telomer Repiceated Amplification Protocol, (TRAP,, Kamiya Biomedical Company, KT-008) and bone sialoprotein (Human Bone Sialoprotein ELISA Kit, Mybiosource, cat. no. MBS261861) after 28 days of culture. These assays were performed according to the manufacturer’s instructions.

Skin functional assays: For drug-induced toxicity studies, electrical current was run through the tissues by placing electrodes on either side of the skin tissue and the change in resistance was subsequently measured and read as a function of the barrier. All tissues were analyzed using the same electrode holder setup to standardize the positions of electrodes with respect to the tissue. To analyze the transport of molecules through the skin barrier, Fluorescein (FITC, ThermoFisher, D3306) powder was added to the epidermis of skin tissues. After 3 hrs, 100 µl of supernatant from the dermal region below the skin tissue was sampled and assayed on a plate reader (BioTex Synergy HTX).

Metabolic Assays: Glucose, lactate, glutamate and glutamine were measured in parallel using the bioluminescent Glucose-Glo™ (Promega, cat. no. J6021), Lactate-Glo™ (Promega, cat. No. J5021), Glutamine/Glutamate-Glo™ (Promega, cat. no. J8021) and Glutamate-Glo™ (Promega, cat. no. J7021) assays according to the manufacturer’s protocols. Supernatants were thawed and 2.5 µl of sample was diluted in 97.5 µl PBS and the following volumes were used from this mixture for each assay: 25 µl for lactate, 12.5 µl plus an additional 12.5 µl PBS for glucose, 12.5 µl for glutamine and 12.5 µl for glutamate, as suggested by the manufacturer.

### End Point Assays

Tissue preparation: Collected tissue samples were bisected for proteomic and histologic analyses. One half of the sample was snap frozen using liquid nitrogen and stored at -80° C. for less than one month before being analyzed for proteomics. The second half of each tissue sample was fixed for 24 hours in PFA, washed in PBS, and submitted to the Herbert Irving Comprehensive Cancer Center (HICCC) Molecular Pathology Lab at Columbia University for paraffin-embedding and sectioning.

Immunostaining: Heart, bone, liver, and skin constructs were fixed in 4 % PFA for 24 hours, embedded in paraffin, and sectioned for histological and immunofluorescence examination at 5 µm. All tissues were processed for hematoxylin & eosin (H&E), trichrome, and bone tissues were processed for picrosirius red staining by the HICCC Molecular Pathology Lab at Columbia University. Paraffin-embedded tissue blanks were hydrated, processed for antigen-retrieval using a 10 mM sodium citrate buffer for 20 min in heat, and permeabilized with 0.25% (v/v) Triton-X for 20 minutes. Samples were then blocked for 2 hours with 10% FBS, and individual staining protocols for each tissue. Heart: Samples were incubated with a primary antibody for alpha-actinin-2 (Invitrogen, 701914) overnight at 4° C. Liver: Samples were incubated with primary antibodies for albumin and cytochrome P450 enzyme CYP3A4 (Millipore, AB1254) overnight at 4° C. Skin: Samples were incubated with primary antibodies for keratin 14 (Biolegend, PRB-155P) and vimentin (Santa Cruz Biotechnology, sc-6260) overnight at 4° C. After washing with PBS, samples were incubated with fluorophore-conjugated secondary antibodies (Invitrogen) for 2 hours at room temperature. Slides were covered with cover-slips using mounting medium containing 4′,6-diamidino-2-phenylindole (DAPI) (Prolong Mountant with NucBlue, Invitrogen, P36981) and examined using either a Zeiss LSM 5 Exciter confocal laser scanning microscope or Nikon Ti Eclipse inverted confocal microscope.

Endothelial layers were fixed in PFA at 37° C. for 10 minutes. After aspirating fixation solution, samples were washed delicately using PBS supplemented with 1 mM CaCl₂ and 0.5 mM MgCl₂. Samples were subsequently permeabilized using 0.5 % Triton X-100 at 37° C. for 10 minutes. Once washed using supplemented PBS, endothelial barriers were stored at 4° C. for less than three weeks prior to staining. For staining, a dilution of 1:250 for VE-Cadherin (Sino Biological, 10433-MM01) in 2 % BSA was added to each sample at 4° C. overnight. Samples were subsequently washed with supplemented PBS three times for 5 minutes each. For secondary staining, 1:400 488 Goat anti-mouse IgG in 2 % BSA, 1:400 Phalloidin, and 1:1000 DAPI were added to each endothelial barrier sample. Samples were kept in the dark on a shaker overnight and washed three times for 5 minutes each the next day prior to imaging.

Quantitative Proteomics: Proteomics sample preparation and tandem mass tag (TMT) labeling were performed as described earlier (1), with minor modifications. Briefly, frozen tissues were lysed by bead-beating in 8 M urea, 1% SDS, 200 mM EPPS (pH 8.5) and protease inhibitor. Samples were reduced with 5 mM TCEP and alkylated with 10 mM iodoacetamide (IAA) that was quenched with 10 mM DTT. A total of 50 µg of protein was chloroform-methanol precipitated. Protein pellets were reconstituted in 200 mM EEPS (pH 8.5) and protein concentration determined using a BCA assay (Pierce). Total protein from each sample (2 to 25 µg) was digested overnight at room temperature with Lys-C protease at a 50:1 protein-to-protease ratio while shaking. Trypsin was then added at a 100:1 protein-to protease ratio, and the reaction was incubated 6 hours at 37° C. Digested peptides were quantified using a Nanodrop at 280 nm and 2 to 25 µg of peptide from each sample were labeled with 200 µg TMT reagent using 10-plex TMT kit. TMT labels were checked by pooling 100 ng of each sample and were bulk mixed at 1:1 across all channels using normalization factor samples. Bulk samples were fractionated with using Pierce™ High pH Reversed-Phase Peptide Fractionation Kit and each fraction was dried down in a speed-vac. Dried peptides were dissolved in 10 µl of 3% acetonitrile/ 0.1% formic acid and injected using SPS-MS3.

LC-MS/MS proteomics: Fractioned peptides were separated using Thermo Scientific™ UltiMate™ 3000 RSLCnano system and Thermo Scientific EASY Spray™ source with Thermo Scientific™ Acclaim™ PepMap™100 2 cm × 75 µm trap column and Thermo Scientific™ EASY-Spray™ PepMap™ RSLC C18. 50 cm × 75 µm ID column with a 5-30% acetonitrile gradient in 0.1% formic acid over 127 min at a flow rate of 250 nL/min. After each gradient, the column was washed with 90% buffer B for 5 min and re-equilibrated with 98% buffer A (0.1% formic acid, 100% HPLC-grade water) for 40 minutes. For BPRP-separated proteome fractions, the full MS spectra were acquired in the Orbitrap at a resolution of 120,000. The 10 most intense MS1 ions were selected for MS2 analysis. The isolation width was set at 0.7 Da and isolated precursors were fragmented by CID at a normalized collision energy (NCE) of 35 % and analyzed in the ion trap using “turbo” scan speed. Following acquisition of each MS2 spectrum, a synchronous precursor selection (SPS) MS3 scan was collected on the top 10 most intense ions in the MS2 spectrum. SPS-MS3 precursors were fragmented by higher energy collision-induced dissociation (HCD) at an NCE of 65% and analyzed using the Orbitrap.

Proteomic data analysis: Raw mass spectrometric data were analyzed using Proteome Discoverer 2.2 to perform database search and TMT reporter ions quantification. TMT tags on lysine residues and peptide N termini (+229.163 Da) and the carbamidomethylation of cysteine residues (+57.021 Da) was set as static modifications, while the oxidation of methionine residues (+15.995 Da) and deamidation (+0.984) on asparagine and glutamine were set as a variable modification. Data were searched against UniProt Human database with peptide-spectrum match (PSMs) and protein-level FDR at 1% FDR. The signal-to-noise (S/N) measurements of each protein were normalized so that the sum of the signal for all proteins in each channel was constant, to account for equal protein loading. Protein identification and quantification were imported into Perseus for multiple-sample tests for statistical analysis (FDR<0.05 or FDR<0.01) to identify proteins demonstrating statistically significant changes in abundance.

Proteomic data benchmarking of engineered against adult tissues: The identified proteins demonstrating statistically significant changes in abundance were compared to published adult tissue datasets from the Human Protein Atlas, as follows. Because the methodology used to generate each dataset varies greatly, direct comparisons could not be made. Instead, within each tissue dataset, individual protein expression levels were exported into Excel and subsequently tertiled into “Low”, “Medium”, and “High” expression levels, as is done in the Human Protein Atlas. Proteins that were not expressed were labelled as “Not detected” to avoid skewing comparisons with false low counts.

The percent of shared proteins was calculated by determining the number of proteins expressed (at any level) in the engineered tissue dataset versus the published adult tissue dataset, as a percentage of shared proteins (expressed in both tissues) within the total number of proteins. To further determine the correlation of the shared protein expression levels between the engineered tissues and the corresponding published data, the number of matching genes expressed as “Low”, “Medium”, or “High” in both tissue sets were calculated as a percentage over the total number of shared proteins expressed overall.

Heatmaps were generated using the tertiled data, by manually selecting a list of proteins according to two criteria: (1) the protein should be considered “tissue enriched” by the Human Protein Atlas and (2) the protein should be expressed in both datasets to enable comparisons. The lists of proteins for each heatmap were generated using only the proteins listed as highly expressed within each tissue per Human Protein Atlas. However, when the suggested protein was not expressed in one of the datasets, we continued down the list until finding data for 15 proteins per tissue. Comparisons between engineered and adult tissues were made according to tissue type, with engineered bone notably lacking a proper comparison. Bone is deemed a “rare” tissue by the Human Protein Atlas, and further literature searches did not yield more closely matching datasets, therefore the engineered bone protein data was compared to adult bone marrow protein data, the closest tissue comparison.

Proteomic data gene ontology (GO), KEGG, differential expression and pathway analysis: The identified proteins demonstrating statistically significant changes in abundance were subsequently used to perform gene ontology (GO) and KEGG pathway analyses as follows. GO analysis of shared highly expressed proteins in all tissues within each culture condition (InterOrgan, Mixed, Isolated) was performed using ShinyGO v0.61. First, gene lists were assembled using Microsoft Excel by filtering for genes (as determined from the corresponding proteins in the description corresponding to each protein accession number) listed as highly expressed in the heart, liver, skin, and bone tissues, according to the previous methodology where each data set was tertiled within Excel. This list was uploaded to the ShinyGO v0.61 server with the following settings: “Human” as the “Best matching species”, “0.05” as the “P-value cutoff (FDR)”, and “30” as the “# of most significant terms to show”. The resulting networks were directly exported from the site and used without editing. Tissue specific GO analysis was conducted using PANTHER (Protein Analysis Through Evolutionary Relationships) Classification System using lists in Excel, by filtering for genes (determined from the corresponding proteins in the description corresponding to each protein accession number) listed as highly expressed in the engineered tissue (e.g., heart) for each of the culture conditions (InterOrgan, Mixed, Isolated). The input genes were compared against all genes within the Homo sapiens reference list and a PANTHER Overrepresentation Test for GO cellular component complete was performed using a FISHER test with FDR correction. The resulting lists were exported, and GraphPad Prism software was used to graph cellular components associated with cardiac maturity. Further expression analysis for engineered tissues was performed using iDEP (integrated Differential Expression and Pathway analysis), where the protein expression datasets generated for each engineered tissue were uploaded and pathway analysis was performed, using the Human reference dataset, using PGSEA on for GO Biological Process and KEGG pathways for the top 30 pathways with a pathway significance cutoff (FDR) of 0.2 and a geneset minimum of 15 and maximum of 2000.

RNA sequencing: Cardiac tissues were flash frozen in RNAlater (ThermoFisher, AM7021) and sent to GENEWIZ for Standard RNA-seq with polyA selection using an Illumina HiSeq, 2×150bp configuration, single index, per lane and subsequent analysis as described below. Sequence reads were trimmed to remove possible adapter sequences and nucleotides with poor quality using Trimmomatic v.0.36. The trimmed reads were mapped to the Homo sapiens GRCh38 reference genome available on ENSEMBL using the STAR aligner v.2.5.2b. The STAR aligner is a splice aligner that detects splice junctions and incorporates them to help align the entire read sequences. BAM files were generated as a result of this step. Unique gene hit counts were calculated by using feature Counts from the Subread package v.1.5.2. Only unique reads that fell within exon regions were counted. After extraction of gene hit counts, the gene hit counts table was used for downstream differential expression analysis. Using DESeq2, a comparison of gene expression between the Control tissues and Matured tissues was performed. The Wald test was used to generate p-values and log2 fold changes. Genes with a p-value < 0.05 and absolute log2 fold change >1 were called as differentially expressed genes for each comparison. The differentially expressed genes bi-clustering heat map was generated to visualize the expression profile of the top 30 genes sorted by their adjusted p-values. This analysis was useful for identifying co-regulated genes across the treatment conditions. A second plot was generated to include only the top statistically significant differentially expressed genes if two or more were identified. This Volcano plot shows the global transcriptional change across the groups compared. All the genes are plotted and each data point represents a gene. The log2 fold change of each gene is represented on the x-axis and the log10 of its p-value is on the y-axis. Genes with a p-value less than 0.05 and a log2 fold change greater than 1 are indicated by red dots. These represent upregulated genes. Genes with a p-value less than 0.05 and a log2 fold change less than -1 are indicated by blue dots. These represent downregulated genes.

Ingenuity Pathway Analysis: Data were analyzed through the use of IPA (QIAGEN Inc., https://www.qiagenbioinformatics.com/products/ingenuitypathway-analysis).

Networks, functional analyses, and canonical pathways were exported directly and used without further modification.

### Drug Studies

Cardiac dose response studies: Cardiac tissues and strips of fetal cardiac tissue (FCT, purchased as surgical waste from Advanced Bioscience Resources (Alameda, CA)) were imaged at baseline and at each sequential dosage for 20 seconds at 100 frames per second under brightfield illumination using a Zyla 4.2 sCMOS camera (Andor) and NIS software (Nikon) or Pike F-032b (Allied Vision Technologies) camera. The resulting videos were analyzed for pixel movement in a custom MATLAB code described previously.

Responses to epinephrine, dofetilide, doxorubicin, and doxorubicinol: The comcentrations of epinephrine, dofetilide, doxorubicin, and doxorubicinol were measured in cell culture supernatant using ultra performance Liquid Chromatography-tandem Mass Spectrometry (UPLC-MSMS). For epinephrine, samples were spiked with the internal standard (norepinephrine-d6), and mixed with 1.2 M perchloric acid, followed by 1 M sodium bicarbonate. After mixing, 0.1% dansyl chloride was added, vortexed and heated at 60° C. for 10 minutes. After being chilled on ice for 3 minutes, the samples were centrifuged at 13,000 rpm for 5 minutes. The supernatant was mixed with ethyl acetate, centrifuged and the upper layer was evaporated under a nitrogen stream and suspended in acetonitrile for further analysis. Chromatographic separation of epinephrine was done on a Waters ACQUITY UPLC HHS C18 column (2.1× 100 mm, 1.8 µm), and maintained at 40° C. and at a flow rate of 300 µL/min. LC-MS/MS was performed using positive ESIwith a multiple reaction monitoring (MRM) mode (transition: 883.3>170.2) on a triple quadrupole Waters Xevo TQ-S (Waters, Milford, MA) mass spectrometer integrated with a Waters Acquity UPLC controlled by Mass Lynx Software 4.1.

Dofetilide concentration in the media samples was measured after spiking the samples with the internal standard (Dofetilide d4) and separated on a Poroshell 120EC, 2.1×50 mm, 2.7 µm column. Samples were measured using Agilent 6410 triple quad mass spectrometer connected to Agilent 1290 Infinity UHPLC (Agilent Technologies, Santa Clara, CA). MRM transition used was as follows: 442.2>198.0. Doxorubicin and doxorubicinol were assayed simultaneously in samples spiked with internal standard (daunorubicin) using Agilent 6410 LCMS/MS under positive ESI MRM mode (Transitions used: doxorubicin 544.2>398.1; doxorubicinol: 546.2>400.1). All compounds were quantitated by comparing the integrated peak areas of unknown against those of known amounts of purified standards.

### MIRNA Characterization

miRNA was isolated using miRNeasy Mini Kit from (Qiagen, 217004). Samples were shipped on dry ice to Advanced BioMedical Laboratories and assayed using the miRNA 4.0 Genechip Array (ThermoFisher). Results were analyzed using the TAC 4.0 software (ThermoFisher). Additional Ingenuity Pathway Analysis was performed using the methodology described above.

### MIRNA: Gene Set Enrichment Analysis

To assess the activity of miRNAs, gene set enrichment analysis (GSEA) was performed of their targets, as determined by MultiMiR (release 3.11) and RBio-mirGS (update 0.2.12) biocoductor packages (in R) for genes that were differentially expressed after Dox treatment. Since miRNAs generally repress their targets, the sign of the normalized enrichment score was reversed to correctly report their change in activity. For instance, a positive target enrichment for miR-1273a’s targets was reported as a negative NES (NES = -21.31) because it is consistent with its downregulation. Since the analytical form of the null distribution for the NES statistic is not known, a p-value was computed by using an empirical null distribution, generated by random gene sampling. GSEA was performed using fgsea (v1.13.5) package in R.

### Statistics

Data were analyzed in Excel (Microsoft) and graphed in Prism (GraphPad). Data are shown as mean ± standard deviation, for a given number of biological replicates. Significant differences were defined by P<0.05 for all statistical methods, unless otherwise noted. Differences between the experimental groups were analyzed by one-way or multi-way ANOVA. Post hoc pairwise analysis was done using Tukey’s HSD test.

In another aspect of the disclosed subject matter, we established a metastatic niche with interstitial flow, oxygen gradients, and regulatory factors that emulated drug resistance of metastatic cells. We have also bioengineered primary human tumors. Bone sarcoma was formed within bioengineered human bone, subjected to mechanical stimuli and used to link RUNX2 expression to poor survival of sarcoma patients. The bioengineered vascularized neuroblastoma functionally recapitulated vasculogenic mimicry and drug resistance. Bioengineered tumors recapitulated the size and cargo of tumor EVs found in patients, and the tumor mRNA (e.g., EZH2 mRNA) was transferred into the surrounding tissue cells. C ritically, we have shown that, once the matched metastatic and primary tissue is available, we can use network-based methodologies- such as the VIPER algorithm to identify the master regulator proteins that are mechanistically responsible for progression and the FDA approved and investigational compounds that can effectively invert their activity. We have also shown that these analyses can be conducted in single cells, without loss of reproducibility, using meta VIPER, an algorithm that virtually eliminates gene dropout effects associated with low profiling depth.

In another aspect, the system is used for modeling targeted metastasis of breast carcinoma (BRCA). In this regard, the system can initially contain circulating tumor cells, followed by a 3D primary tumor model with patient-derived organoids, all of which can be functionally connected to metastatic target sites (lung, liver, bone; heart as a negative control), all derived from the cells of the same patient, by vascular flow while separated with endothelial barriers. This method can be used to support identification of master regulators of metastatic progression and the drugs specifically targeting metastatic progression, by capturing and analyzing single cells in the primary, vascular, and target organ sites.

In one embodiment, the system comprises: bioengineered metastatic target tissues (lung, bone, liver; heart as negative control) and a 3D breast tumor model based on patient-derived organoids. Patient-specific modeling of metastatic progression via quasi-physiologic integration of tumors and healthy tissues cultured in organotypic conditions by vascular flow can be achieved. We refer to this as “cancer patient on a chip”.

In some embodiments, direct comparative analysis of bioengineered tumors and surgical specimens by single-cell RNAseq in longitudinal studies is performed to establish rigorous methodologies for assessment/validation of biological fidelity. The method can be used for a period of time. For example, long term studies (4-12 weeks) that integrate bioengineering and systems biology approaches to elucidate the mechanistic basis of metastatic progression and drug sensitivity/resistance at the single cell level. Regulatory network based inference and experimental validation of cell-type-specific drug sensitivity in primary and metastatic tumors within a bioengineered tumor context can be performed, if desired.

The availability of predictive in vitro models of human tumors designed to accurately recapitulate key aspects of human pathophysiology is transformative to cancer research and pre-clinical validation of new therapeutic modalities. A tumor can be physiologically integrated with their cognate metastatic sites (lung, liver, bone) via vascular perfusion containing circulating cells. The tumor compartment can be established directly from surgical specimens grown in 3D, organotypic conditions while target metastatic sites and vasculature can be established from blood-derived, patient-matched iPS cells.

By engineering in vitro the patient-specific tumors and host tissues to which they preferentially metastasize, and by physiologically connecting these tissues by vascular flow, we can dramatically improve the ability to investigate mechanisms presiding over metastatic progression in a context bioengineered to recapitulate key aspects of human tumor pathophysiology. The model can provide a biologically meaningful and tightly controllable environment to validate mechanistic drivers and therapeutic predictions of human tumor pathophysiology. The model can provide a biologically meaningful and tightly controllable environment to elucidate mechanistic drivers and therapeutic predictions using bioengineered tumors validated against matched native tumor samples. Our approach is to bioengineer and characterize the native physiologic milieu relevant to the metastatic progression of a patient specific breast carcinoma (BRCA). We can bioengineer host tissues relevant to metastatic progression of BRCA, using patient-matched tumor cells and blood-derived iPS cells. Our focus is on bioengineering BRCA of two molecular subtypes: (a) hormone receptor positive (ER+/PR+) tumors, which prevalently metastasize to bone, liver and lung, and (b) triple-negative breast cancer (TNBC), which prevalently metastasize to lung (low rate to liver and bone).

Tumors can be physiologically integrated with their potential cognate metastatic sites (lung, liver, bone) via vascular perfusion. The tumor compartment can be established directly from surgical specimens grown in 3D, organotypic conditions while target metastatic sites and vasculature can be established from blood-derived, patient-matched iPS cells, under an active institutional review board protocol. The system is imaging compatible and supports long- term culture (4-12 weeks). Biological fidelity and heterogeneity of primary and metastatic sites, as implemented in the context of such vascularized multi-tissue platform, can be validated by single-cell analyses vs. the corresponding native tumor. For these studies, we can recruit a cohort of patients with metastatic tumors. Our ultimate goal is to demonstrate utility of the platform in elucidating mechanisms of tumor progression and drug resistance, by testing drug panels predicted by a novel RNA-seq-based, NY CLIA certified methodology (OncoTreat). Our system can recapitulate key properties of human tumors and enable identification of target proteins that mechanistically drive tumor progression and drug sensitivity/resistance. Thus, the system coupled with the method of use can have broad utility in cancer research and in patient-specific testing of new therapeutic modalities.

In the “cancer patient on a chip” model the system includes vascular perfusion that physiologically integrates circulating tumor cells/bioengineered human tumors with their canonical target tissues to which they preferentially metastasize (lung, liver, bone), all derived from same-patient cells. An exemplary embodiment is a model of breast cancer metastasis, particularly the hormone positive (HR+) and triple negative (TN) invasive ductal carcinomas (IDC), as they are the most common aggressive cancers in women, which lack effective therapeutic modalities. Our 3D BRCA model derived from patient-specific tumor organoids focuses on two molecular subtypes: HR+(ER+/PR+) tumors that prevalently metastasize to bone, liver and lung and triple-negative breast cancer (TNBC), which prevalently metastasize to lung. Samples from chemo-naïve patients presenting with metastatic tumors at diagnosis can be used, resulting in a validated patient-specific model of primary and metastatic breast carcinoma, enabling new insights into patient and tumor-specific drug sensitivity, and providing a platform for cancer research and precision medicine.

In one embodiment, human tumors (osteosarcoma and breast carcinoma)—grown in 3D organotypic conditions—are linked by vascular flow to their canonical metastatic sites (lung, liver, bone). The entire platform can be derived from individual patient cells (both tumor related and normal), support cultures up to 12 weeks, and can be both tunable and imaging-compatible. The platform can be used to study critical dependencies and drug sensitivity of cells representing all steps of metastatic progression. We believe our system can effectively recapitulate critical properties of human tumors and enable assessment of target proteins involved in drug sensitivity/resistance.

Recapitulation of critically relevant pathophysiological parameters of individual tumors include: (i) preservation of genetic, epigenetic, and transcriptional heterogeneity at the single cell level; (ii) minimization of time-dependent genomic and transcriptomic drift for up to 12 weeks (iii) recapitulation of macroscopic parameters, such as hypoxia, vascularization, and stromal representation, and (iv) consistency of drug sensitivity prediction in primary and engineered samples. In one embodiment, we focused on the breast carcinoma, which is characterized by substantial inter-tumor variability, thus testing the platform’s ability to address these goals over multiple genetically and epigenetically distinct backgrounds in female patients.

Method of use 1: Bioengineering patient-specific human tumors and host tissues. Using native scaffolds and bioreactors, we can engineer two primary tumors: osteosarcoma and breast carcinoma. Fluorescently labeled tumor cells and tumor slices (for authentication) can be derived from patient biopsies. Blood-derived iPS cells from the same patient can be used to engineer three normal tissues representing the most frequent metastatic sites for these tumors: bone, lung, and liver. Heart tissue can be used as a negative control for tissue specificity. All tissues can be 1 mm thick, providing a 3D architecture for cells and internal vasculature, while allowing imaging. We can demonstrate that tumor and target tissue cultures recapitulating critical properties and heterogeneity of their human counterparts can be maintained for 4 weeks (routinely) and up to 12 weeks (as needed). Engineered tumors and target metastatic tissues can be characterized/authenticated by exome profiling and single-cell RNA-seq and responses to drugs.

Method of use 2: Establishing a human model of metastasis in an integrated multi-tissue platform. We provide a multi-tissue platform for modeling non-cell-autonomous human pathologies, with all tissues derived from same-patient iPS cells and connected by vascular perfusion.

To provide each tissue with its physiological niche, individual tissue compartments are independently regulated and separated from the vascular perfusion compartment by an endothelial barrier. We can adapt this platform to physiologically integrate primary human tumors (osteosarcoma and breast carcinoma), their canonical metastatic sites (lung, liver, bone), and negative controls (heart). The system can be used to investigate the tissue specificity of vascular endothelium and modulate its permeability by optogenetic methods to investigate its role in each metastatic progression step. A model of metastatic progression of BC cell lines (TNBC, HR+) introduced into circulation under flow and exposed to target host tissues separated by endothelialized barriers within our established multi-tissue platform can be established; (2) we can recapitulate the targeted metastasis previously demonstrated in a mouse xenotransplant model, using cell lines that selectively home to bone or lung; (3)iInvestigate the intravasation potential of patient-derived BC cells from our 3D breast tumor model with patient-derived organoids, metastatic extravasation into host tissues using the methods established by BC cell lines in our platform and the role of patient diversity in metastatic progression via generation of patient-matched, iPSC-derived target tissues. Two metastasis models can be established: (i) circulating tumor cell (CTCs) homing into interconnected host tissues, and (ii) intravasation of tumor cells from the bioengineered primary into the circulation followed by extravasation into host tissues. For both tumors, we can assess whether their preferential metastatic sites are recapitulated in the bioengineered platform, study patient specific metastatic progression over up to 12 weeks, and track the migration of aggressive cells across all three compartments (i.e., primary, CTCs, and metastases).

To model metastasis, we can use our platform that integrates metastatic target tissues (liver, bone and lung; heart as a negative control) via vascular flow mediated by an endothelial barrier. Host tissues can be plugged into the platform, in a desired combination and order, and physicochemical parameters can be optimized to mimic metastatic progression. The heart module can be included both (α) as a negative control for nonselective cell migration, and (b) to extend the platform capability as a cardiotoxic side effect screening device, a major complication of chemotherapy. Initially, TNBC (MDA-MB231) and HR+/HER2-(MDA-MB175, MCF7) cell lines can be used to establish a proof of principle for targeted metastasis for each subtype of breast tumor. Then, lung and bone targeting TNBC cells lines (MDA-MB231-LM, MDA-MB231-BoM can be used to validate the in vitro multi-tissue platform for its ability to recapitulate in vivo phenomena. This can be followed by the introduction of patient specific iPSC-derived host tissues and patient-derived organoid-grown metastatic BC cells to elucidate patient specific effects on intravasation, extravasation, metastatic site BC development and potential drug resistance.

Method of use 3: Elucidating master regulators and key dependencies of metastatic cells in all compartments and predicting/validating their drug sensitivity using the “cancer patient on a chip” model. We can validate/authenticate bioengineered primary tumors and tumor metastases in the “cancer-patient-on-a-chip” platform by directly comparing single-cell RNA-seq and exome profile data for engineered tumors and matched patient biopsies. We can first identify and experimentally validate cryptic tumor dependencies implemented by Master Regulator (MR) proteins responsible for implementing and maintaining the transcriptional state of cancer cells representing different stages of progression (primary, CTCs, and metastases). We can then study unique drug sensitivities/resistance of the same cells—by targeting MR dependencies—to develop new translationally relevant therapies for metastasis, a key challenge in cancer research. This can be accomplished by analyzing single-cell RNA sequencing data with established, NY CLIA certified regulatory network algorithms. Patient-specific drugs identified by these analyses can be experimentally validated on the patient-matched platform.

These methods include: (1) Performing Master Regulator (MR) analysis using scRNASeq signatures of engineered primary tumors, vascular flow CTCs, and tumor metastases in host tissues, to identify (i) MR proteins that mechanistically determine tumor cell priming for metastatic progression and (ii) unique CTC and metastatic cell dependencies. (2) Performing OncoTreat analysis, using the same scRNASeq signatures, to identify small molecule inhibitors capable of reversing the activity of subpopulation-specific MR proteins to induce cell demise or reprogramming to a non-invasive state. (3) Experimental validation of these findings in the “cancer patient on a chip.”

We have pioneered the use of lineage-specific regulatory networks, inferred de novo from large-scale molecular profile data, for the identification of MR proteins that mechanistically implement (i.e., via the transcriptional targets they regulate) cell state transitions. These methodologies, integrated into the VIPER algorithm, were successful in elucidating novel mechanisms of tumorigenesis and drug sensitivity in glioma, leukemia, lymphoma, prostate, neuroblastoma, neuroendocrine tumors, and breast cancer, among others. Relevant to this proposal, VIPER identified MR proteins controlling metastatic progression of ER+ and TNBC breast carcinoma, whose inhibition in vivo caused 100 to 1,000-fold reduction in lung metastasis burden. By coupling VIPER analysis with large-scale RNASeq profiles of tumor cells treated with ~400 compounds (FDA-approved or phase ⅔ clinical trials), we were able to identify drugs that revert the activity of MR proteins, on an individual tumor basis, thus inducing tumor state collapse and loss of tumor viability. The corresponding algorithm (OncoTreat), which was CLIA certified by NY State Dept. of Health and recently tested in an N-of-1 study at Columbia (IRB-AAAN7562). Out of 39 inferred drugs that were evaluated in PDX models from patients with 6 different aggressive/metastatic malignancies and failed 3 to 6 lines of therapy, 23 induced stable disease or partial remission, vs. none of the 28 drugs in the negative control arm (p<10⁻¹²⁸). Overall, these methodologies have yielded six clinical studies. The PLATESeq RNASeq technology developed jointly by the Sims and Califano labs was used to generate comprehensive RNASeq profiles of drug perturbations for >10 tumors including breast cancer. The meta VIPER algorithm, designed for scRNASeq analysis, supports MR and OncoTreat analysis from single cell profiles with no loss of sensitivity and increased specificity compared to bulk tumors. Indeed, we showed that drugs predicted at the single cell level by OncoTreat successfully depleted the predicted subpopulations in patient derived GBM explants.

To assess clonal heterogeneity, we can systematically authenticate tumor tissues (primary and metastatic) cultured in the bioengineered platform, at the single cell level. Specifically, we can compare gene expression and MR-activity profiles of single cells and bulk exomes from bioengineered tumors and surgical specimens, across three compartments: (a) primary tumors (Method of Use 1), (b) CTCs - infused or intravasated from the primary tumor compartment, and (c) bone, lung and liver metastases (Method of Use 2). Due to metaVIPER’s ability to effectively reduce batch effects and other technical artifacts, these analyses can help identify cross-compartment subpopulations of related cells, by MR overlap analysis. For instance, we can use meta VIPER to identify primary tumor cells that most closely match to CTCs and metastases, assess potential priming for metastatic progression, and validate by lineage-tracking. In addition, cells from all three compartments can be analyzed by OncoTreat to identify drugs and drug combinations most likely to invert MR protein activity, thus inducing cell demise or reprogramming to a non-invasive state (Method of Use 3).

Predictions can be tested in the engineered platform over 4-12 weeks of culture, by assessing drug-mediated depletion of cellular compartments and overall reduction of metastatic cells in target tissues. We can thus be able to isolate and study the individual processes that comprise metastatic progression, identify critical cell subpopulations manifesting sensitivity to orthogonal treatments, and test panels of drugs predicted by scRNASeq. We envision offering this platform for broad use in studies of human cancer, and demonstrating its utility, predictability and reproducibility.

In one embodiment, bioengineering of osteosarcoma and breast cancer using patient’s tumor cells and a native-like matrix is provided. The method can include patient-specific platforms to study metastatic progression to canonical target tissues (lung, bone and liver; heart as a negative control), with patient-matched, iPS-derived vasculature and target tissues, and matched extracellular matrix and regulatory signals; Patient-specific modeling of metastatic progression via quasi-physiologic integration of tumors and healthy tissues cultured in organotypic conditions by vascular flow - the “cancer patient on a chip” platform; Establishment of a physiological endothelial barrier between tissue compartments and vascular flow, whose vascular permeability can be modulated via optogenetic methods; Direct comparative analysis of bioengineered tumors and surgical specimens by single-cell RNA-seq, in longitudinal studies, to establish rigorous methodologies for assessment/validation of biological fidelity; Long term (4 weeks) studies integrating bioengineering and systems biology approaches to elucidate the mechanistic basis of metastatic progression and drug sensitivity/resistance at the single cell level; Regulatory-network-based inference and experimental validation of cell-type-specific drug sensitivity in primary and metastatic tumors in a bioengineered tumor context. In vitro tumor platform for drug testing.

The bioreactor system, for example 100, 200, 300 described herein, can be used to enable modeling of metastatic progression, comprising a complex sequence of dynamic events that are difficult to recapitulate in existing models and surgical specimens. Tumor cells invade and disseminate via the vasculature, colonizing perivascular tissue niches around the capillaries in tumor-specific target tissues. Vascular flow brings the gradients of shear, oxygen, nutrients and signaling factors into the tumor tissue, thereby maintaining the tumor niche, while the extracellular matrix (ECM), endothelial cells (EC), stromal and immune cells regulate intravasation, extravasation and metastatic homing.

We validate the system utility in studies of two types of highly metastatic tumors: breast carcinoma (BRCA) and osteosarcoma (OS), in “organs on a chip” platforms. The platform can contain a bioengineered human tumor and metastatic target sites (lung, liver, bone; heart as a negative control), all derived from the cells of the same patient, and functionally connected by vascular flow. The studies can be designed to support identification of master regulators of metastatic progression and the drugs specifically targeting cells during metastatic progression, by capturing and analyzing single cells in the primary, vascular, and target organ sites. We can validate metastatic progression models over a period of 4-12 weeks, by directly comparing gene expression and VIPER-inferred protein activity profiles of cells in matching native and bioengineered tumors at various time points.

By engineering in vitro both the patient-specific tumors and host tissues to which they preferentially metastasize, and by physiologically connecting these tissues by vascular flow, we can dramatically improve our ability to investigate multiple mechanisms presiding over metastatic progression in a physiologically-relevant human tumor context. These models provide a meaningful and highly controllable environment to validate mechanistic drivers and therapeutic predictions derived from bioengineered and patient-matched tumor samples. We can bioengineer host tissues relevant to metastatic progression of breast carcinoma and osteosarcoma, using patient-matched tumor and iPS cells. Generally, tumor and host tissues are engineered, then the engineered tumors can be validated by comparison to the patient samples. We can integrate the tumor and host tissues by vascular flow and investigate tissue-specific intra/extravasation of track-labeled tumor cells, under tight EC barrier control in the system described herein. Single-cell RNA seq of native and bioengineered tumors can be used to infer mechanistic determinants of metastasis and drug sensitivity in “cancer patient on a chip” model.

All data including outlier values can be documented. The criteria to accept data can be stated. Blind measurements and quality control metrics can be used systematically to ensure unbiased study designs, data collection and interpretation. Furthermore, use of patient-matched iPS and tumor cells can enable rigorous studies of metastatic progression in an isogenic background, with each patient treated as an independent study (N = 1).

Consideration of relevant biological variables. Rather than addressing the broad diversity of tumor progression and drug sensitivity mechanisms in a diverse population - an impossible task given the relatively small cohort size, our goal can be to demonstrate the proposed platform’s ability to faithfully recapitulate critically relevant pathophysiological parameters of individual tumors. These include, among others, (i) preservation of genetic, epigenetic, and transcriptional heterogeneity at the single cell level; (ii) minimization of time-dependent genomic and transcriptomic drift for up to 12 weeks (iii) recapitulation of macroscopic parameters, such as hypoxia, vascularization, and stromal representation, and (iv) consistency of drug sensitivity prediction in primary and engineered samples. With this in mind, we focused on two tumors characterized by substantial inter-tumor variability, thus supporting the platform’s ability to address these goals over multiple genetically and epigenetically distinct backgrounds with >50% representation by female patients.

In one embodiment the method includes (1) Bioengineering breast adenocarcinomas (BRCA) and osteosarcomas (OS) from primary tumor cells with infiltrating stromal/immune compartments established from patient-matched iPSCs. (2) Validating the fidelity of engineered vs. patient tumors. (3) Bioengineering and validating metastatic host tissue models, including lung and liver (for BRCA and OS), bone (for OS) and heart (as a negative control).

The tumor microenvironment plays a critical role in controlling tumor initiation, progression, and drug resistance, via mechanisms ranging from angiogenesis and reprograming to immunoevasion. This understanding has been seminal in fostering convergence of cancer biology and tissue engineering, towards developing more faithful and physiologically relevant models of human cancer, where the tumor compartment co-exists with stroma and vasculature. Yet, faithful modeling of complex multi-organ processes, underlying metastatic progression, is beyond the capabilities of tumor spheroids and similar systems. There is a critical need to better understand the key drivers of metastatic progression and the pharmacologically accessible dependencies of metastatic tumors. By co-engineering patient-specific tumor and normal tissues, the platform can provide a quasi-physiologic environment for lineage-tracking of aggressive cell subpopulations and allow identification of novel, pharmacologically actionable drug targets and therapies.

Collection of tumor and blood samples. Operable tumors, ≥1.5 cm³ in size, can be collected from 20 patients diagnosed with metastatic disease under our active IRB protocols (IRB-AAAN7562, AAAB2667) and fully characterized by high-depth (120X) exome and RNASeq profiling. Both the primary site and one or more metastatic sites can be biopsied. 1-mm tumor slices can be sectioned using a Vibratome (Molecular Pathology Shared Resource, Herbert Irving Comprehensive Cancer Center) and cultured in our platform as an organotypic benchmark for validation of bioengineered tumors. The tumor mass can be dissociated into single cells, labeled for cell tracking (bioluminescence, fluorescence), and used to bioengineer tumors. Dissociated cells can be analyzed by single-cell RNA-seq (scRNASeq) for later comparison with bioengineered tumors. We can also collect blood samples (8 mL) from the same patients, to (a) characterize the patient-specific secretome using our established methods, and (b) generate patient-specific iPS cells. Tumor-associated exosomes from the patient’s blood can be compared to those secreted by bioengineered tumors for fidelity assessment.

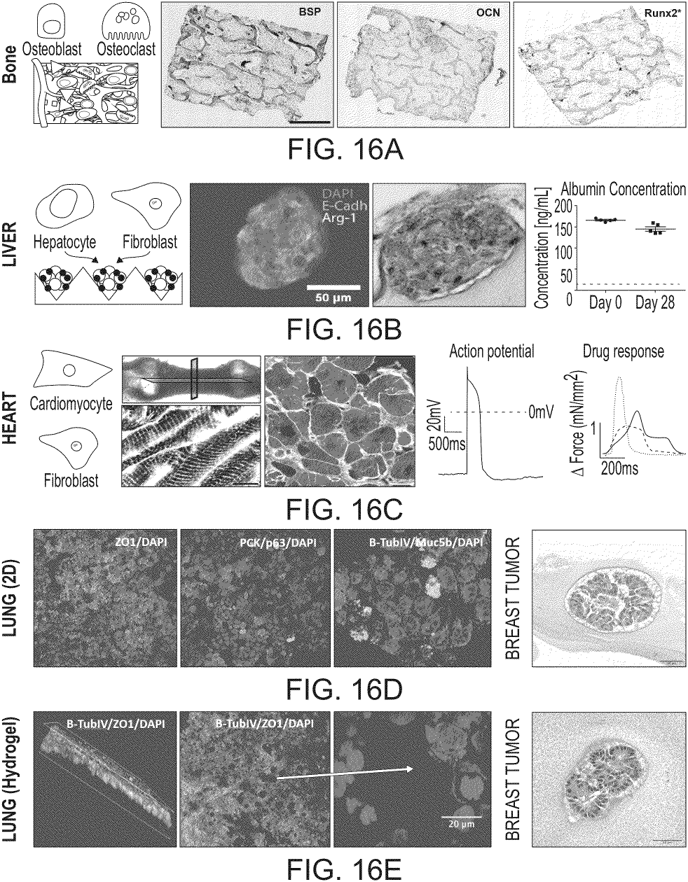

FIGS. 16A-E show aspects of bioengineering of matured canonic target tissues for breast cancer metastasis (bone, liver, lung), heart tissue (negative control for metastasis), and breast cancer organoids. All tissues are derived from a single batch of iPS cells. Tumor organoids are derived from matching tumor samples from the same patient.

Bone is the most common site for metastatic relapse of breast tumors, particularly of the HR+ subtypes and tends to be the first site of metastasis in a significant proportion of patients [22-24]. Bone tissue can be formed from iPS-derived MSCs (giving rise to osteoblasts) and monocytes (giving rise to osteoclasts), in perfused bone scaffolds, to display the physiologic osteolytic cycle and active remodeling. (FIG. 16A).

Liver tissue, is the second frequent metastatic site for HR+ BRCA whereas liver metastases are less common for TNBC but are associated with poor prognosis. Engineered liver tissues can be formed by aggregating hepatocytes and fibroblasts (iPS-derived) into phenotypically stabilized hepatic units. Hepatic aggregates are then encapsulated in decellularized liver matrix to form the engineered liver tissues and matured in culture. Liver tissues displayed consistent production of urea (FIG. 16B) and albumin, and strong activity of liver enzymes for drug metabolism, resulting in physiologic profiles of drug safety and efficacy.

Heart muscle is formed from iPS-derived cardiomyocytes and fibroblasts in hydrogel around flexible pillars, and exposed to an electrical pacing regime optimized to induce tissue maturation. After only 4 weeks of culture, these tissues achieved adult-like gene expression, ultrastructure with networks of t-tubules, oxidative metabolism, positive force-frequency relationship, and functional Ca handling (FIG. 16C).

Lung tissue—the main metastatic site for TNBC—can be formed using decellularized lung matrix with fully preserved matrix architecture and composition by following two different methods established in our lab. Human bronchial epithelial cells (BEAS2B line, primary hBECs or iPS-derived lung progenitors can be either seeded on lung ECM-coated transwells of our multi-tissue platform (lung 2D model) or embedded in lung ECM hydrogels. The cells can be cultured as submerged in medium for a proliferation phase of 3-5 days followed by air-liquid interface (ALI) culturing up to 4 weeks. In both models, bronchial epithelial cells show a fully differentiated phenotype after 4 weeks of ALI culture. Immunofluorescence staining revealed b-tubulinIV positive ciliated cells, Muc5b positive goblet cells and p63 positive basal cells (FIG. 16D). Epithelial markers E-cadherin and pan-cytokeratin as well as tight junction formation indicated by ZO-1 staining were observed in our airway models. Interestingly, the lung hydrogel model revealed formation of numerous airways in the form of holes decorated with ciliated cells (FIG. 16E).

Our lab has established several strategies for lung decellularization, through the use of human and porcine lungs. Our 3-[(3-Cholamidopropyl) dimethylammonio]-1-propanesulfonate (CHAPS)-based decellularization protocol was optimized to fully preserve the lung matrix architecture and composition (FIG. 16D). We have also developed methods for targeted removal of lung epithelium while preserving intact pulmonary vasculature and interstitium. These methods can allow customizing our engineered lung models to increase complexity and inclusion of multiple ECM components and cell types. Our decellularized lung matrix-based hydrogels provide a tunable microenvironment, ideally suited for cell-matrix interaction studies. We established alginate-reinforced, decellularized lung hydrogels that allow double-crosslinking (thermal and calcium-mediated) and manipulation of ECM mechanics via cation content, thus supporting both physiological and pathological ranges of lung ECM stiffness. Lung tissue stiffening may occur due to increased age or pathological conditions such as idiopathic pulmonary fibrosis (IPF), previously associated with inflammatory conditions and chemo-resistance. We can study the effect of increased lung tissue stiffness on pulmonary cell phenotypes, gene expression, proteome and secretome (miRNAs, EVs) and the related effects of these factors on metastatic TNBC cell homing to the lung.

Osteosarcoma Model: Tissue-engineered osteosarcoma models (TE-OS) were successfully established by culturing osteosarcoma cell lines in a bioengineered human bone generated by differentiation of human iPSC-derived mesenchymal stromal cells into osteoblasts and human iPSC-derived monocytes into osteoclasts. Osteosarcoma cell aggregates were infused into the bone niche, cultured for 2 weeks, and shown to recapitulate key features of their human counterpart. These tumors displayed marker proteins of differentiated osteoblasts and osteoclasts, including osteopontin (OPN), bone scaffold protein (BSP), osteocalcin (OCN), and tartrate-resistant acid phosphatase (TRAP). Staining for alkaline phosphatase and von Kossa indicated bone differentiation. Quantitative PCR (qPCR) detected markedly elevated gene expression, compared to cell monolayers, for markers of osteoblasts and osteoclasts. Fluorescent pimonidazole staining identified a hypoxic core, an important feature of OS microenvironment.

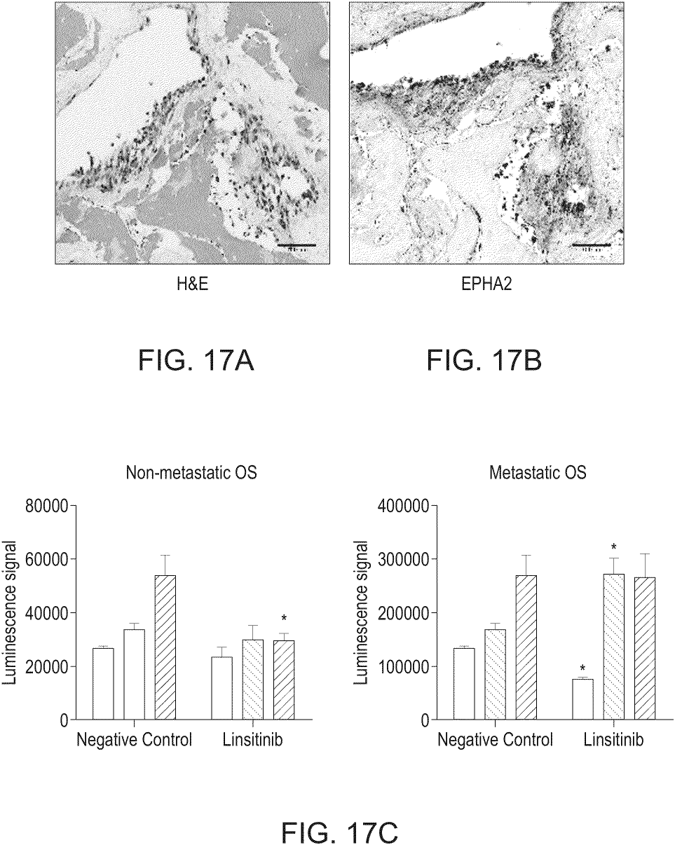

To vascularize TE-OS, it is critical to induce vascular development prior to osteogenesis (FIGS. 17A-C), a paradigm that recapitulates native bone vascularization. The TE-OS contained OS surface markers (EPHA2, CD133), and re- expressed genes related to focal adhesion and cancer pathways (qPCR). Recapitulation of hypoxia, angiogenic potential, and vasculogenic mimicry were also observed, and associated with increased expression levels of HIF-1, VEGF and endothelium-associated genes (LAMC2, TFPI1), respectively. Bioengineered human bone displayed BSP loss, increased trabecular spaces, and slightly decreased bone volume density, all of which are hallmarks of early lytic lesion formation in patients. TE-OS can comprise a native bone ECM- derived scaffold, iPS-derived osteoblasts and osteoclasts, and patient-matched OS tumor cells. Extreme OS heterogeneity makes it an ideal tumor for individualized modeling. OS cells can be isolated from both primary and secondary (metastatic) sites, with less than 90% necrosis, from chemo-naive patients metastatic at diagnosis. Analysis of morphology, doubling time, migration rate (wound healing assay) and proteins associated with osteosarcoma progression (p53, phosphorylated SRC kinase) can be performed. Cells can be lentivirally transduced with GFP-luciferase using established protocols, to track them in culture.

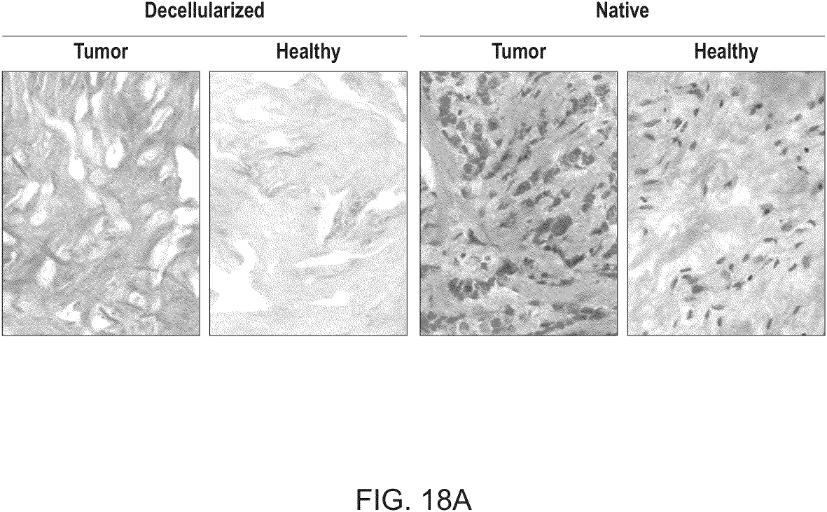

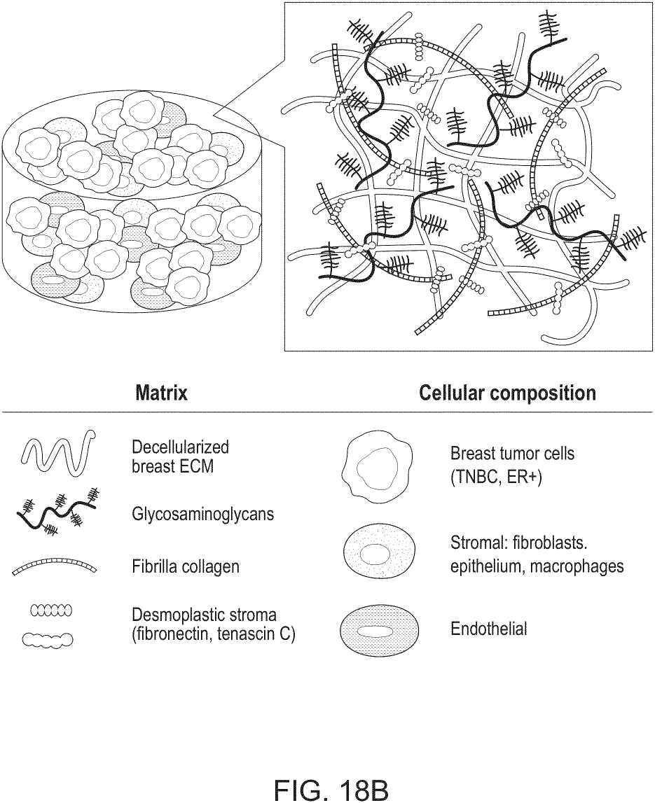

Breast carcinoma model: Native ECM can be processed into tunable scaffolds and used as a niche for patient-derived tumor cells retrieved from biopsies of primary and matched metastatic sites of chemo-naive patients (metastatic at diagnosis), and supporting cells (FIGS. 18).

FIGS. 18A-B show aspects of bioengineered breast tumor tissue. FIG. 18A shows H & E staining of native (top) and decellularized (bottom) healthy and tumorous breast tissue sections showing preservation of tissue architecture. FIG. 18A shows a schematic of the proposed breast tumor model with controlled biochemical, mechanical and cellular manipulation and complexity.

We can focus on two molecular subtypes: estrogen receptor positive (ER+) tumors, which prevalently metastasize to bone, and triple-negative breast cancer (TNBC), which prevalently metastasize to lung and liver. Fibroglandular mammary tissue can be extracted following surgical excision, to derive breast tissue ECM for the engineered tumor matrix. Our lab has established protocols for the production of ECM hydrogels and scaffolds that span 24 organs including lung, liver, heart, bone, and breast tissue. To provide a spectrum tumor-like matrix characteristics, we can integrate the native ECM within interpenetrating networks that can support independent modulation of stiffness, composition, and dynamics (FIGS. 18). Breast cancer is marked by significant changes in ECM composition and mechanics, including stiffening of interstitial ECM by fibrillar collagens (I, III, V), fibronectin, hyaluronan, versican and syndecan. In our model, fibrillar collagen I can be incorporated into breast tissue hydrogels via thermal gelation, while hydrogel stiffness can be modulated via enzymatic crosslinking in the presence of lysyl oxidase (LOX) or phenol oxidases (tyrosinase, laccase). To mimic the tumor desmoplastic stroma, glycoproteins fibronectin and tenascin C can also be incorporated. To assess aberrant increase in ECM proteoglycans, we can modify hyaluronan, heparin and chondroitin sulfate with tyramine groups that can allow incorporation into the native ECM via tyramines coupling with ECM polymers carrying tyrosine residues.

Mechanical properties of tumor and normal-related tissue are distinctly different. Elevating stiffness alone, independent of composition, may be sufficient to induce malignant transformation of mammary epithelial cells. Breast carcinoma can develop from alterations in multiple pathways and manifest a range of mechanical properties that also depend on the spatial scale at which they are probed. For instance, indentation testing of 169 breast tumors, using a 5 mm probe, showed tumor stiffness ranging from 10 kPa - 50 kPa, depending on tumor grade and structural makeup. At the other extreme, atomic force microscopy revealed significant nanoscale-level mechanical heterogeneity within normal breast tissue and malignant lesions. We aim to recapitulate this mechano-complexity by local matrix stiffness modulation. Our ECM hydrogel allows three crosslinking mechanisms: thermal and enzymatic gelation of fibrillar collagen; enzymatic and light-mediated crosslinking of tyramine-modified biopolymers (hyaluronan, heparin, chondroitin sulfate). Oxidation of rosebengal and eosin Y-activated tyramine moieties induced by visible light was shown to enable multi-photon patterning and can allow local changes in ECM stiffness and recapitulation of stiffness heterogeneity.

Vascularization. Primary tumor cells enriched by iPSC-derived supporting fibroblasts, EC, and macrophages, can be encapsulated in ECM hydrogels, and cultured for ≥4 weeks to allow monitoring of key variables: cell growth, DNA and RNA profiles, metabolic activity, cell morphology and cytoskeleton, proliferation (Ki67) and proteins to characterize subtype-specific breast malignancy. Patient-derived EC and primary tumor cells can be co-cultured in the ECM-hydrogel matrix.

Cell-matrix reciprocal interactions. While ECM mechanics and composition can modulate tumor behavior, tumor cells can reciprocally alter their microenvironment. Independent control of ECM parameters can help disentangle the relative contribution of cell-mediated ECM changes from those resulting from modulation of ECM by genetic, environmental, and pathogenic factors. Such cell-matrix feedback loop has been established in several models. Here, we can validate that our model captures the dynamics of interactions that are important for the study of vascular permeability and intra/extravasation.

Validation. Engineered tumors can be validated against native tumors via scRNASeq, mutational and exome profiling, IHC, metabolic, and secretome analyses. Cellular and macroscale changes in bioengineered tumor mechanics can be measured in real time, using optical elastography and compared to the native tumors.

Establishment of patient-specific host tissues: Host tissues can be engineered using iPSCs-derived cells, within a native, biomimetic ECM environment designed to promote phenotypic maturation. For bone - a metastatic site for ER+ breast tumors and osteosarcoma, we can incorporate EC, osteoblasts, and osteoclasts into decellularized bone matrix, thus recapitulating bone formation, maintenance, and regeneration. To engineer liver - a metastatic site for both TNBC and OS, we can combine aggregates of iPSC-derived hepatocytes and fibroblasts (in a 3:2 ratio) into fibrin hydrogel. Such culture supports maturation of liver tissue, and enables sustained production of urea and albumin. We engineer from iPSCs heart tissue with unprecedented level of maturity, displaying: positive force-frequency response, t-tubule networks, oxidative respiration, and physiologic drug responses. In the proposed design, heart tissue can provide a negative control for metastatic progression, ensuring that observed metastases result from tumor-specific homing mechanisms rather than from nonselective cell migration into any tissue.

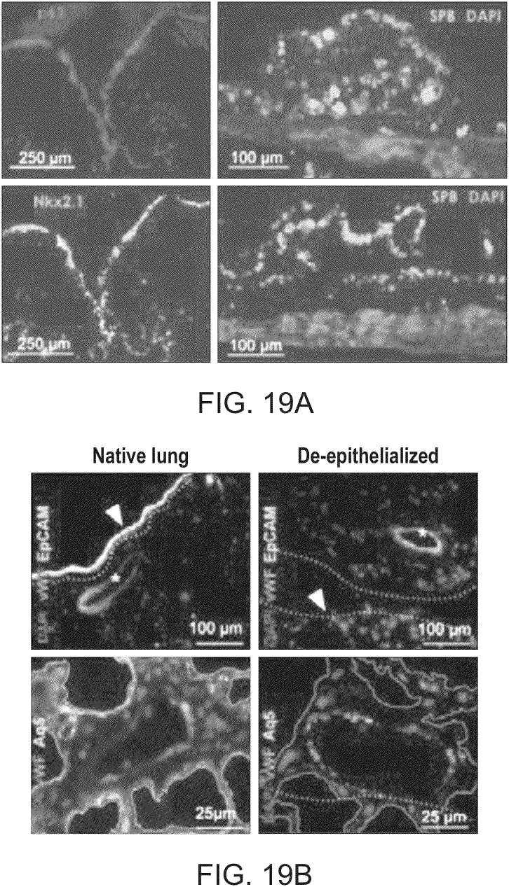

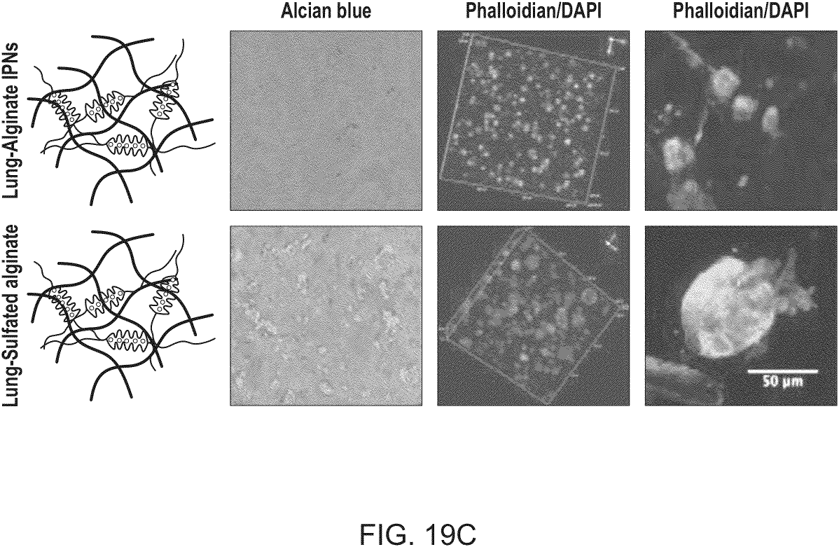

To further increase the proposed platform’s relevance, we can also engineer lung tissue—a metastatic site for both TNBC and OS. 3-[(3-Cholamidopropyl) dimethylammonio]-1-propanesulfonate (CHAPS)-based decellularization protocol fully preserves the lung matrix architecture and composition (FIGS. 19A-C).

FIGS. 19A-C show aspects of bioengineered lung tissue. FIG. 19A shows stem cell-derived lung bud organoids differentiated on lung EMC with co-expression of pulmonary markers p63, Nkx2.1 and SPB. FIG. 19B shows targeted decellularization of lung epithelium while preserving lung vasculature and interstitium. FIG. 19C shows lung ECM-alginate/alginate sulfate networks and encapsulation of bronchial epithelium.

We can apply methods for targeted removal of lung epithelium while preserving intact pulmonary vasculature and interstitium in our engineered lung models and increase complexity through inclusion of customized ECM components and multiple cell types. To enhance biological fidelity of engineered lung tissue, hydrogel-based lung ECM, supports encapsulation of pulmonary cells in a “tunable” microenvironment ideally-suited to cell-matrix interaction studies. Alginate-reinforced, decellularized lung hydrogels allows double-crosslinking (thermal and calcium-mediated) and manipulation of ECM mechanics, thus supporting both physiologic and pathologic-grade lung ECM stiffness (FIGS. 19). Lung tissue stiffening may occur due to increased age or pathological conditions such as idiopathic pulmonary fibrosis (IPF), previously associated with inflammatory conditions and chemo-resistance. We can study how increased stiffness of lung tissue on pulmonary cell phenotypes, gene expression, proteome and secretome (miRNAs, exosomes) and the related effects of these factors on metastatic homing of BRCA and OS cells to the lung.

Another pathological alteration in lung ECM composition is an increase in sulfated glycosaminoglycans, which commonly follows lung inflammation and interstitial pulmonary fibrosis. A biomimetic model of pathological lung tissue formed by interpenetrating lung ECM networks using sulfated glycosaminoglycan-mimetic alginate sulfate, through tunable sulfation and precise control of hydrogel mechanics, enable studies of aberrant GAG content effects on pulmonary cell phenotype and function. Our preliminary data show that such increases in GAG contents of the ECM drive pathological growth of bronchial epithelial cells and lung adenocarcinoma cells with onset of epithelial-mesenchymal transition (FIGS. 19). Therefore, we can use this tissue model to identify parameters that promote metastatic homing of breast tumor and OS cells. Engineered lung tissues can be populated with IPSC-derived lung bud organoids. Pulmonary cell phenotype can be assessed by monitoring cell viability, metabolism, proliferation and expression of lineage markers. Further characterization can be done to investigate the cell secretome from engineered matrices with different mechanical and biochemical properties, to identify potential factors/markers that could serve as homing factors for metastatic tumor cells.

We can use native tumor benchmarks to show that engineered tumors recapitulate key hallmarks of metastatic progression, including expression of proliferative and metastatic markers. We believe breast tumor progression can increase as a function of matrix stiffness, collagen fiber alignment, and angiogenesis. Rigorous characterization of these cells can be performed to understand any aberrant behavior of the bioengineered models due to phenotypic heterogeneity. If extended tumor culture (>4 weeks) poses challenges, we can adjust perfusion conditions to improve long-term control of nutrients, oxygen and regulatory factors.

To establish a model of metastasis in an integrated human multi-tissue platform, the method includes (1) Physiological integration of tumor and metastatic host tissues via vascular flow; (2) Modeling metastatic progression into the circulation and the host tissues, by tracking labeled tumor cells. (3) Investigating the mechanistic role of the endothelial barrier in tumor progression.

To model metastasis, the platform that integrates engineered tumors and metastatic homing tissues (liver, bone and lung; heart as a negative control) via vascular flow mediated by an endothelial barrier. Tumors and host tissues can be plugged into the platform, as described above,, in any desired combination or order and physicochemical parameters can be optimized to mimic metastatic progression. The heart module can be included both (a) as a negative control for nonselective cell migration, and (b) to extend the platform capability as a cardiotoxic side effect screening device, a major complication of chemotherapy.

A stable endothelial barrier such as 105, 205, 305, can be established by placing iPSC-derived ECs onto an elastic membrane and exposing them to physiological flow shear. To physiologically integrate engineered tumors and host tissues by vascular flow, they can be connected in modular format by placement into chambers above the endothelium-like membrane that separates tissue and vascular media, thus enabling vasculature-mediated tissues crosstalk (FIGS. 1). The endothelial barrier can support use of optimal, yet distinct medium and culture conditions for each tissue - an approach akin to compartmentalization in the body that avoids the use of suboptimal “cocktail” media. This approach promotes long term culture (4-12 weeks), with multiple on-line readouts (cell tracking, viability, function,) in longitudinal and dynamic studies.

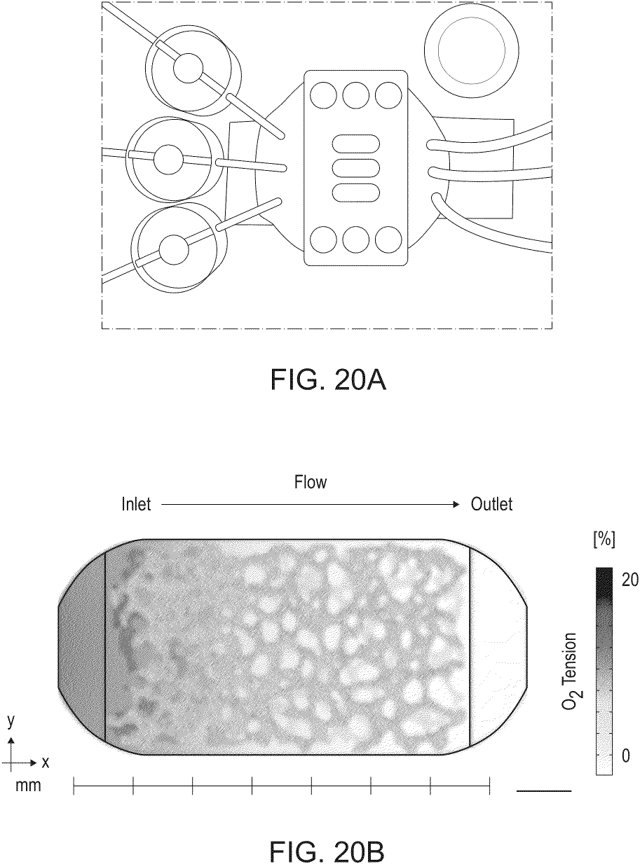

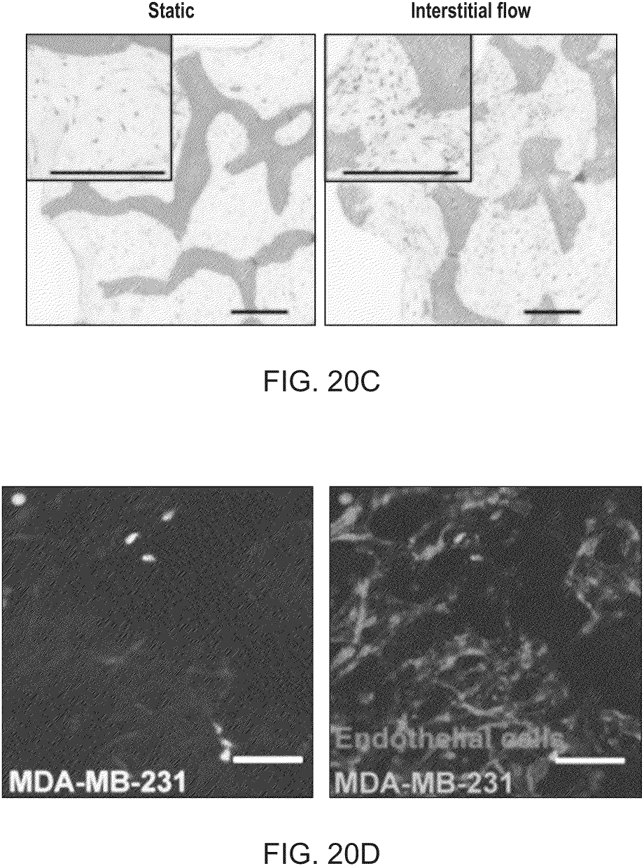

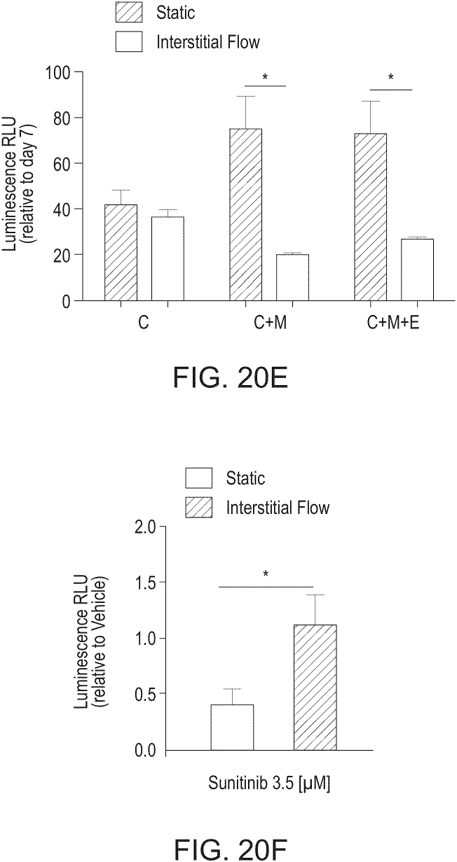

Tracking extravasation and seeding of circulating tumor cells (CTC): We label tumor cells using fluorescent/bioluminescent tags and infuse them into the circulation to study their extravasation through endothelial barrier and seeding into host tissues. In the absence of the endothelial barrier, cells non-selectively colonize all host tissues. We characterize tumor cells and their preferential homing to specific host tissues to assess the model’s ability to mimic physiologically-relevant metastatic progression. A patient-specific platform, with tunable mechanical and biochemical host tissue properties, may support preferential metastasis of TNBC, ER+, and OS cells into lung /liver, bone, and bone/lung compartments, respectively, but not to the heart compartment. We can further manipulate physicochemical properties of host tissues (stiffness, inflammatory fibrosis, aberrant glycosaminoglycan content, to mimic organ conditions of high-risk patients, due to age, or inflammation, and study their effects on CTC efficiency and specificity. We can also control the cellular complexity of engineered host tissues and assess which stromal environments favor metastatic cell homing. We can thus evaluate a variety of stromal cells (tissue-specific endothelium, fibroblasts, and stroma; tissue-resident macrophages). We can characterize the effects of angiogenic factors (VEGF, FGF, EGF) on metastatic cell homing (FIGS. 20A-F).

FIGS. 20A-F show aspects of breast tumor cells into the human bone perivascular niche-on-a-chip. FIG. 20A shows the microfluidic chip with the engineered bone perivascular (BoPV) niche. Interstitial flow and infused tumor cells mimc metastatic colonization. FIG. 20B shows oxygen tension throughout the BoPV niche after 2 weeks of culture. FIG. 20C shows H & E staining of the BoPV niche cultured statically or under interstitial flow showing cell growth and capillary formation. FIG. 20D shows breast cancer colonization under static and low conditions. FIG. 20E shows a luciferase assay used to assess cancel cell growth in the BoPV niche containing only cancer cells (C) or with MSCs (C + M) and endothelial cells (C + M +E) at day 7. FIG. 20F shows cancer cell survival and drug resistance in the BoPV niches after treatment with 3.5 µM sunitinib for 3 days, assessed by luciferase assay.

The cells that invade the host tissues can be characterized by single-cell RNA sequencing (see method of use 3 for details), to determine their phenotypes associated with metastatic seeding into the specific host tissues. The pathology of host tissues colonized with tumor cells can be characterized by histology/IHC-staining, and compared with clinical specimens. We can also conduct secretome analyses for tumor-specific, metastasis- driving extracellular vesicles (EVs) before and after the tumor cells are introduced into the circulation, before and after homing, to understand what signaling events could underlie the metastatic phenomena. The cells that metastasized into the host can provide a reference for tracking of tumor cells during metastasis, where we can investigate tumor cell intravasation into circulation and extravasation into host tissues. To validate the model, we can compare metastasis in the platform with the metastatic bone tumors from patients as benchmarks, and evaluate the tissue specificity and colonization potential of metastatic cells in the platform.

Modeling tumor metastasis into the host tissues.. Primary tumor cells can be labeled (bioluminescence to track cell motion and proliferation; fluorescence to monitor cell colonization and seeding). The tumor model can be connected by vascular flow to the lung, liver, bone and heart tissues. We can assess whether tumor cells (i) migrate out of the engineered tumors, intravasate through the EC barrier and enter circulation, (ii) arrest along the vascular wall of the specific host tissue, (iii) extravasate through the EC barrier and (iv) seed and colonize the host tissue. Important variables can be tumor characteristics, the host tissue type, order of vascular flow through the platform, and endothelial permeability. We can manipulate tumor characteristics as discussed (method of use 1), to enable monitoring of cellular events and testing the effects of engineered tumor ECM and cell types on metastatic potential along the four main steps of metastasis. Similarly, the effects of host tissue properties (ECM stiffness and composition, cellular composition) can be investigated. We can first focus on studies of tumor cell intravasation and metastatic potential as a function of endothelial stability and permeability.

Investigating the mechanistic role of the endothelial barrier in tumor metastasis: Cancer progression and treatment depend on the tumor cell ability to migrate to distant sites, and on drug transport from vascular circulation to these sites. Blood vessels have dynamic and heterogeneous barriers that select which entities can pass through. Since blood vessel permeability is highly variable across organs, tumors, and patients, it is critical to reproduce such heterogeneity in cancer models. By tracking labeled tumor cells and controlling vascular permeability, we can study the mechanistic role of the vascular barrier in metastasis.

Assessing specification and permeability of vascular endothelium: Vascular permeability is regulated by EC lining of the vessels, serving as a gatekeeper for the exchange of cells and molecules between tissue and blood. Tumor cells increase endothelial permeability, suggesting their role in intra/extravasation. While some organs (e.g. heart and lung) have tight endothelium allowing passage of only small molecules (<2 nm), inflamed and tumor-related tissues present large endothelial gaps and high permeability. Vascular permeability also influences the maximum achievable drug concentration in tumors. To be predictive, tumor models must recapitulate the biological complexity and heterogeneity of tissue/blood- vessel interactions. We can investigate specification of endothelium (using IHC for lineage-specific endothelial markers), and changes in permeability (using fluorescently labeled dextrans).

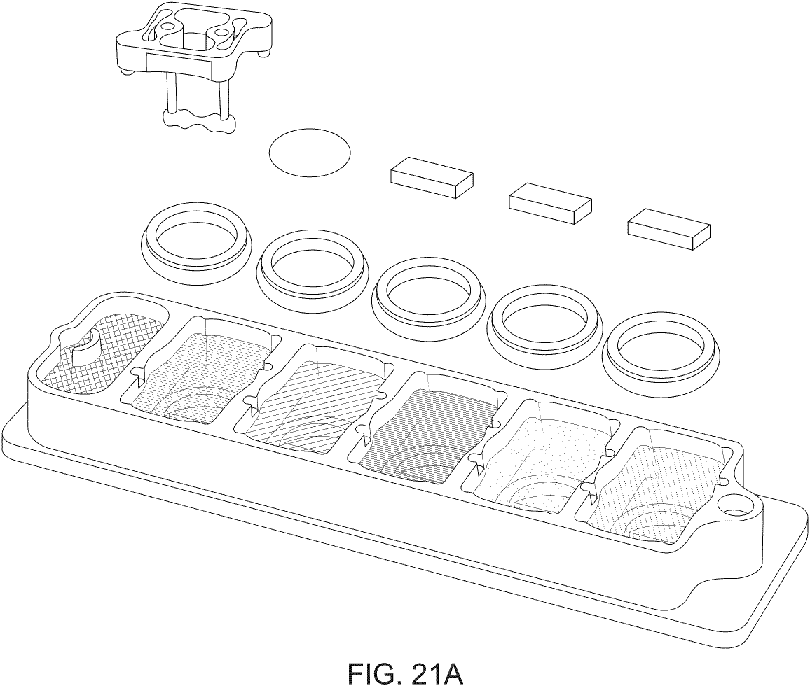

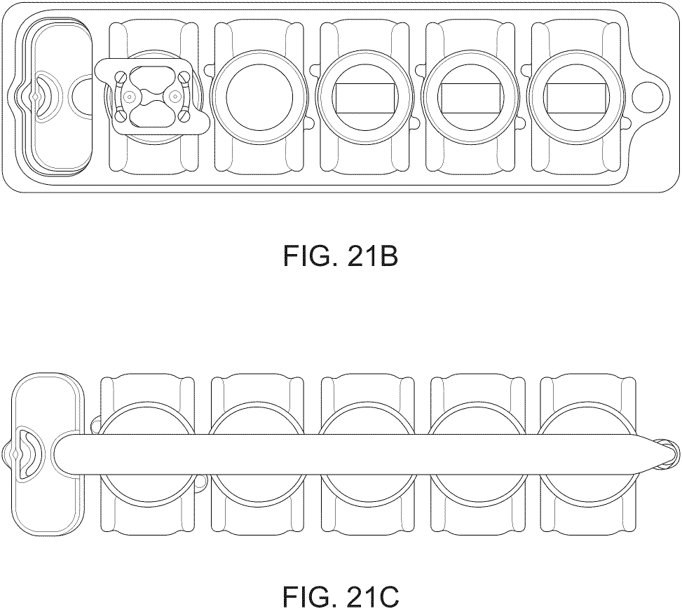

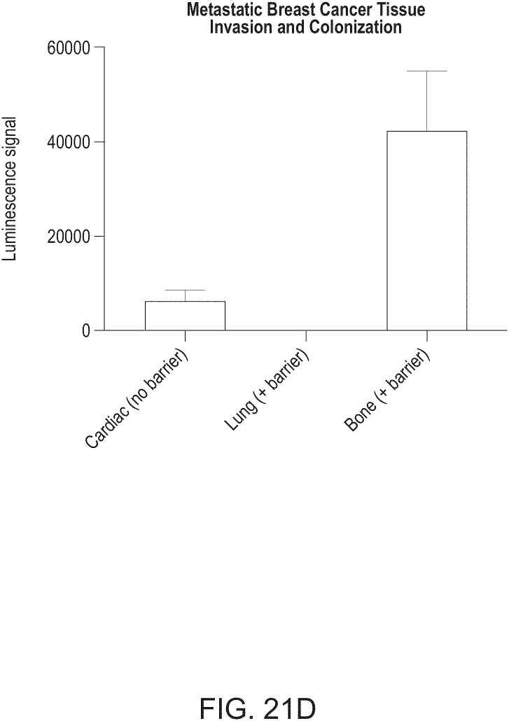

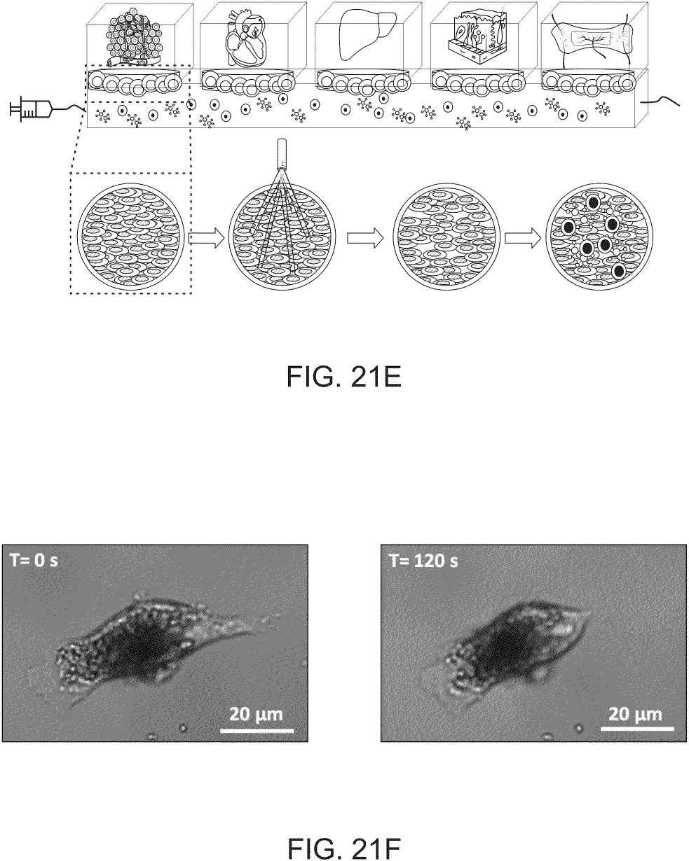

Manipulating permeability of tumor endothelium: FIGS. 21A-F show aspects of the endothelial barrier. FIG. 21A shows an exploded view of the integrated bioreactor containing tissues inserted above the endothelized elastic membrane and cultured over a microfluidic channel to enable integration. FIG. 21B shows a schematic of the integrated platform with tissue specific media in each chamber and FIG. 21C shows the common medium under the vascular barrier. FIG. 21D shows metastatic and luminescence-tagged MDA-MB231 breast tumor cells introduced into the platform with luminescence signal of each tissue measured after 2 weeks to quantify metasthesis. FIG. 21E shows a light controlled multi-tissue platform for studying metastasis and drug screening with tunable endothelial leakiness. Patterned light illumination leads to cytoskeletal organization in endothelial cells and formation of gaps, enabling the exchange of drugs and cells between the tissue compartment and the perfused channel. FIG. 21F shows that blue light illumination of HUVECs expressing Cry2-RhoA leads to cytoskeletal reorganization and membrane retraction within 2 minutes.

We can incorporate spatiotemporal control of endothelial permeability via a RhoA pathway optogenetic switch (FIGS. 21A-C, E). RhoA plays major role in blood vessel permeability, by controlling cytoskeletal organization and thus endothelial cell gaps. ECs expressing A. thaliana Cry2 fused to RhoA can provide light-control of endothelial permeability. Cry2 rapidly oligomerizes upon blue light activation, leading to RhoA activation (GTP-bound), which is fully reversed by keeping cells in the dark. Blue light illumination (488 nm) for 2 min induces EC contraction (FIG. 21F). We can use light-sensitive ECs to form confluent endothelium. To achieve the heterogeneous blood vessel permeability observed in tumors, we can use the system we developed for precise, patterned illumination at varying light intensity, allowing independent fine tuning of endothelial permeability in different tissues (FIGS. 21A-F). Alternative approaches (e.g., thrombin, TNF-alpha, VEGF) are limited in their ability to control cell gap size, have systemic effects, and induce non-reversible permeabilization.

Validation of tumor metastasis phenotypes: Cells that successfully migrate from an engineered tumor to host tissues can be collected and characterized via single-cell (sc) RNAseq, to serve as an input for elucidation of novel targets and pathways, and for predicting therapeutic treatment options in method of use 3. Investigating the cellular subpopulation most likely to metastasize, via track-labeling and scRNASeq profiling in the engineered platform, can enable characterization of “pro-metastatic” cell phenotypes within the heterogeneous populations derived from the tumor. Metastases in the platform can be similarly characterized and by comparisons with the patient-matching clinical samples. Circulating secretome can be analyzed at different time points - when tumor and hosts are first integrated, during intravasation, tumor cell circulation, extravasation and colonization, to identify possible signaling events related to tumor EVs involved in metastatic progression.

Method of use 3: Elucidate master regulators and predict drug sensitivity in metastatic cells using the “cancer patient on a chip” model

The method includes (1) Master Regulator (MR) analysis using scRNASeq signatures of the engineered primary tumors, CTC in vascular flow, and tumor metastases in host tissues, to identify (i) MR proteins that mechanistically determine tumor cell priming for metastatic progression and (ii) unique dependencies of CTC and metastatic cells. (2) OncoTreat analysis using the same scRNASeq signatures to identify small molecule inhibitors capable of reversing the activity of subpopulation-specific MR proteins to induce cell demise or reprogramming to a non-invasive state. (3) Experimental validation of these findings in the “cancer patient on a chip.”

To assess clonal heterogeneity, we can systematically authenticate tumor tissues (primary and metastatic) cultured in the bioengineered platform, at the single cell level. Specifically, we can compare gene expression and MR-activity profiles of single cells and bulk exomes from bioengineered tumors and surgical specimens, across three compartments: (a) primary tumors (method of use 1), (b) CTCs - infused or intravasated from the primary tumor compartment, and (c) bone, lung and liver metastases (method of use 2). Due to metaVIPER’s ability to effectively reduce batch effects and other technical artifacts, these analyses can help identify cross-compartment subpopulations of related cells, by MR overlap analysis. For instance, we can use meta VIPER to identify primary tumor cells that most closely match to CTCs and metastases, assess potential priming for metastatic progression, and validate by lineage-tracking. In addition, cells from all three compartments can be analyzed by OncoTreat to identify drugs and drug combinations most likely to invert MR protein activity, thus inducing cell demise or reprogramming to a non-invasive state (method of use 3). Predictions can be tested in the engineered platform over 4-12 weeks of culture, by assessing drug-mediated depletion of cellular compartments and overall reduction of metastatic cells in target tissues. We can thus be able to isolate and study the individual processes that comprise metastatic progression, identify critical cell subpopulations manifesting sensitivity to orthogonal treatments, and test panels of drugs predicted by scRNASeq. We envision offering this platform for broad use in studies of human cancer, and demonstrating its utility, predictability and reproducibility.

To characterize the biological relevance of bioengineered tumors from method of uses 1 and 2, tissue from both primary and metastatic site can be dissociated, exome/RNA-Sequenced, and compared to patient-matched surgical samples. To demonstrate the utility of the “cancer patient on a chip” for systems biology studies, we can experimentally test OncoTreat-predicted drug responses as inferred by meta VIPER.

Large-scale single-cell analysis for tumor model validation, tracking metastatic populations and inferring drug responses. Throughout the proposed studies, we can take advantage of recent advances in large-scale scRNASeq for three main purposes: (a) Validating and studying primary tumor models (OS and breast cancer); (b) Analyzing metastases in host tissues (bone, liver, lung); and (c) Inferring single-cell-specific drug sensitivity for primaries, CTCs, and metastases by OncoTreat analysis. A computer- controlled microfluidic platform for scRNASeq, ideally suited to complex tumors analysis can parallel-profile ~ 10,000 cells with rapid, high-efficiency cell capture (>50%), using optical microscopy for on-chip analysis of markers, cell viability, and cell lysis. The device has a simple flow cell with an array of microwells on a glass slide and is fabricated by soft lithography. In a typical experiment, cells are gravity- loaded, one cell per microwell, followed by loading of polymer beads coated with oligonucleotide primers that contain a universal adapter sequence, a bead-specific barcode sequence, a unique molecular identifier (UMI), and oligo(dT) for mRNA capture. Cell lysis and reverse transcription occur automatically, following introduction of strongly denaturing lysis buffer and a perfluorinated oil to seal the array. On-chip fluorescence imaging facilitates control of cell viability, lysis, sealing, and counting. Following mRNA capture, we introduce a detergent-containing buffer to rapidly remove sealant and lysate, such that barcoded beads with hybridized mRNA are exposed, allowing reverse transcription to occur automatically. cDNA-coated beads are pooled, harvested, and 3′-end RNA-Seq libraries are generated using the SCRB-Seq protocol and the Nextera transposition system (Illumina), at a cost as low as $0.05/cell.

We sequence the pooled libraries with paired-end sequencing, where the first read contains both the cell- identifying barcode and a unique molecular identifier (UMI) barcode, and the second read contains transcript sequences. For the proposed studies, we can align read two to the human genome using the STAR aligner with a transcriptome annotation, to take advantage of its splice-awareness. By combining the alignment with the forming barcode in the first read, we assign an address to each read that aligns uniquely to a locus that can be unambiguously assigned to an annotated gene. Given the strand-specific nature of the libraries, strand information can be used to ensure proper alignment. The address includes the read ID, cell-identifying barcode, UMI, and gene symbol. This algorithm gives us an estimate of the number of captured mRNA molecules associated with each gene in each cell.

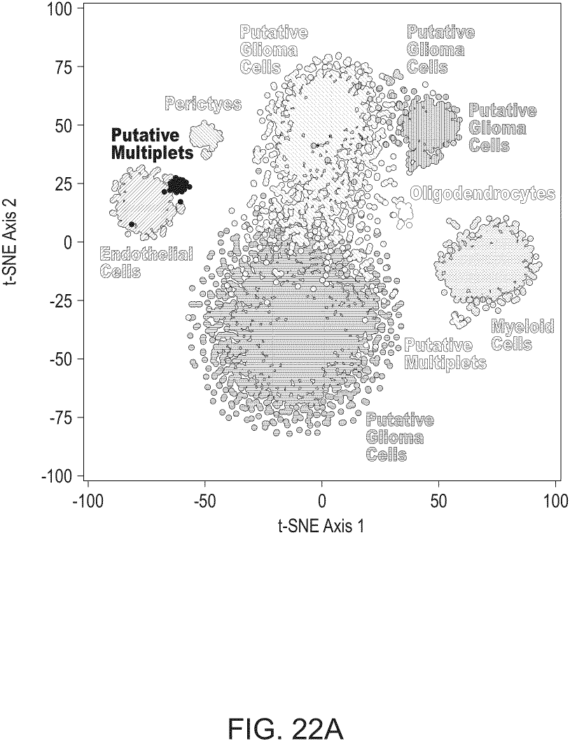

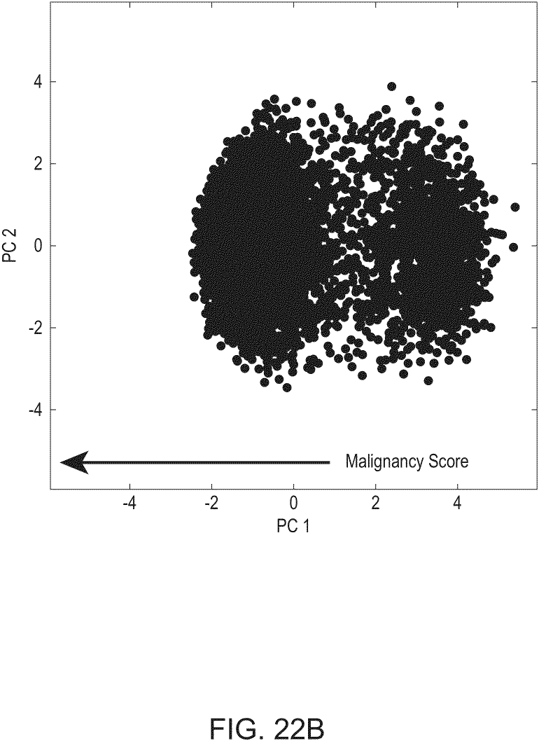

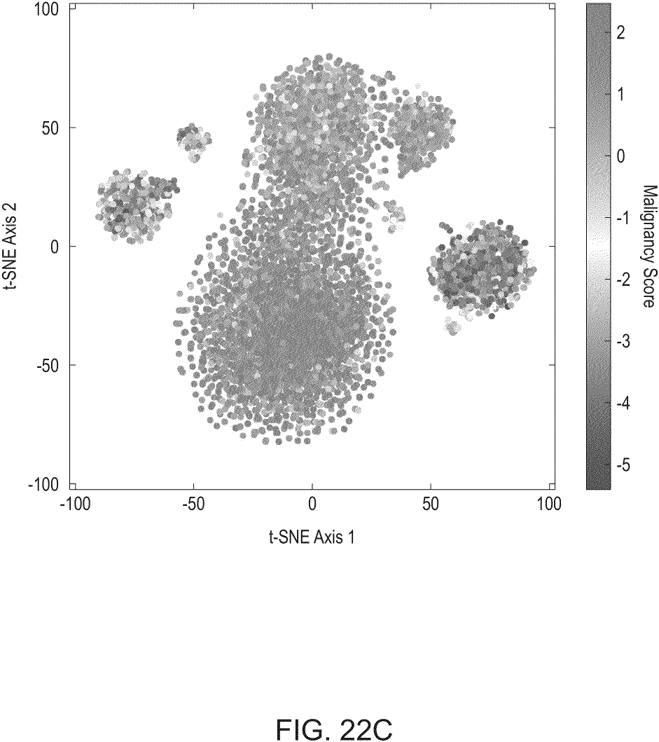

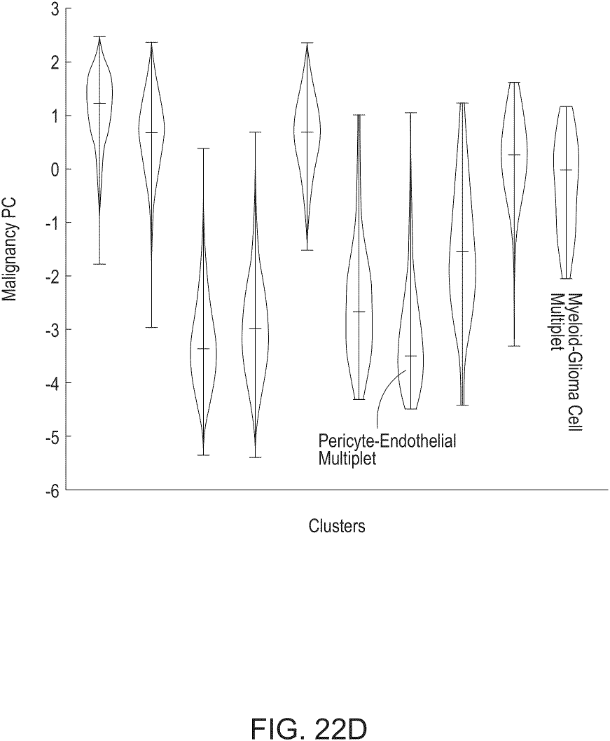

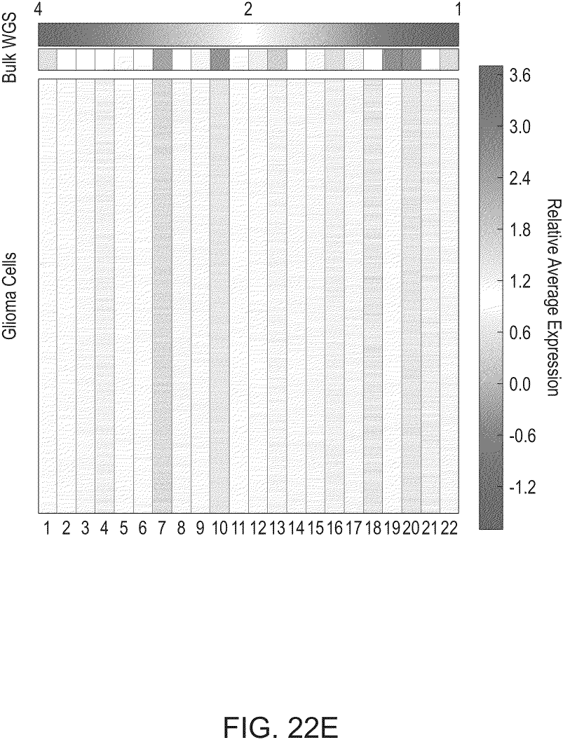

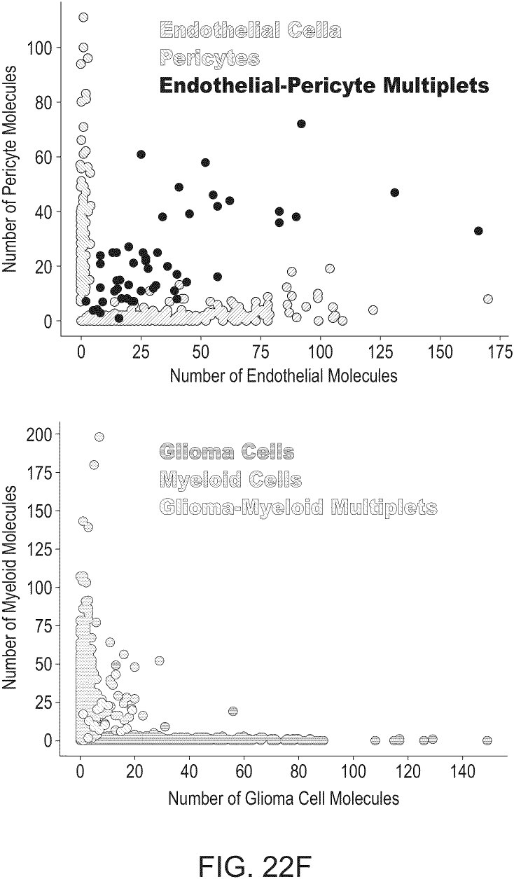

Application of our technology to a large-scale analysis of human glioblastoma (GBM) surgical specimens yielded a number of interesting findings. Because transformed glioma cells in GBM typically resemble glia at the level of gene expression, identifying malignantly transformed cells from scRNASeq data is non-trivial, and this is an issue we can face in the proposed studies. Fortunately, previous studies have shown that large copy number variants (CNVs) and aneuploidies are readily detectable by scRNASeq of tumor tissues. Principal component analysis (PCA) of the chromosomal expression matrix for each patient consistently revealed an axis of variation that separated putatively transformed cells, which harbor known expression patterns associated with GBM, from those that expressed common markers of cells in the microenvironment (FIGS. 22A-F). We call this axis the malignancy score. To validate this score, we conduct low-coverage whole genome sequencing (WGS) of bulk tumor tissue from each patient and compute the average copy number of each chromosome by comparison to a diploid reference. CNVs evident in bulk WGS were in good agreement with the most prominent alterations in scRNASeq data (FIGS. 22A-F). This analysis gives us confidence that the transformed populations of cells are indeed mutated.

FIGS. 22A-F show examples for the application of large-scale single-cell RNA-Seq to surgical specimens of human tumors. FIG. 22A shows a tSNE projection showing graph-based clustering of scRNA-Seq gene expression data of glioma tumor cells, with identification of putative tumor cells, non-neoplastic cells and putative multiplets. FIGS. 22B and 22C show PCA projections of scRNA-Seq chromosomal expression of the same tumor showing two major clusters. One overlaps with the putative transformed cells (FIG. 22B) and the other overlaps with the remaining cells (FIG. 22C). FIG. 22D shows a distribution of the malignancy scores among cells in each cluster in FIG. 22A. An asterisk marks each putatively transformed cluster FIG. 22E shows chromosomal copy numbers for bulk WGS of the same tumor, and relative expression of each chromosome in each putatively transformed cell. Major features such as chr7 gain and chr10 loss are recapitulated by the scRNA-Seq data, confirming the mutated status of the putatively transformed cells. FIG. 22F shows barnyard plots showing that the putative multiplet clusters are, in fact, multiplets (from endothelial-pericyte in the top case and from glioma-myeloid in the bottom case.

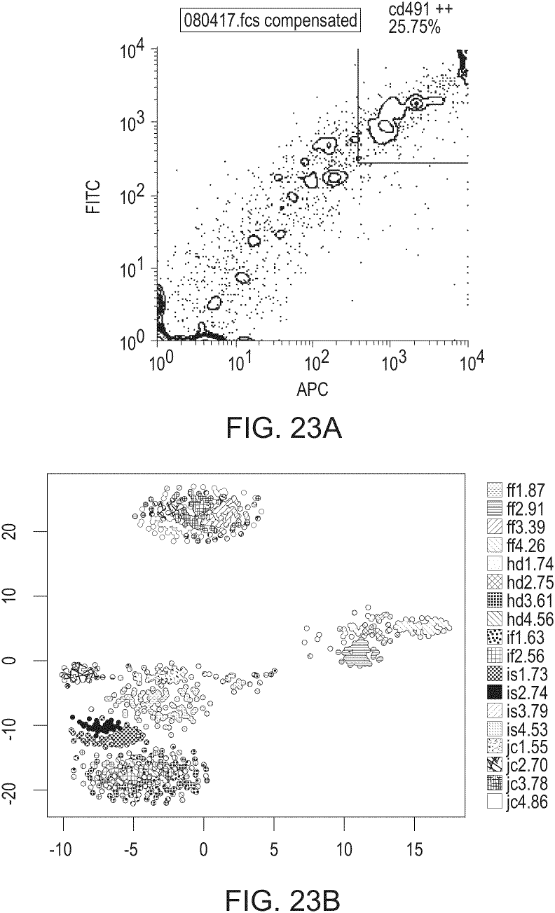

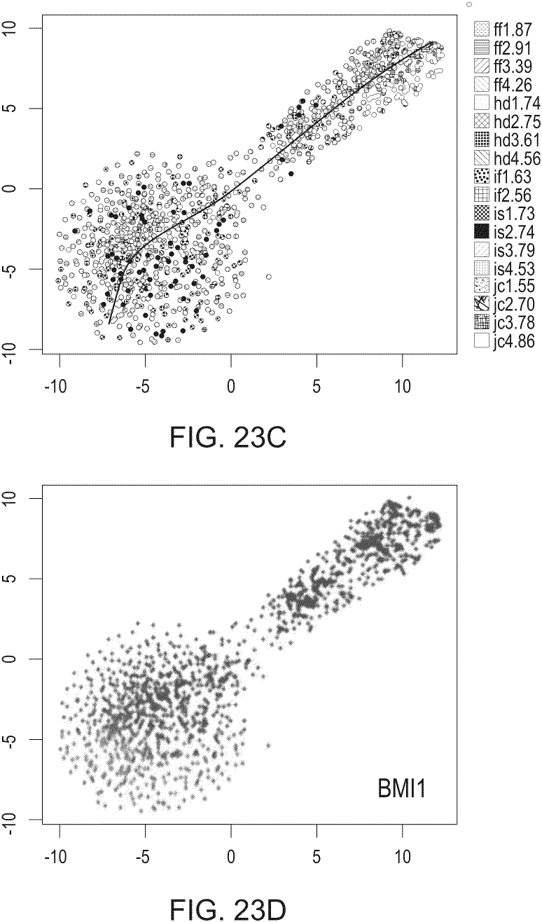

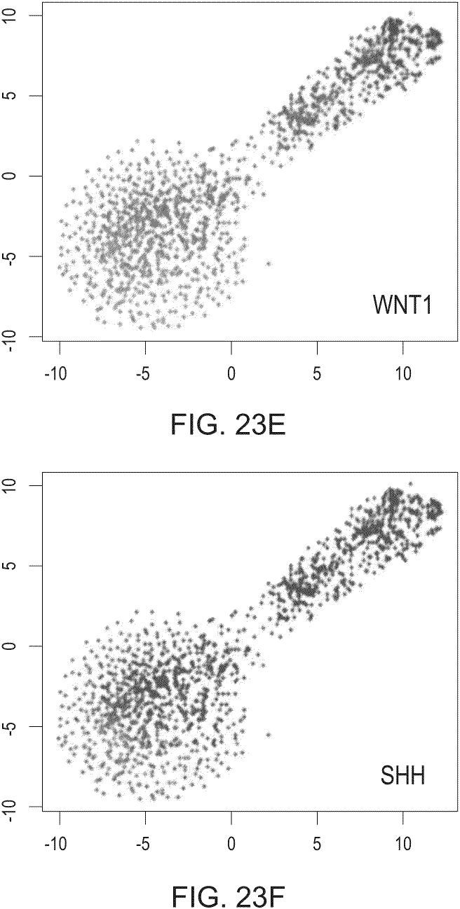

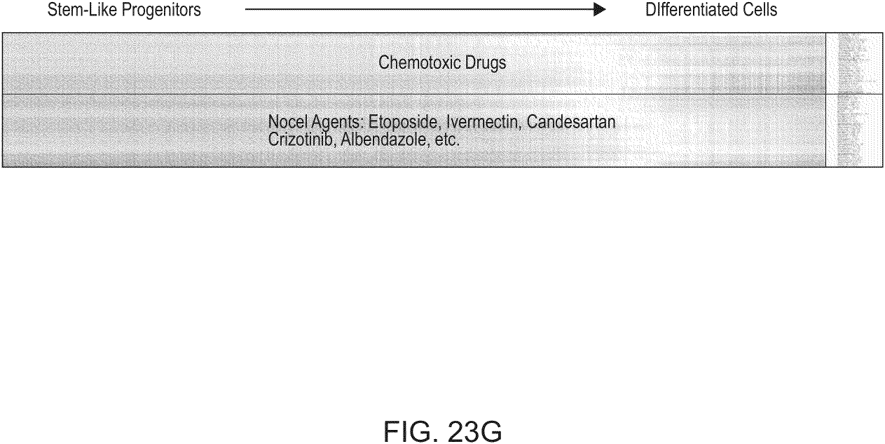

FIGS. 23A-G show aspects of breast cancer bioengineering. FIG. 23A shows that CD49+/Epcam+ cells from 5 breast cancer patients were FACS sorted and profiled on our well-based platform. 25% unsorted cells were also selected and profiled FIG. 23B shows that t-SNE clustering of gene expression profiles show a significant patient-specific batch effect FIG. 8C shows that) t-SNE clustering of metaVIPER-inferred protein activity profiles effectively removes the batch effect FIGS. 23D - F show activity of previously established markers of breast cancer stem-like progenitor cells, BMI1, WNT1, SHH and NOTCH1 (not shown). They show clearly differentiated cells (blue) and progenitor like cells (red). About 5% of cells are positive for all 4 markers and represent stem-like progenitors. FIG. 23G shows OncoTreat prioritized drugs have completely orthogonal activity in progenitor and differentiated breast cancer cells (blue indicates statistically significant cell state MR activity reversal; red indicates further increase in MR activity). Columns represent ~500 cells from 5 patients, rows represent top predicted drugs for the two subpopulations. Two drugs identified by this analysis (ivermectin and etoposide) were identified in a previous breast cancer stem cell screen. Other drugs are currently in validation.

Similarly, metaVIPER-inferred protein activity profiles successfully traced differentiation of single cells from five distinct breast cancer patients from their breast cancer stem-like progenitor (BCSLP) state to a fully differentiated one (FIGS. 23A-C). BCSLP markers, including SHH, NOTCH2, WNT1, and BMI1 were shown to track along the lineage trace (FIGS. 23D-F). We also adapted the OncoTreat algorithm to identify drugs that revert MR protein activity on an individual cell basis (FIG. 23G). Consistently, single cell OncoTreat analysis identified promising drugs, including some previously identified in BCSLP screens. Based on our significant experience with surgical specimens, including breast cancer, we can target 3,000-5,000 cells/sample at ~50,000 reads/cell, and rely on extensive QC assessments at each step in the procedure. For instance, cell concentration, viability, and debris can be assessed by an automated cell counter and bright field microscope. cDNA length distribution and yield can be assessed by Bioanalyzer, as proxies for RNA quality and molecular capture efficiency, to avoid sequencing low-quality samples.

Testing of OncoTreat predicted drugs in the patient-specific engineered platform: The bioengineered primary tumor (OS, TNBC, ER+) connected by vascular flow with bone, lung, liver and heart tissue can be used to study activity of OncoTreat predicted drugs. We can focus on drugs targeting progression steps from each compartment. Specifically, we can analyze differential expression signatures between CTCs and primary cells, between metastatic and primary cells, and between metastatic cells and CTCs, using metaVIPER. Cell signatures can be generated between the subpopulations in each compartment and the closest-matching subpopulation in earlier compartments, based on MR protein overlap (e.g., a CTC subpopulation and its closest match in the G primary tumor). This can identify drugs targeting MR associated with metastatic progression processes, as shown in. Standard approaches for measuring dose response curves in the engineered system, in target and control cells can be used to test drug activity.

We tested the drug Linsitinib—a potent tyrosine kinase inhibitor targeting the IGF-1 receptor (IGF-1R) —in metastatic and non-metastatic OS models (FIG. 17C). The drug was administered (12 µM, for 3 weeks), as per an ongoing Phase II Eurosarc trial in patients with advanced Ewing sarcoma. Gene expression analysis of bioengineered tumors showed upregulation of IGF-1R and INSR, the targets of Linsitinib. Following treatment, the tumors engineered from non-metastatic OS cells showed significant viability reduction, compared to tumors from metastatic cells, where, consistent with clinical results, the drug had no effect.

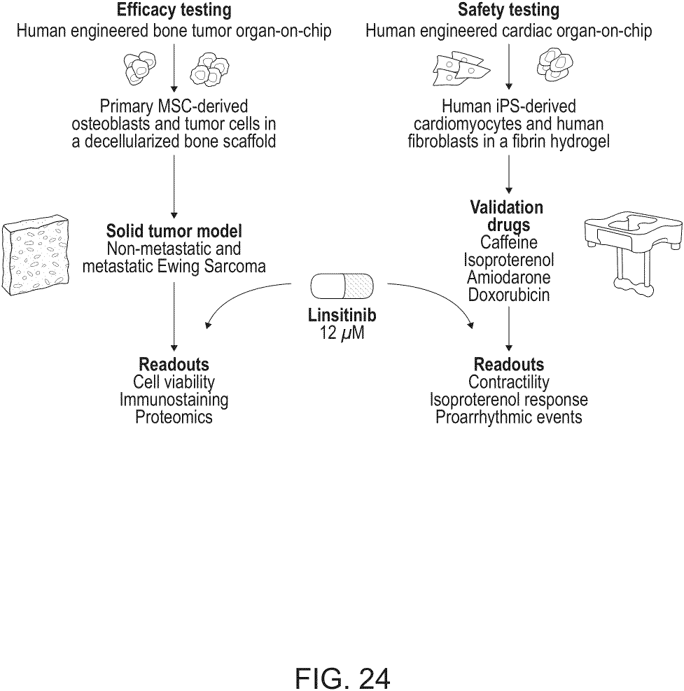

We developed two different organs-on-chips to evaluate anti-cancer drug efficacy (using a bioengineered human Ewing sarcoma tumor) and cardiac safety (using a bioengineered human cardiac tissue). Both organs were generated with human cells and characterized and validated before being exposed to linsitinib, a novel anti-cancer therapeutic agent, in isolated culture and within the novel platform with microfluidic perfusion. FIG. 24 shows aspects of the study design.

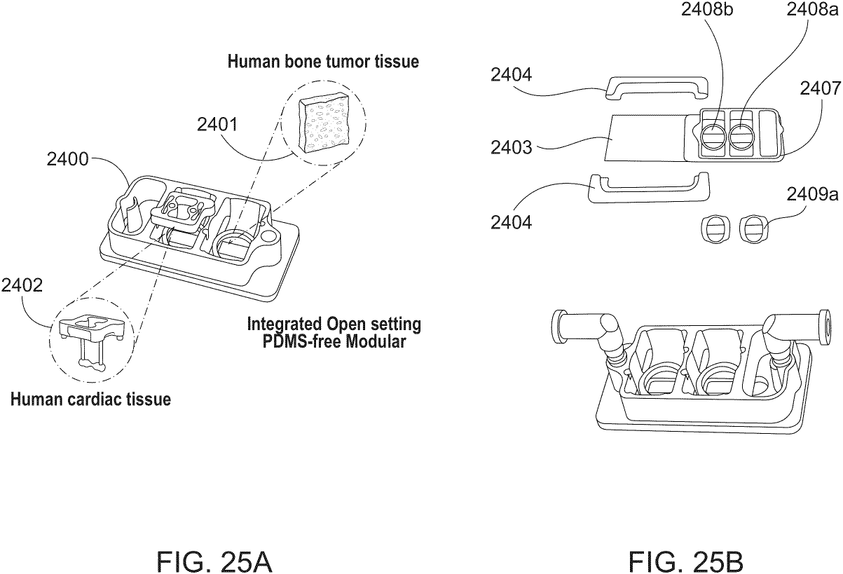

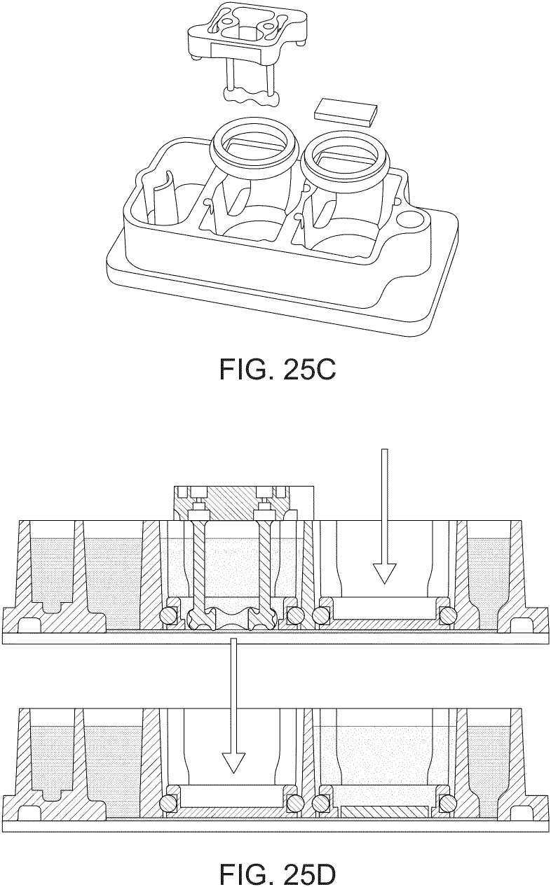

In another embodiment of the system, a PDMS-free, modular and integrated two-tissue system 2400 is provided. FIG. 25A shows a schematic of the system comprising two engineered human tissues: Ewing sarcoma (ES) tumor 2401 and cardiac tissue 2402) that were cultured either with microfluidic perfusion (integrated platform) or in isolation. Metastatic and non-metastatic ES tumors were studied at clinical dosages and treatment regimens of linsitinib. FIG. 25B shows photographs of the system and its components (top) and in its complete functional state (bottom). Referring to FIG. 25B (top) the system includes a platform, first and second tissue culture chambers 2408a and 2408b (as described above with respect to FIG. 1B), a plate 2403, housing 2404 and insertable devices 2409a and 2409b. FIG. 25C shows assembly of the system. When assembled platform 2407 sits on plate 2403 and housing members 2404a and 2404b slide onto plate 2403 and platform edges 2407 to secure the platform to the plate. Tubular microfluidic 2410a and 2410b connections are secured to the entrance and exit ports 2412a and 2412b on platform 2407 as described above to permit circulation at the opposing ends of the system. A reservoir is disposed in the platform for perfusate. FIG. 25D shows the system setup for culturing tissues in isolation, as shown for the cardiac tissue (top) and the bone tumor tissue (bottom). Arrows indicate polypropylene plugs (or insertable devices) isolating that chamber from the rest of the system, allowing to culture in isolation our tissues.

In one embodiment, the system has four main components: (i) the platform with tissue chambers and medium reservoir, (ii) first and second housing clamps, (iii) an o-ring, and (iv) a glass slide or plate at the bottom (FIGS. 25A-B). The open setting of the platform allows manual sampling, and the glass slide allows microscopic analysis. Each tissue is cultured in its own chamber, the bottom of which includes a nylon mesh with 20 µm pores (FIG. 25C). These inserts can be replaced by polypropylene plugs when the tissues need to be cultured in isolation (FIG. 25D). Underneath the nylon mesh membrane, the tissues are connected through a channel that runs along the length of the platform 2407, connecting the flow inlet, the individual tissue chambers, the reservoir where drugs can be introduced, and the flow outlet.

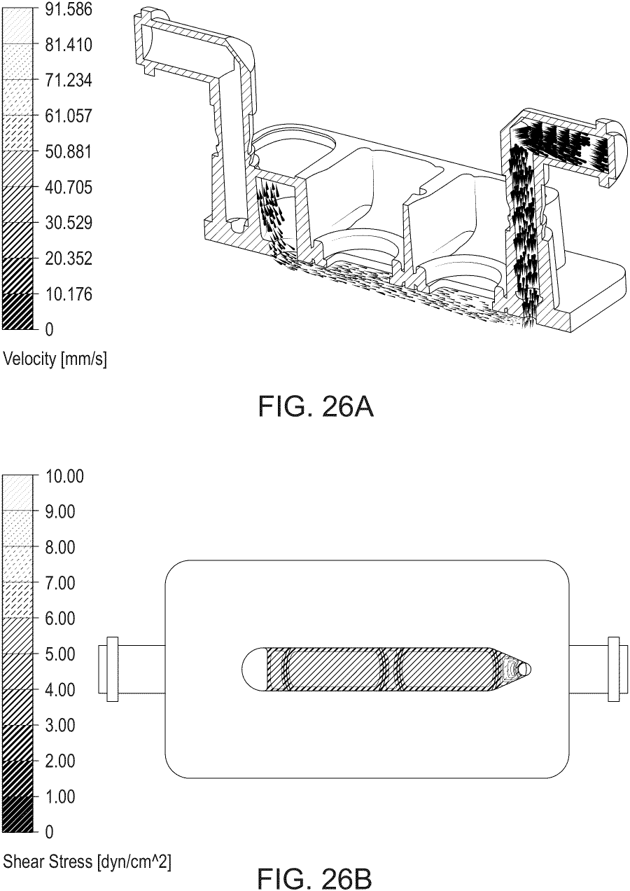

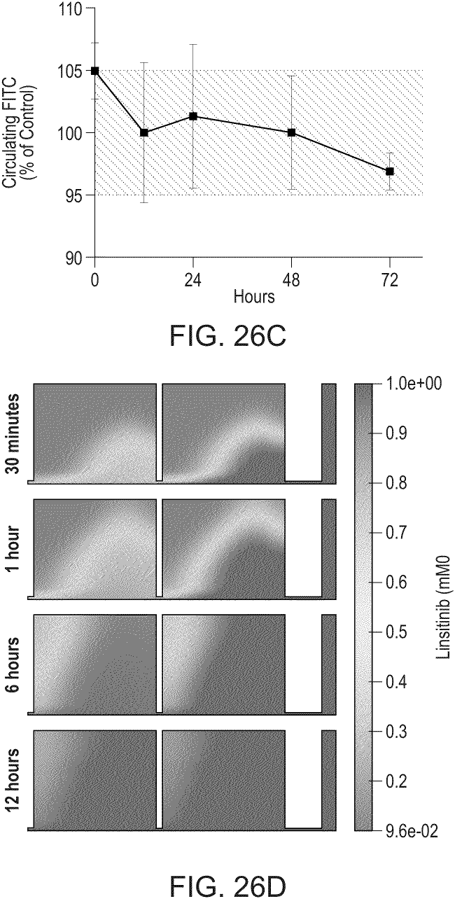

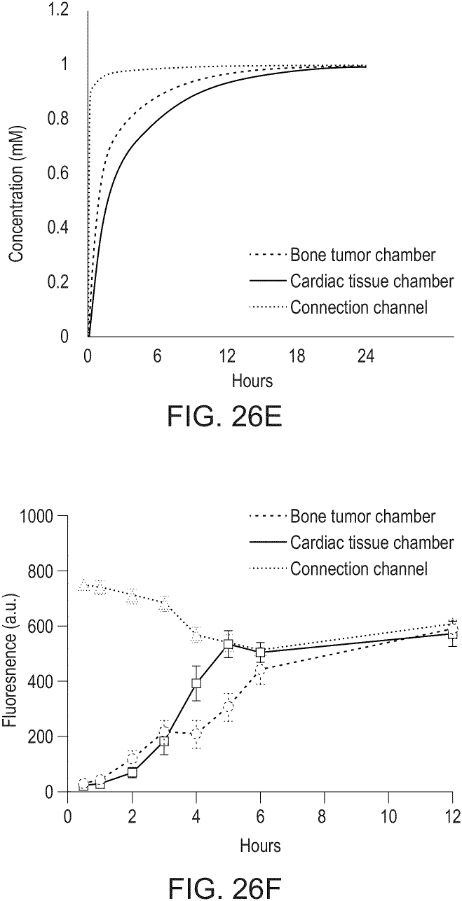

FIGS. 26A-F show aspects of concentration profiles of a hydrophobic small-molecule tracer and linsitinib circulation within the system. FIG. 26A shows simulated fluid flow velocity of circulating medium in the system. FIG. 26B shows simulated shear stress. FIG. 26C shows that hydrophobic FITC (10 µM) was circulated in the system and its concentration, relative to a control sample in a multi-well or multi-chamber platform, was assessed at 12, 24, 48 and 72 hours (mean ± s.e.m., n = 3). FIG. 26D shows simulated linsitinib concentration gradients within each tissue chamber at 30 min and 1, 6 and 12 hours after introduction of linsitinib to the media reservoir. FIG. 26E shows simulated linsitinib concentration in both tissue chambers and in the microfluidic channel over 24 hours. FIG. 26F shows empirical FITC concentrations across both individual tissue chambers and the microfluidic channel was measured every 2 hours for up to 12 hours (mean ± s.e.m., n = 4).

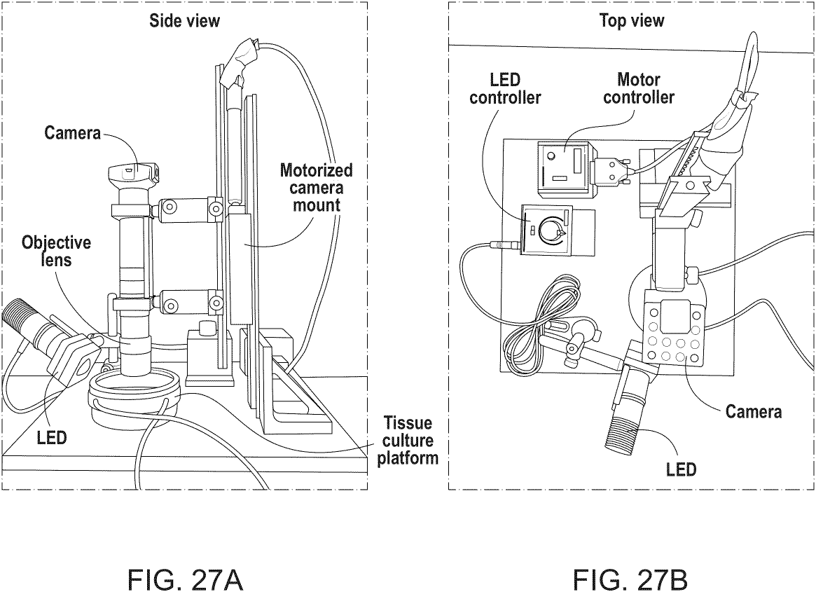

The system may utilize a single channel of a peristaltic pump to recirculate the media at a desired flow rate and shear stress (FIGS. 26A and 26B, FIGS. 27A-C), within the physiological range for human capillaries. Design details are summarized in the table below.

| | |
|---|---|
| Length | 60 mm |
| Height | 20 mm |
| Width | 26 mm |
| Height of the connection channel | 0.3 mm |
| Length of the connection channel | 5 mm |
| Overall diameter of transwell | 12 mm |
| Porous mesh area of transwell | 8×4 mm |
| Transwell pores | 20 µm |
| Circulating volume | 5 mL |
| Chamber volume | 1.5 mL |
| Flow rate | 3.3 mL/min |
| Shear stress | 5 dyn/cm² |

The system sterility was confirmed by 4-week incubation with soybean casein digest medium that is specific for the growth of aerobic bacteria and fungi.

In some embodiments, the central piece of the bioreactor platform is made of polysulfone, which is a tough, stable, and biocompatible thermoplastic polymer that does not absorb hydrophobic molecules and is being used for the fabrication of new organ-on-a-chip platforms. Fluorescein isothiocyanate (FITC), a small molecule, hydrophobic, fluorescent dye, was circulated for 72 hours, without any measurable absorption by the platform, as compared to multi-well plates (FIG. 26C).

The computational fluid dynamics software CoBi was used for simulations of linsitinib transport across the porous nylon mesh membranes separating the individual tissue chambers and flow channel. CoBi has been used previously to simulate drug analog transport in the eye and air flow in the lung.

We found that linsitinib introduced into the circulation at a 3.3 mL/min flow rate reached uniform concentration between the connection channel and both tissue chambers within 12 hours, and that it diffused into the tissues within 6 hours (FIGS. 26D-E).

We also circulated fluorescent FITC, which has similar chemical properties as linsitinib including hydrophobicity and molecular weight, and measured its distribution across the 2 tissue chambers in the platform (FIG. 26F). The simulated and empirical results agreed: FITC also reached uniform concentration in all areas of the platform in within 12 hours, after reaching equilibrium across both models at approximately 6 hours. This is significant, since linsitinib is known to have a short half-life of approximately 5 hours. The delayed drug distribution by diffusion through tissues observed here has been documented as an issue for treating solid tumors in patients, with chemotherapeutic concentrations decreasing exponentially with distance from tumor blood vessels and often being limited to the peripheries of tumors even 12 hours after injection.

In the platform, the tissues are cultured with a transwell located at the bottom of the chamber. Because of the location of the transwell, it was difficult to visualize the tissue with the inverted microscope we had available in our lab. Thus, we created an in-house microscope with an upright objective (Mitutoyo Inc., Magnification: 2X) and a working distance of 34 mm allowing visualization of the tissue (FIGS. 27A and 27B). Side (FIG. 27A) and top (FIG. 27B) views of the microscope system show that that is constructed using commercially available optomechanical components. To image the tissues in the platform, we incorporated a 2X microscopic objective lens (Mitutoyo Inc.) with a long working distance (34 mm) into the imaging system. The exchangeable LED light source allows both bright-field and fluorescent imaging. The motorized camera mount enables precise focus of different imaging planes across the tissue surfaces. By incorporating appropriate optical filters and light sources, this system also enables fluorescent imaging of the tissue.

### Validation of Engineered Ewing Sarcoma and Cardiac Tissue Models

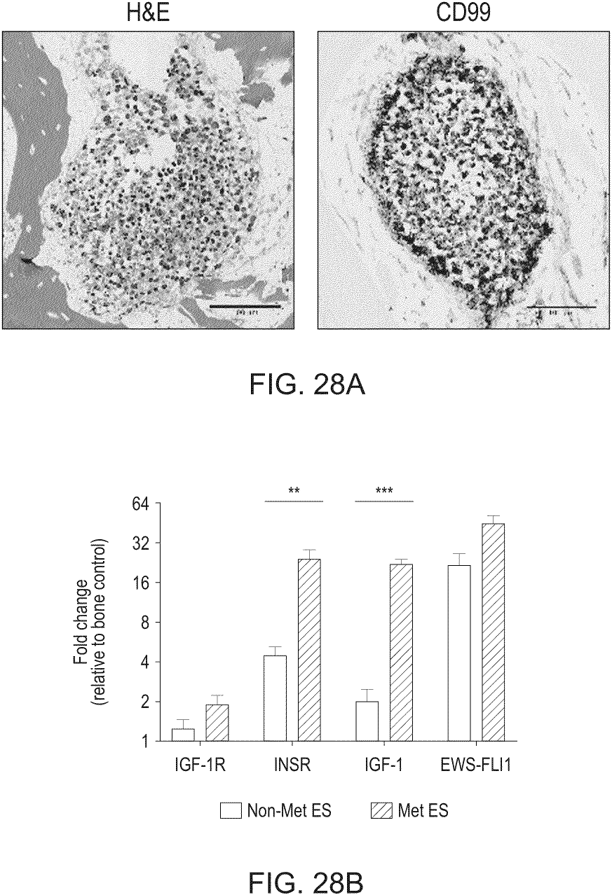

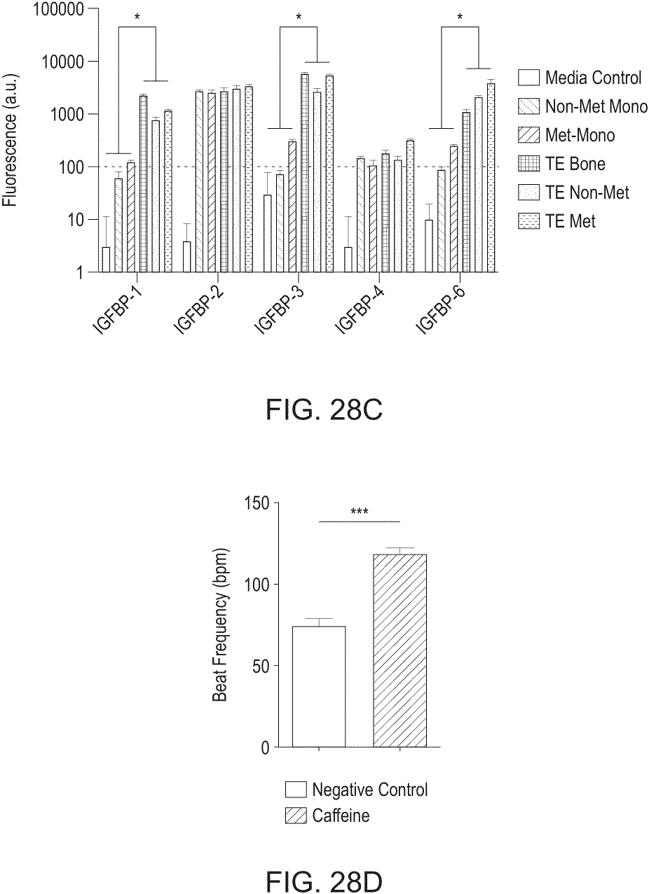

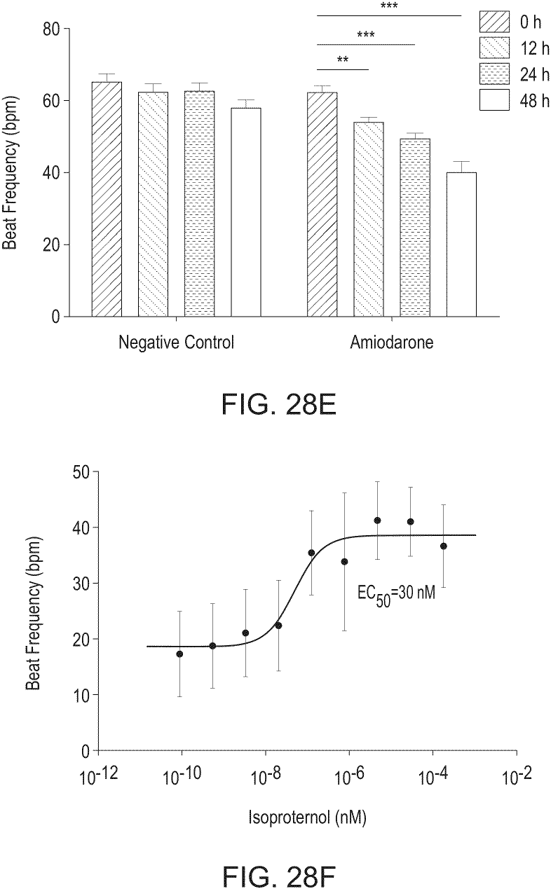

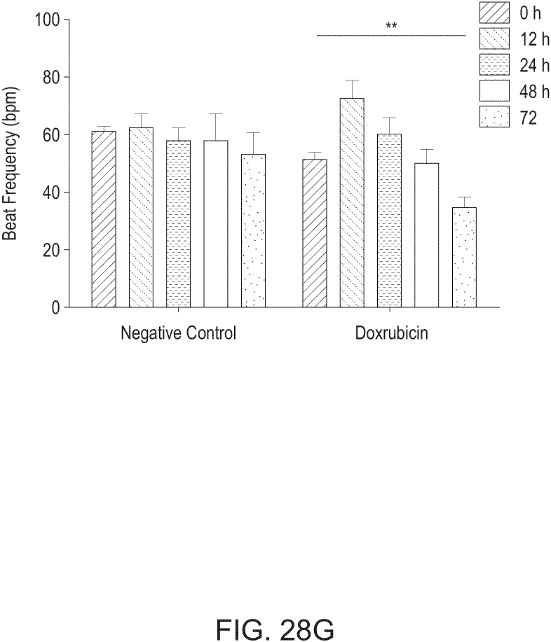

FIGS. 28A-G show aspects of the development and validation of the engineered human Ewing sarcoma (ES) bone tumor and human cardiac tissue. FIG. 28A shows the immunohistochemistry analysis of the engineered tumor tissues. H&E staining demonstrates tumor morphology within the tissue engineered bone, and positivity for ES marker CD99. FIG. 28B shows gene expression of ES translocation marker EWS-FLI1 and linsitinib targets in non-metastatic and metastatic ES engineered tissues. Levels were normalized first to β actin and subsequently to the tissue engineered bone control (mean ± s.d., n = 3). FIG. 28C shows the proteomic analysis of IGF-1 binding proteins secreted by tumor cells grown in monolayer as compared to our engineered bone (control) and bone tumor tissues (mean ± s.d., n = 3). FIG. 28D shows the human engineered cardiac tissue response to caffeine (50 mM) (mean ± s.e.m., n = 5). FIG. 28E shows the human engineered cardiac tissue response to amiodarone (2.418 µM) over 48 hours (mean± s.e.m., n = 6 for negative control; n = 7 for amiodarone]). FIGS. 11F shows an isoproterenol dose-response study of engineered cardiac tissues (mean ± s.e.m., n = 63). FIG. 28G shows the response of cardiac tissues to doxorubicin (1 µM) over 72 hours (mean± s.e.m., n = 7). *P < 0.05; **P < 0.01; ***P < 0.001, by two-way ANOVA with Bonferroni post-test or unpaired two-tailed Student’s t test. All scale bars: 100 µm.

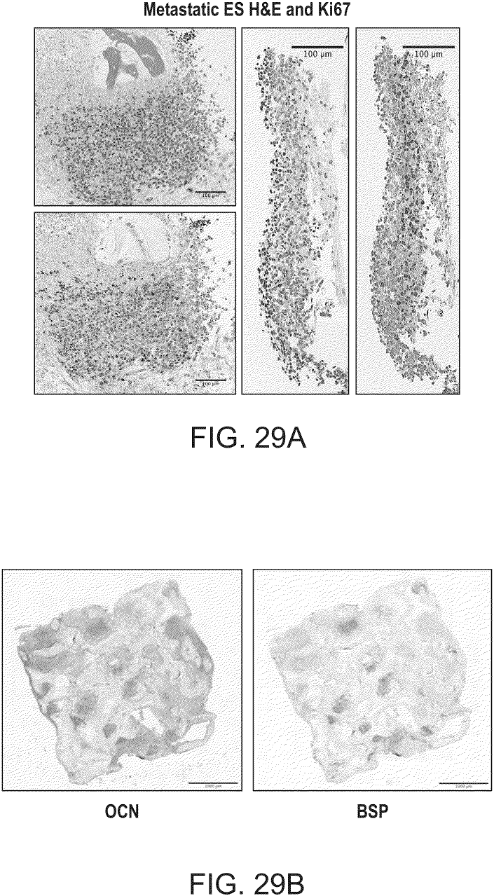

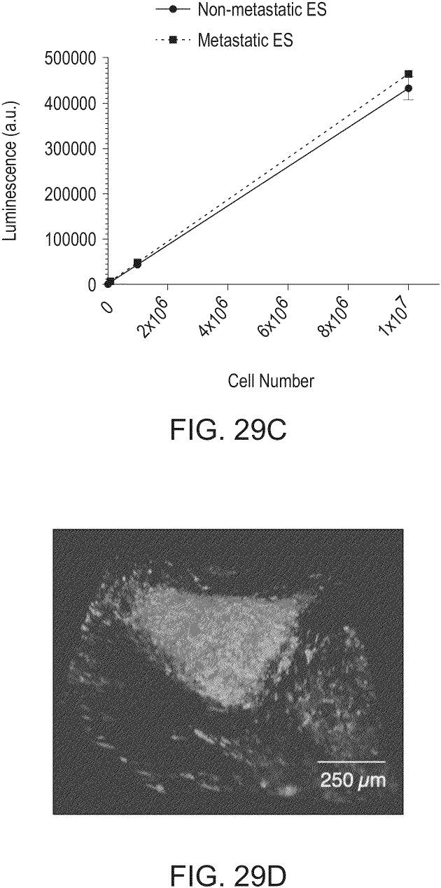

FIGS. 29A-D show aspects of immunohistochemical (IHC) staining of engineered ES tumors with transduced GFP-luciferase positive cancer cells. FIG. 29A shows H&E staining and parallel IHC analysis of proliferation marker Ki67 of the metastatic ES engineered tissue. Variability in tumor morphology and the numbers of proliferative Ki67 positive cancer cells demonstrates ability to recreate some degree of intra- and inter-tumor heterogeneity. FIG. 29B shows IHC staining of engineered bone tumors showing positivity for mature, functional osteoblast markers osteocalcin (OCN) and sialoprotein (BSP). FIG. 29C shows that monolayers of each of the transduced and sorted GFP-luciferase positive ES cancer cell lines (non-metastatic and metastatic) were formed (using 10⁴, 10⁵, and 10⁶ cells per well) and the luminescence signal was measured (mean± s.e.m., n = 3) to determine if it could serve as an effective function of cell number and viability. FIG. 29D shows a confocal microscopy image of a GFP-luciferase positive engineered bone tumor in situ on the engineered human bone tissue, mineral bone scaffold. All scale bars: 100 or 250 µm, as noted.

Both types of primary ES tumor cells for our models: metastatic (SK-N-MC cell line) and non-metastatic (RD-ES cell line) maintained their native-like tumor morphology and expression of the ES cell marker CD99, following cultivation within engineered bone tissues (FIG. 28A). Monolayer cultures of ES cells fail to model tumor morphology and heterogeneity. In contrast, we observed a variation of tumor sizes and locations across the bone tissue (i.e. inter-tumor heterogeneity) as well as intra-tumor heterogeneity, with ES cells showing variable staining for the proliferation marker Ki67 (FIG. 29A).

Gene expression analysis by qRT-PCR of linsitinib target IGF-1R in TE-ES models revealed levels similar to those in engineered bone controls (FIG. 29B). Unlike tissue engineered tumor models, tumor cell monolayers do not allow testing of the drug target expression in the surrounding cells, in this case IGF-1R expression in osteoblasts. In addition, significantly higher expression of the insulin receptor (INSR) and the receptor ligand IGF-1 were observed in the metastatic than non-metastatic TE-ES models (FIG. 29B). Given the role of the insulin receptor in developing resistance to IGF-1R inhibitors and the role of the IGF-1 ligand in activating this tumorigenic pathway, these differences between the models may explain why metastatic ES was unresponsive clinically.

Both in the bloodstream and in the tissues, the IGF binding protein (IGFBP) family has a high affinity for the IGF-1 ligand, thus being a critical regulator of the IGF-1R signaling pathway. For this reason, any predictive drug studies of IGF-1R inhibitors would need to be conducted at native-like concentrations of these binding proteins. Proteomic analysis of secreted IGFBPs showed significantly higher expression of IGFBP-1, 3, and 6 in both the TE-ES models and engineered bone tissue as compared to the corresponding tumor cell monolayers, which showed only traces of IGFBP (FIG. 29C). These transcriptional and proteomic results are also consistent with our previous studies that showed the importance of native-like morphology and milieu in models of solid tumors for recapitulating the ES tumors, including the upregulation of IGF-1 tumorigenic and anti-apoptotic pathways.

The cardiac tissue model was generated from iPS-derived cardiomyocytes and fibroblasts encapsulated in fibrin hydrogel, as in our previous studies. The tissue was formed between two elastic pillars causing cell elongation and alignment, and subjected to electrical stimulation to synchronously contract and work against the pillars. The tissues were matured over 4 weeks of culture, and validated by exposure to drugs with known cardiac effects.

When exposed to caffeine, an inducer of ryanodine receptor-mediated calcium release with tachycardic side effect, cardiac tissues demonstrated physiologic increases in beat frequency (FIG. 29D). Amiodarone, an antiarrhythmic therapeutic agent used to treat irregular heartbeats by blocking the potassium channel and increasing the effective refractory period, induced the expected decreases in the beat frequency (FIGS. 29). Upon exposure to isoproterenol, a non-selective beta-adrenergic agonist and a gold standard for assessing the ability of a model to recapitulate beta-adrenergic responses, the beat frequency increased, with expected values of EC50 (FIGS. 29).

When exposed to doxorubicin, a chemotherapeutic with known and well documented cardiotoxic side effects (initial sinus tachycardia, supraventricular tachycardia, chronic dilated cardiomyopathy), the beat frequency initially increased, and then decreased following prolonged exposure to the drug (FIGS. 29). The cardiac model was therefore capable of recapitulating the physiological effects observed clinically in patients for all four drugs, including the doxorubicin toxicity.

### Responses to Linsitinib of Engineered Tumors Cultured in Isolation

The Phase II clinical trial of linsitinib administered for 3 weeks at the blood plasma concentration of 12 µM to patients with refractory or relapsed ES served as a basis for this study. To assess the drug efficacy and safety, we studied the engineered tissues under the same drug regimen used in the clinical study. We first confirmed the maintenance of engineered bone tissue environment over the entire duration of tumor maturation and drug treatment (5 weeks).

Immunohistochemical (IHC) staining of TE-ES samples showed sustained expression of functional osteoblast markers osteocalcin and bone sialoprotein (FIG. 29B). In order to track drug responses of ES cancer cell populations within the engineered bone niche, we labeled the metastatic and non-metastatic ES cells using an HIV-based lentiviral system with the CMV-promoter combined GFP-luciferase vector. Cancer cell titrations demonstrated that the GFP-luciferase expression-dependent luminescence signal served as a reliable readout of viable cancer cells (FIG. 29C). We also monitored the tumor aggregates by live in situ imaging within the bone tissue (FIG. 29D).

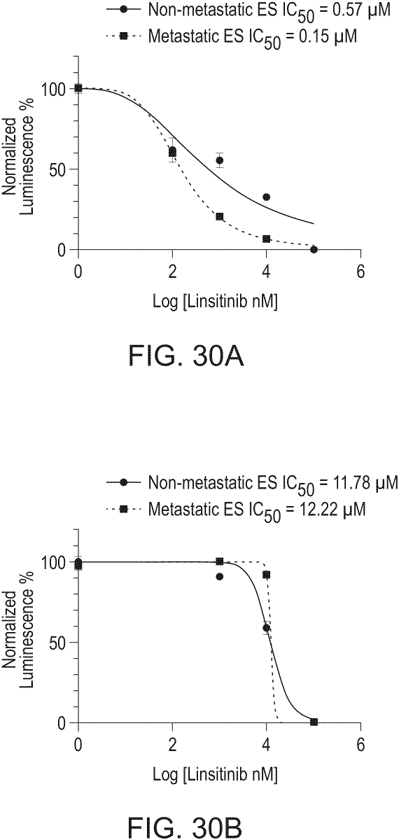

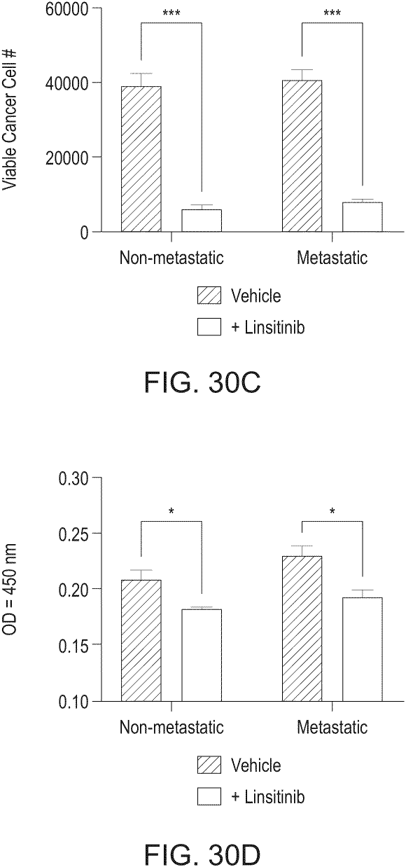

FIGS. 30A-D show aspects of valuation of the dose-dependent effects of linsitinib concentration on metastatic and non-metastatic Ewing sarcoma cell line monolayers. FIG. 30A shows the MTT viability of non-metastatic and metastatic ES cell lines were treated with various concentrations of linsitinib (0, 0.1, 1, 10, and 100 µM) over 72 hours. MTT viability assay was used to determine drug toxicity, with luminescence generated by viable cells measured at the endpoint (10⁴ cells per well, mean± s.e.m., n = 3). FIG. 30B shows results when non-metastatic and metastatic GFP-luciferase transduced, positive ES cell lines were treated with various concentrations of linsitinib (0, 0.1, 1, 10, and 100 µM) over 72 hours. In order to determine cancer cell viability, cells were lysed and treated with luciferin using a Bright-Glo™ assay, with subsequent luminescence measured (10⁴ cells per well, mean± s.e.m., n = 3). FIG. 30C shows results when non-metastatic and metastatic GFP-luciferase transduced, positive ES cell monolayers were treated with 12 µM linsitinib for 72 hours and viability was determined by measuring luminescence of these cells (10⁴ cells per well, mean± s.e.m., n = 3). FIG. 30D shows results when non-metastatic and metastatic ES cell lines were treated with 12 µM linsitinib for 6 hours, lysed, measured for protein quantity using a standard BCA protein quantification assay, and loaded equally onto an ELISA to semi-quantitatively determine phosphorylated levels of the IGF-1 receptor (mean± s.e.m., n = 4). *P < 0.05; **P < 0.01; ***P < 0.001 by unpaired two-tailed Student’s t test.

In ES cell monolayers, MTT viability assay resulted in the IC₅₀ for linsitinib that was two orders of magnitude lower than the effective plasma concentration observed in patients (FIG. 30A). However, when luminescence was used as a proxy for cell viability, the IC₅₀ concentrations for linsitinib were in line with the 12 µM Cₘₐₓ clinical concentration, suggesting the validity of this assay for evaluating tumor cell drug responses (FIG. 30B). Notably, linsitinib treatment at 12 µM of the cancer cell monolayers over 72 hours showed significant drug efficacy against both the non-metastatic and metastatic cell lines, an observation at odds with clinical data (FIG. 30C). These samples were also analyzed using an ELISA to verify linsitinib mechanism of action - significant decreases of levels of phosphorylated IGF-1R (FIG. 30D).

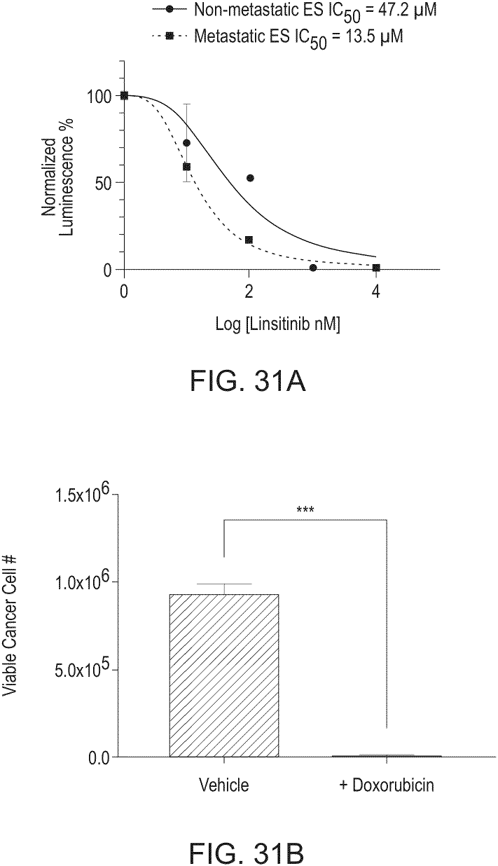

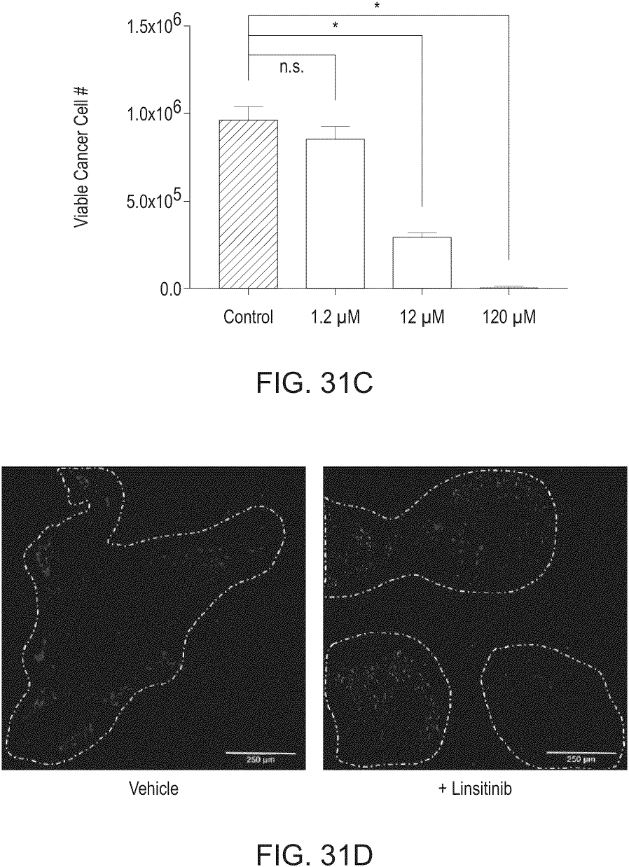

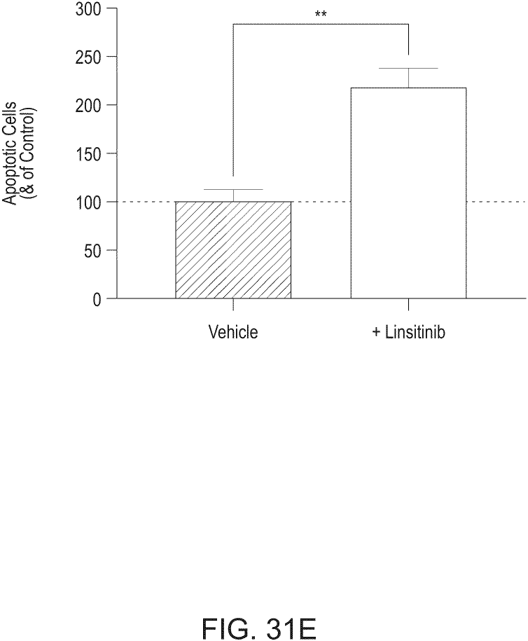

Having determined that luminescence of the transduced cancer cells could serve as a reliable indicator of ES cell viability in monolayers, we next verified that this method can be used for the TE-ES models, by exposing the non-metastatic TE-ES to 1 µM of doxorubicin for 72 hours (FIGS. 31A and 31B).

FIGS. 31A-E show aspects of the evaluation of the dose-dependent effects of doxorubicin and linsitinib on engineered non-metastatic and metastatic ES bone tumors. FIG. 31A shows the effects when non-metastatic and metastatic ES cell lines were treated with various concentrations of doxorubicin (0, 10, 100, 1,000, and 10,000 nM) over 72 hours. An MTT viability assay was used to determine drug toxicity, with luminescence generated by viable cells measured at the endpoint (10⁴ cells per well, mean± s.e.m., n = 3). FIG. 31B shows the effects when non-metastatic engineered ES bone tumors were treated with 1 µM doxorubicin for 72 hours and viability was determined by measuring luminescence of these cells (mean ± s.e.m., n = 3). FIG. 31C shows the effects when non-metastatic engineered ES bone tumors were treated using a range of concentrations of linsitinib (0, 1.2, 12, and 120 µM) for 72 hours and viability was determined by measuring luminescence of these cells (mean ± s.e.m., n = 3). FIG. 31D shows the TUNEL analysis for apoptosis of untreated (left) and 12 µM linsitinib treated (right) non-metastatic engineered ES bone tumors. Tumors are outlined by white dashes and validated using corresponding H&E stains of the identical loci. All scale bars: 250 µm. FIG. 31A shows the effects when TUNEL Cell Counter software was used to analyze the TUNEL stain images to determine absolute numbers of apoptotic cells per representative image (mean ± s.e.m., n = 4). *P < 0.05; **P < 0.01; ^{∗∗∗}P < 0.001 by two-way ANOVA with Bonferroni post-test or unpaired two-tailed Student’s t test.

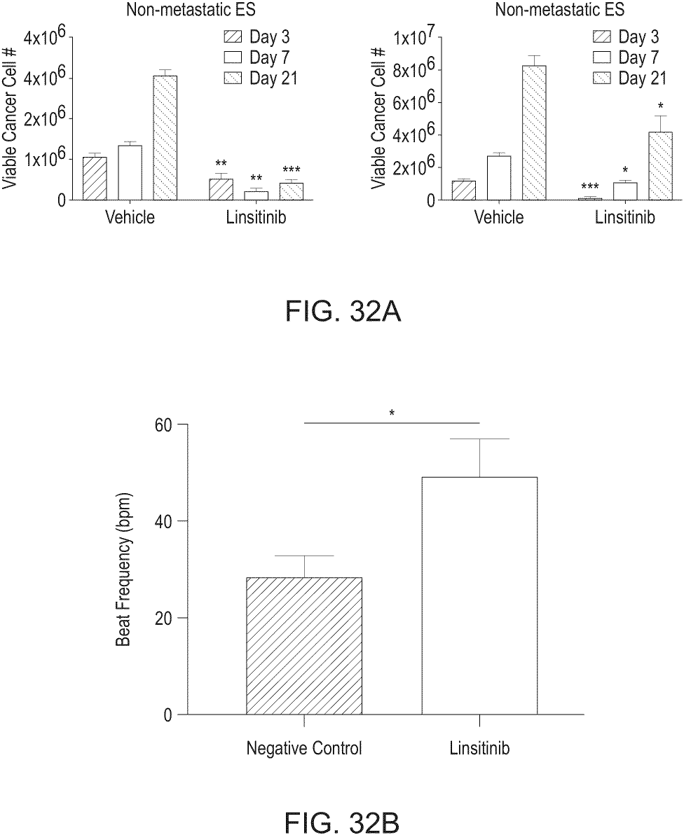

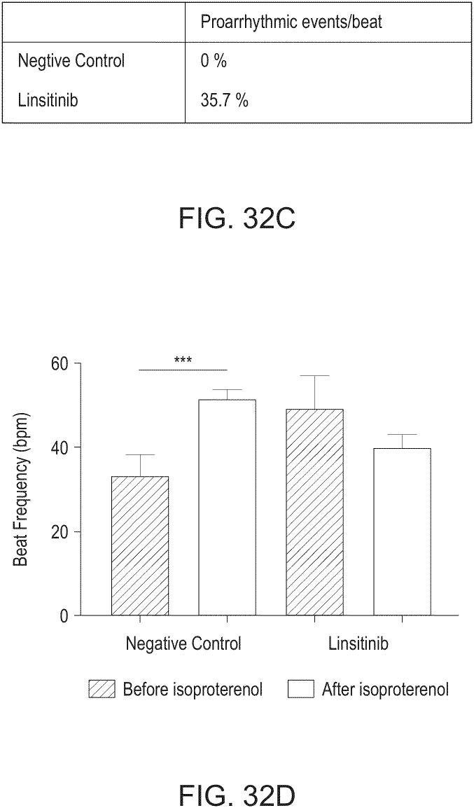

FIGS. 32A-D show aspects of responses of human engineered bone ES tumors and cardiac tissues to linsitinib in isolated platform chambers. FIG. 32A shows responses when non-metastatic (left) and metastatic (right) ES tumors were exposed to linsitinib (12 µM) according to the 3-week drug treatment regimen used in a Phase II clinical study. Luminescence as a function of cancer cell number and viability was measured (mean± s.e.m., n = 6 for day 3, and n = 3 for day 7 and 21). FIG. 32B shows beat frequency of cardiac tissues after exposure to linsitinib (12 µM) (mean± s.e.m., n = 11). FIG. 32C shows the occurrence of proarrhythmic events/beat after exposure to linsitinib. FIG. 32D shows beat frequency of human cardiac tissues exposed to linsitinib after isoproterenol exposure (mean ± s.e.m., n = 6-9). *P < 0.05; **P < 0.01; ***P < 0.001 by two-way ANOVA with Bonferroni post-test or unpaired two-tailed Student’s t test.

The effects of linsitinib were studied in an experiment performed according to the 3-week treatment cycle used in the clinical trial (3 days of drug administration followed by 4 days without the drug, in 3 cycles), with luminescence signal serving as an indicator of cancer cell viability within the TE-ES. A dose-dependent response was observed for the non-metastatic TE-ES model, with significant reduction in cell viability at linsinitib concentration of 12 µM (Fig). TUNEL assay showed increases in apoptosis, corroborating the luminescence viability findings (Fig).

In order to better understand the linsitinib response for metastatic and non-metastatic tumors, luminescence signals were measured following 3, 7, and 21 days of treatment. Already after 3 days, a significant drug response was observed in both TE-ES tumor models, just as it has been observed in monolayers of cancer cells (FIG. 32A, FIG. 30C). However, there was a difference between the non-metastatic and metastatic TE-ES model responses across the entire 21-day clinical drug treatment regimen, which could not be observed in monolayers due to extensive cell proliferation. Linsitinib caused an initial decrease in cancer cell population in the non-metastatic model and this population appeared unable to continue expanding. In contrast, after a significant response to the drug after 3 days, the metastatic model displayed a decrease in drug efficacy, as the cancer cell population continued to expand over the 21-day treatment cycle (FIG. 32A). Unlike the corresponding monolayer results, this observation is in line with the clinical results for metastatic ES - poor outcomes despite aggressive chemotherapy.

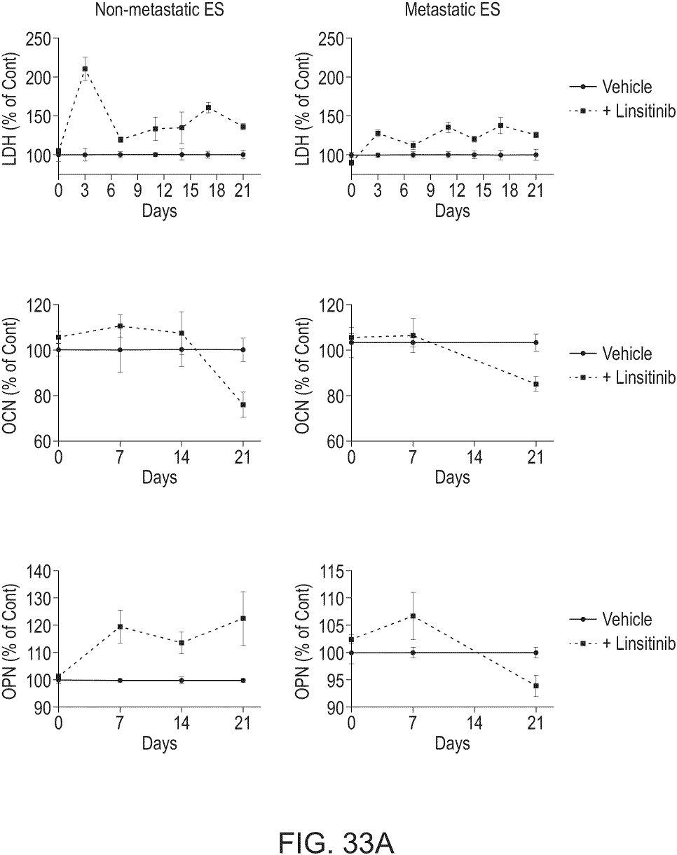

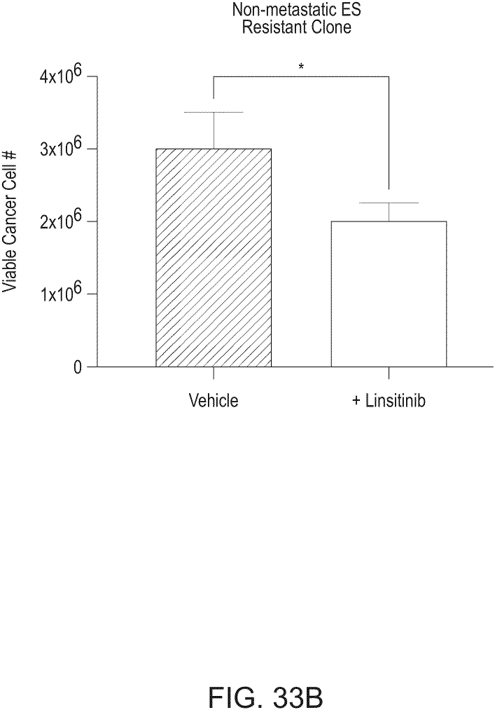

Determining the effects of linsitinib on ES cells and osteoblasts within the engineered ES bone tumors across a 3-week clinical drug treatment regimen is shown in FIGS. 33A-B. Supernatants collected at regular intervals were analyzed for cytotoxicity and secreted proteins indicating the role of osteoblasts in responses to the linsitinib treatment regimen. FIG. 33A shows effects when non-metastatic (left) and metastatic (right) engineered ES bone tumors were exposed to linsitinib (12 µM) according to the 3-week drug treatment regimen used in a clinical study we aimed to recapitulate. Supernatants were collected from each individual sample across the duration of the drug treatments and were analyzed for osteoblast function (OCN, OPN) and for cytotoxicity (LDH) (mean± s.e.m., n = 3). Linsitinib resistant cancer cells from the responsive, non-metastatic ES tumor tissues were sorted following one 3-week round of the 12 µM treatment regimen. Sorted cells were then used to create new engineered bone tumors that were exposed to another round of 12 µM linsitinib treatment regimen. Luminescence as a function of cancer cell number and viability was measured (mean± s.e.m., n= 3, day 21, biologically independent per group). *P < 0.05 by unpaired, two-tailed Student’s t-test (FIG. 33B).

Lactic acid dehydrogenase (LDH) secretion indicated that cytotoxicity spiked in both models immediately following drug administration, but significantly more so in the responsive, non-metastatic ES model (FIG. 33A). Osteocalcin secretion decreased after drug treatment in both models, suggesting suppressed osteoblast function (FIG. 33A). Interestingly, osteopontin, known to play a stabilizing role for cancer cells in bone niches, was significantly increased throughout the 21 days in the non-metastatic, linsitinib-responsive ES model, while it decreased similar to osteocalcin in the metastatic, non-responsive ES model (FIG. 33A). Given the positive response to linsitinib observed in the non-metastatic ES tumor model, we next isolated the drug resistant cells by sorting, expanded this subpopulation and used it to generate new tumor models. These tumors were subjected to another 21-day treatment regimen, to try to further assess the lack of their response to linsitinib. Interestingly, these ES resistant-cell derived tumors again showed a significant initial drug response (FIG. 33B), in line with the hypothesized transient, insulin receptor dependent resistance, as opposed to the “inherited” pathway for IGF1-R inhibitor resistance.

### Responses to Linsitinib of Engineered Cardiac Tissues Cultured in Isolation

After establishing the capability of TE-ES tumors to model drug efficacy, we evaluated the ability of cardiac tissues to determine the cardiotoxicity of linsitinib. Cardiac tissues were exposed to the same therapeutic concentration of linsitinib as bone tumors. The cardiac model responded with increased beating frequency after 3 days of exposure to the drug. Cardiotoxicity of linsitinib has been observed in clinical trials of other types of cancer, with patients presenting proarrhythmic events, like tachycardia (3.75-5 % of patients) and atrial fibrillation (3.75 - 5 %). We observed higher beat frequency and higher rate of proarrhythmic events per beat (around 36%) than in clinical studies (FIGS. 32B and 32C). When the cardiac tissues exposed to linsitinib were subsequently exposed to isoproterenol, the inotropic response was not observed, suggesting lasting effects (FIG. 32D).

Overall, when bioengineered cardiac tissues were exposed to linsitinib in an isolated setting, we observed induction of tachycardia, proarrhythmic events, and altered physiological responses to isoproterenol. The occurrence of proarrhythmic events at a rate higher than seen clinically, and the increased sensitivity observed for beat frequency and isoproterenol response suggest that this model on its own fails to predict clinical responses. The same can be said for the non-metastatic TE-ES model, which showed significant drug response for the duration of the 3 weeks drug treatment regimen despite the lack of success in the Phase II clinical trial.

### Responses to Linsitinib of the Bone ES Tumor and Cardiac Tissue in the Integrated Platform

In patients, bone tumor and cardiac tissue do not exist in isolation, and are not necessarily exposed to the same drug concentrations. Tissue-tissue communication would further increase the physiological relevance of these models. Towards this goal, and in order to demonstrate that an integrated model (with the tumor and cardiac tissues connected by microfluidic perfusion) is more physiologically relevant for predictive drug screening, we studied the effects of linsitinib on the heart and bone tumor tissues simultaneously cultured and exposed to the drug in the integrated platform.

First, we determined the effects of the combined culture medium (1:1 mixture of bone tumor and cardiac media in the platform) on each engineered tissue. Importantly, the base media for both tissues are identical, except for one supplement (fetal bovine serum or B-27™) To this end, we cultured the non-metastatic TE-ES tumor (which responded to linsitinib treatment and therefore deviated from the clinically relevant observations) in bone tumor media (isolated culture), 1:1 mixed media (integrated platform), and in cardiac media (as a control) for the duration of the clinical drug treatment regimen (3 weeks).

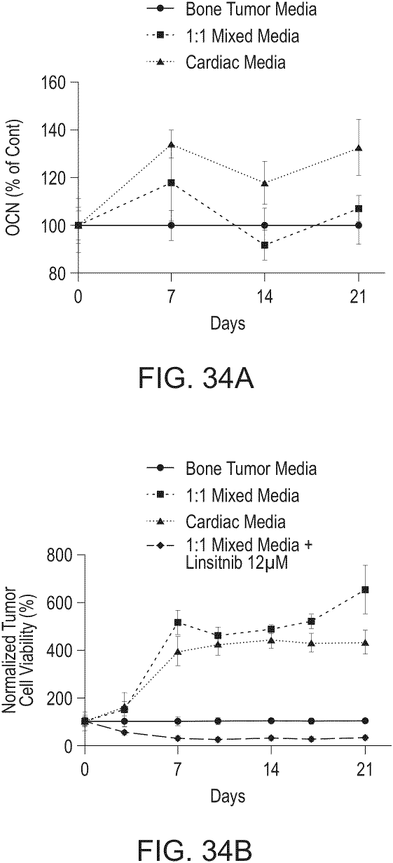

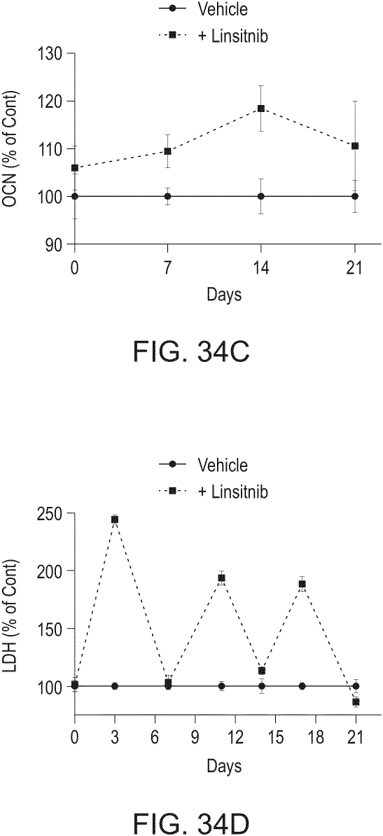

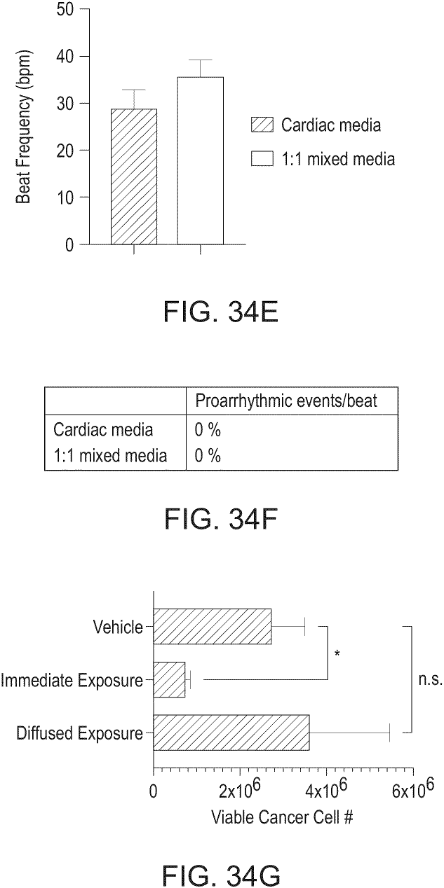

FIGS. 34A-G summarize the evaluation of the effects of linsitinib on the non-metastatic engineered ES bone tumor and cardiac tissues in the integrated platform. Engineered non-metastatic ES bone tumors were grown in bone tumor media (isolated culture conditions), 1:1 mixed media (integrated platform conditions), or cardiac tissue media for 21 days. Supernatants collected every 7 days were then analyzed for secretion of OCN (osteoblast marker) (mean ± s.e.m., n = 3). Samples were also analyzed every 3 days for luminescence values as a function of viable cancer cell number (FIGS. 34A and 34B). Engineered non-metastatic ES bone tumors grown under the integrated platform conditions (1:1 mixed media) were treated with linsitinib (12 µM) according to the previously outlined 21-day clinical treatment regimen. Luminescence (FIG. 34B), OCN (Fig), and LDH secretion (FIG. 34D) as functions of cancer cell number and viability as well as osteoblast function and cytotoxicity, respectively, were measured (mean ± s.e.m., n=3). Heart tissues were grown in either standard conditions (cardiac media) or under the integrated platform conditions (1:1 mixed media) and their beat frequency measured across 72 hours (mean ± s.e.m., n=4-11) (FIG. 34E). FIG. 34F shows the occurrence of proarrhythmic events after the heart tissues being cultured under cardiac media or 1:1 mixed media (mean ± s.e.m., n=?). Engineered non-metastatic ES bone tumors and cardiac tissues were exposed to linsitinib (12 µM) over a period of 72 hours in the perfused integrated platform. Linsitinib was either introduced into the reservoir and allowed to circulate and diffuse into the tissues under physiologically relevant fluid flow conditions (diffused exposure) or introduced directly into the tissue chambers (immediate exposure). Luminescence as a function of cancer cell number and viability was measured (mean ± s.e.m., n = 6 for day 3, and n = 3 for day 7 and 21). *P < 0.05; **P < 0.01; ***P < 0.001 by two-way ANOVA with Bonferroni post-test or unpaired two-tailed Student’s t test. (FIG. 34G).

No significant differences were observed in the osteoblast bone niche, and the osteocalcin levels were also similar for the bone tumor media and the mixed media (FIG. 34A). Longitudinal luminescence readouts used to track ES cells showed faster growth in the 1:1 mixed media and cardiac media, suggesting that the B-27™ supplement could be contributing to the increased proliferation (FIG. 34B).

The TE-ES models with mixed media were subjected to the same 12 µM linsitinib treatment regimen as the isolated cultures. Luminescence readings of cancer cell viability within the engineered tissues showed that despite significant increases in cancer cell proliferation in the mixed media, the drug was still effective at killing cancer cells and maintaining their population at a significantly lower level (~30 % of their starting population) (FIG. 34B). Meanwhile OCN secretion increased only slightly, while similar peaks in LDH secretion (indicating cytotoxicity) were noted immediately following the 3 days of drug exposure at days 3, 11, and 17, as for the bone tumor media (FIGS. 34C and 34D). Therefore, while some differences in cancer cell proliferation were noted in the mixed media, the responses to linsitinib were comparable.

Engineered cardiac tissues in mixed media showed no change in beat frequency (FIG. 34E) or proarrhythmic events (FIG. 34F) relatively to tissues in cardiac media.

The TE-ES and cardiac tissues were then cultured in the integrated platform with perfusion of mixed media. Linsitinib was introduced into the reservoir and delivered to tissues via circulation of perfusate and diffusion into the tissues.

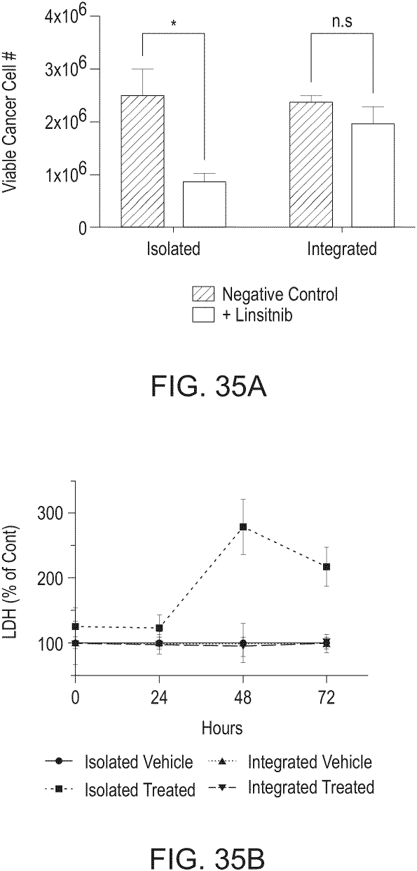

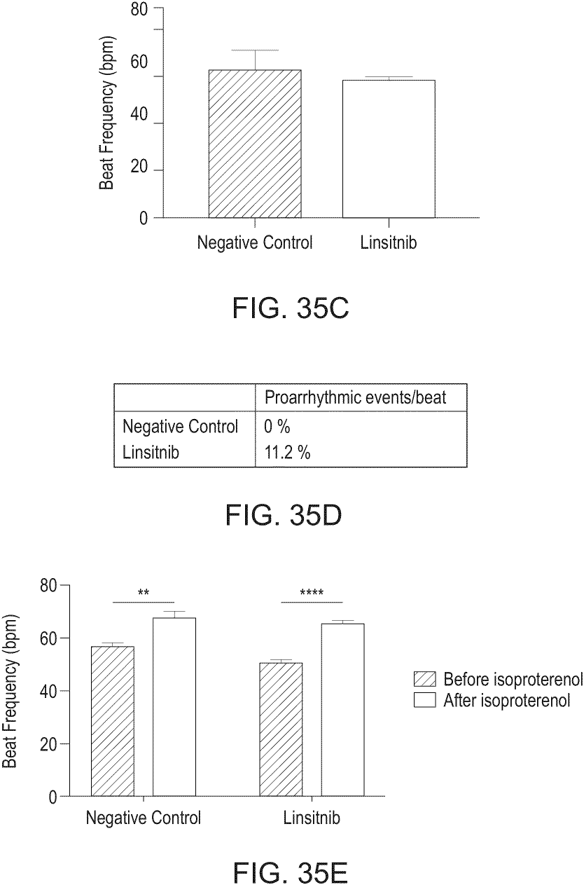

FIGS. 35A-E show aspects of responses of human engineered bone ES tumors and cardiac tissues to linsitinib in the integrated platform with microfluidic perfusion. FIGS. 35A and 35B show the responses when non-metastatic ES bone tumors and cardiac tissues were exposed to linsitinib (12 µM) over a period of 72 hours in either isolated culture or within the perfused integrated platform. Luminescence (FIG. 35A) and LDH secretion (FIG. 35B) as functions of cancer cell number and viability as well as cytotoxicity, respectively, were measured (mean± s.e.m., n = 3). FIG. 35C shows beat frequency of cardiac tissues after exposure to linsitinib (12 µM) within the perfused integrated platform (mean ± s.e.m., n = 3). FIG. 35D shows the occurrence of proarrhythmic events/beat after exposure to linsitinib within the platform. FIG. 35E shows beat frequency of cardiac tissues that had been exposed to linsitinib in the platform after isoproterenol exposure. *P < 0.05; **P < 0.01; ***P < 0.001 by two-way ANOVA with Bonferroni post-test or unpaired two-tailed Student’s t test.

Following 3 days of treatment, luminescence signals from the engineered non-metastatic ES bone tumor tissues revealed insignificant drug response (as observed in clinical studies) and in contrast to both the monolayer cell cultures and isolated TE-ES culture (FIG. 35A). Secretion of LDH, a cytotoxicity marker, showed no significant difference between the vehicle and linsitinib treated samples (FIG. 35B), in agreement with the luminescence viability data.

ES cells, when co-cultured with mesenchymal stem cells and exposed to physiological shear-stress in a perfusion bioreactor, can become resistant to IGF-1R inhibitors. Therefore, we evaluated the role of flow derived shear stress in this newly found resistance of non-metastatic TE-ES bone tumor tissues to the IGF-1R inhibitor linsitinib. Linsitinib was introduced into the platform (12 µM, 3 days), either via circulation or directly into the TE-ES tissue chamber (FIG. 34G), to distinguish effects of flow-derived stimuli from drug diffusion into the tissues. Immediately exposure to the drug resulted in fast response to the drug akin to that observed in isolated cultures. In contrast, introduction of linsitinib into the circulation showed no response. These results agree with observations from the clinical trial, since linsitinib was unable to stop progression of ES, with no patients completing the trial, and suggest that the integrated model may better mimic the clinical scenario than the traditional models.

In the cardiac tissue model, we did not observe linsitinib-mediated changes in beat frequency, suggesting that the occurrence of false responses was reduced (FIG. 35C). Similarly, the rate of proarrhythmic events in the integrated model (approximately 11%) was much closer to the rates observed clinically (FIG. 13D). When the cardiac tissues exposed to linsitinib were subsequently exposed to isoproterenol, we observed the expected inotropic response (FIG. 35E).

Overall, in the integrated platform, linsitinib induced no change in beat frequency, was associated with a rate of proarrhythmic events similar to clinical data, and retained the physiologically healthy response to isoproterenol, suggesting mild cardiotoxicity.

The potential of the platform we present here is in their ability to better agree with clinical results than the traditional preclinical models. The integrated platform contained the Ewing sarcoma tumor (formed by introducing primary cancer cells into the engineered human bone) and the engineered human cardiac muscle (formed by electromechanical conditioning of iPS-derived cardiomyocytes and supporting fibroblasts in fibrin gel), connected by microfluidic circulation. This platform recapitulated unexpected results of a Phase II clinical trial of linsitinib, a small-molecule tyrosine kinase inhibitor of the insulin-like growth factor receptor (IGF-1R).

The platform design allowed real-time in situ monitoring of cancer cell growth and simultaneous assessment of the drug efficacy and cardiotoxicity. The platform’s flexibility and ease of use allow the design to be tailored to the questions being asked.

Also, the use of polysulfone as the main device fabrication material (instead of the widely utilized PDMS) avoids uncontrollable absorption of hydrophobic compounds, which most chemotherapeutics are. The open setting also allows for imaging and sampling of tissues and culture media.

Referring back to FIG. 2G an example of the modularity of the multi-organ platform is shown. Complexity of the platform is user-determined, from a single to multiple serial connected platforms (in this example, 16 tissues serial connection), for scaling of the system.

The biological fidelity of the engineered tumor and heart tissues was documented by a battery of the known responses to standard drugs. We also demonstrated clear advantages of engineered tissues over the monolayer culture, and of the tissues connected by microfluidic circulation over isolated tissue culture in recapitulating clinical data.

### EXPERIMENTAL DESIGN

Breast tumor biopsies are digested with collagenase, and isolated BC cells are embedded within Cultrex® basement membrane matrix for expansion as organoids in 3D culture. Organoids made using these protocols have demonstrated a high degree of biological fidelity to their corresponding native patient tumors. Followingly, we have built an in-house panel of HR+ and TNBC patient-derived organoids.

Morphologically, a majority of BC organoids matched the histopathology and hormone receptor status of their donors. The patients’ genetic diversity was also largely captured by these organoids, which recapitulated key copy number alterations, mutational load and signatures, and mutations in well-established cancer driver genes. When gene expression profiles of BC organoids were compared to those of >1,100 BC from The Cancer Genome Atlas (TCGA), they clustered with the corresponding subtypes. Importantly, it was shown that the BC organoids responded to drug screening in predictable ways, namely with HER2+ organoids being significantly more sensitive to drugs blocking the HER signaling pathway and organoids with high BRCA½ signatures being more responsive to Poly(ADP-ribose)polymerase inhibitors (PARPis) that haven proven successful in patients with such a defective pathway. Missing thus far is a comprehensive comparative gene expression and clonal development analysis between the organoids and matched patient samples. To generate BC organoids for such analysis, we can continue to rely on biopsies of primary and matched metastatic sites of chemo-naive patients (metastatic at diagnosis), with focus on HR+ tumors (that metastasize to bone, liver, lung), and TNBC tumors that prevalently metastasize to lung. As controls, we can analyze patient-matched normal breast tissue.

Validation Engineered tumors can be validated against native tumors via scRNAseq, mutational and exome profiling, IHC, metabolic, and secretome analyses.

Anticipated results, potential difficulties, and alternate approaches: We can use native tumor benchmarks to show that engineered tumors recapitulate key hallmarks of metastatic progression, including expression of proliferative and metastatic markers. We expect that breast tumor progression can increase as a function of matrix stiffness and angiogenesis. Given our previous success with these models, challenges may be limited to isolation of well-defined cell populations from primary tumors. Rigorous characterization of these cells can be performed to understand any aberrant behavior of the bioengineered models due to phenotypic heterogeneity. If extended tumor culture (>4 weeks) poses challenges, we can adjust perfusion conditions to improve long-term control of nutrients, oxygen and regulatory factors.

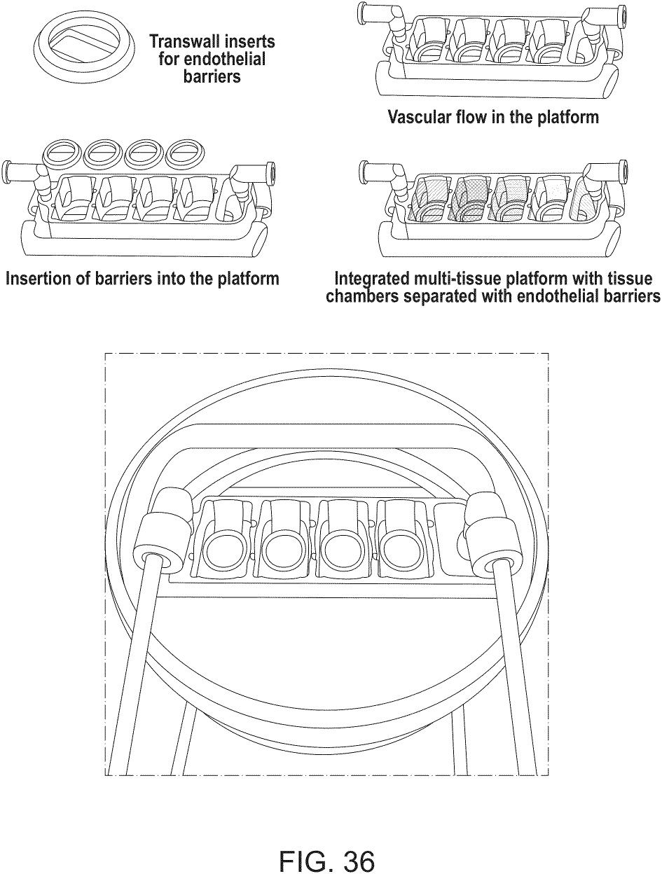

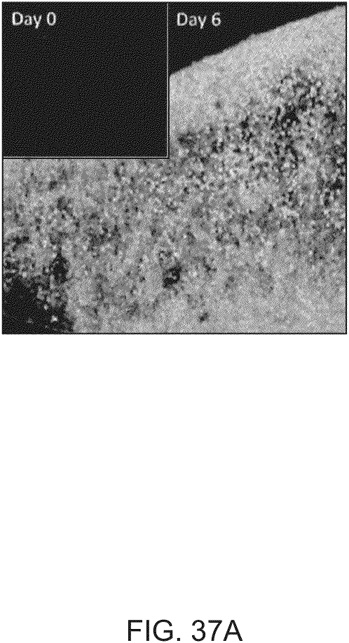

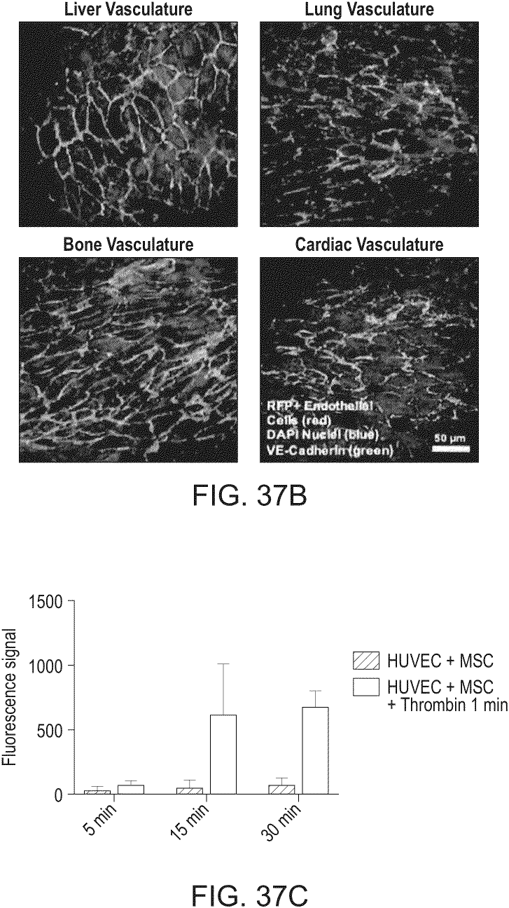

Vascularization The generation of a patient-specific vascular barrier is critical for functionally connecting tissue compartments while maintaining each tissues, as well as vascular perfusion, under optimal culture conditions resulting in phenotypic stability and maturation, analogous to the separation of interstitial and intravascular compartments in vivo. Transwell mesh inserts in each tissue chamber were coated with fibronectin and seeded with ECs and supporting MSCs with 2:1 ratio (FIG. 36). A stable endothelial barrier was then established by exposure to flow shear (ramped up from 1 to 5 dyn/cm² over 5 days of culture). To physiologically integrate engineered tumors and host tissues by vascular flow, the endothelialized membrane needs to separate the interstitial and intravascular spaces, enabling vasculature-mediated tissues crosstalk. These conditions resulted in a physiologically low permeability of the endothelial barrier to both small and large regulatory molecules (3 and 70 kDa FITC-labelled dextrans) and in a physiologic increase in barrier permeability in response to thrombin (FIGS. 37A-C). Such response to an inflammatory agent is critical for metastatic extravasation of cancer cells. The endothelial barrier allowed optimization of culture conditions for each tissue - akin to compartmentalization in the body that avoids the use of “common” media. This approach promoted long term culture (4-12 weeks) of matured tissues, with on-line readouts (cell tracking, viability, function) in longitudinal and dynamic studies.

FIG. 36 shows a configurable platform in which the tissues are connected by vascular perfusion with endothelial barrier and circulating cancer and immune cells.

FIGS. 37A-37C shows aspects of bioengineering of stable and tissue-specific endothelium with tunable permeability. The endothelial barrier was engineered by coating the membrane insets by supporting MSCs and iPS-ECs, under flow of vascular medium (5 dyn/cm²). Over time in culture, the endothelium started to acquire tissue-specific properties (FIG. 37A). The physiologically low vascular permeability increased by exposure to thrombin and other inflammatory agents. In all tissue compartments, the endothelium maintained a barrier between tissue and vascular flow (FIG. 37B). Of note, macrophages (fluorescently labeled) show ability to penetrate and invade cryoinjured cardiac tissue through the endothelial barrier (FIG. 37C).

To physiologically integrate circulating metastatic BC cells and host tissues by vascular flow (first using primary cells and BC cell lines, then matched patient-derived BC cells and iPSC-derived tissues), they can be connected in modular format by placement into chambers above the endothelialized membrane that separates tissue and vascular media, thus enabling vasculature-mediated tissue crosstalk (FIGS. 1 and 36). Bioluminescent and fluorescent BC cells can be introduced into circulation, and their metastatic progression across the 4 host tissues can be tracked (bioluminescence to track cell motion and proliferation; fluorescence to monitor cell colonization and seeding). BC cells can then be re-isolated from both circulation and from tissues positive for extravasation using flow cytometry cell sorting and analyzed using scRNAseq (see Aim 3). These methods can be tested across multiple BC cells and matching iPSC-generated tissues to capture patient specific effects.

Modeling extravasation and survival of circulating BC tumor cells (CTC): Cancer progression and treatment depend on the tumor cell ability to migrate to distant sites, and on drug transport from vascular circulation to these sites. Blood vessels have dynamic and heterogeneous barriers that select which entities can pass through. Vascular permeability is regulated by the EC lining of the vessels, serving as a gatekeeper for the exchange of cells and molecules between tissue and blood, and it also influences the maximum achievable drug concentration in primary and secondary tumors. Previous studies have shown that tumor cells increase endothelial permeability, suggesting its role in intra/extravasation. By tracking labeled CTCs, we can be able to study the mechanistic role of the vascular barrier in metastasis. We can initially use HR+ and TNBC cell lines labeled with fluorescent and bioluminescent tags and infused into the circulation to study their extravasation through the endothelial barrier and seeding into host tissues. Important variables can be BC cell line used, the host tissue type, order of vascular flow through the platform, and endothelial permeability.

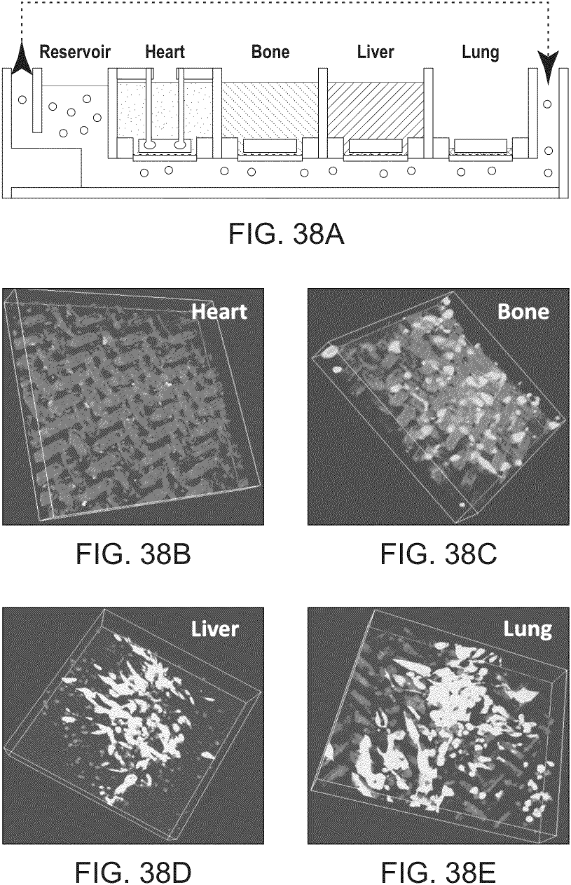

Preliminary studies support the critical role of the endothelial barrier for selective metastatic homing of circulating TNBC cells. In its absence, luminescent TN MDA-MB231 BC cells non-selectively colonized all host tissues, and at a pace that is physiologically irrelevant (data not shown), demonstrating the importance of a physiological vascular barrier to ensure host tissue maturity and faithfully recapitulate metastatic extravasation and homing of tumor cells into target tissues. Moreover, we showed that homing to specific host tissues relies on more than just tissue specific media and tissue-derived ECMs, as this leads to non-specific accumulation in all tissues including the negative control heart tissue (data not shown). In fact, we only observed physiologically relevant metastatic progression when using a combination of a functional vascular barrier and mature primary cell derived tissues within our integrated perfused platform, suggesting that secretion of target-specific chemo-attractants (chemokines, cytokines, extracellular vesicles) is critical for this process (FIGS. 38A-E). After introducing GFP-labelled MDA MB231 cells into circulation, we were able to capture using confocal microscopy the various stages of metastatic progression: (i) successful circulation of only a small fraction of injected cells in the platform, (ii) arrest along the vascular wall of the specific host tissue, (iii) extravasation through the EC barrier and (iv) seeding and colonization of the host tissue. Of note, we only observed extravasation and tissue seeding in the typical target tissues (lung, liver, bone) but not in our negative control (cardiac) (FIGS. 38A-E).

FIGS. 38A-E show selective extravasation of breast cancer cells into the tissue compartments. GFP-expressing TNBC cell line MDA-MB231 introduced into circulation selectively extravasated into the bone, liver and lung tissues, but not into the heart tissue.

Proliferation of metastasized breast cancer cell lines within their target tissues: Based on our preliminary results, we hypothesize that continued culture would allow for growth of the surviving metastasized cells to an extent amenable to their further investigation, including by re-isolation, scRNAseq, and subsequent drug targeting . We can establish luminescence-based online readout of metastasized BC cell populations, based on methods already in use for in vivo mouse xenotransplants. We can also vary the cellular complexity of engineered host tissues and assess which stromal environments favor metastatic cell homing, by evaluating a variety of stromal cells (tissue-specific endothelium, fibroblasts, and stroma; tissue-resident macrophages). We have previously demonstrated that supporting cells, including EC and MSC, mediate metastatic homing of breast cancer cells into bone, and can thus characterize the effects of angiogenic factors (VEGF, FGF, EGF) on metastatic cell homing. Given our established functional cardiac tissue, we can investigate why cardiac tissues may not support metastatic growth (e.g., due to the heart beating or unique secretome signature), with possible new insights that may have therapeutic potential. Also, we have recently introduced into our multi-tissue platform the circulating immune cells (monocytes, macrophages), and showed their ability to penetrate through endothelial barriers into the tissues (e.g., following cryo-injury or inflammation, FIGS. 37A-C), allowing us to study the predicted role of some immune populations in facilitating or preventing metastasis.

Recapitulation in vitro of the targeted BC metastasis already observed in vivo: From a bioengineering validation perspective, a major weakness of the TN MDA-MB231 BC cell line is its ability to disseminate non-specifically, across a wide variety of tissue models (liver, lung, bone, brain). We can thus also rely on tissue-specific metastatic TNBC cell lines that have been shown to selectively target either the lung or the bone, when injected into tail veins of mouse xenotransplants. Bioluminescent lung targeting LM2 and bone targeting BoM-1833 variants of the MDA-MB231 cell line, kindly provided to us by the pioneering J. Massague lab through an MTA, can be introduced into circulation within our integrated tissue platform and their dissemination to the appropriate target tissues can be monitored. Extravasated LM2 and BoM-1833 cells can be sequenced for gene expression and compared to the results found in vivo. Additionally, we can use the approach used by the Massague lab in mouse models to establish tissue specific cell sub-lines but within the in vitro human tissue context. Therefore, we can isolate the extravasated MDA-MB231 cells from lung and bone tissues and re-introduce them into circulation to determine whether they have gained target specificity (i.e. if those isolated from bone would preferentially target bone). Gene expression profiling can be performed on these extravasated cells to determine phenotypic changes linked to tissue specific metastasis, such as to genes like ADAMTS1, FGF5, FST, PRG, CTGF, IL11, MMP1 and CXCR4 that have been shown to govern bone extravasation.

Generation of the “patient on a chip” in vitro model of BC metastasis: We have already demonstrated in our previous and preliminary studies the capability for generating a broad range of human tissues from iPSCs and for modeling metastatic progression in vitro using cell lines. A patient-specific platform, with tunable mechanical and biochemical host tissue properties, can allow investigation of preferential metastasis of fluorescently labeled patient tumor cells into lung, liver or bone, but not into the heart. Boyden chamber style transendothelial migration assays can be initially be used to screen the patient cells for their intravasation and extravasation potential prior to both their introduction to the multi-tissue platform and to the development and integration of iPSC-derived host tissues. For intravasation studies, we can culture the patient-derived BC organoids in a basement membrane based 3D tumor model that can be integrated by vascular perfusion to the engineered host tissues. Intravasation of tumor cells can be monitored by tracking fluorescently labeled tumor cells, and the extent of intravasating tumor cells and their lifetime in circulation can be assessed. The potential extravasation of these cells in circulation into the target tissue chambers can then be monitored and quantified for different patients of HR+ or TNBC breast tumor subtypes.

We can further manipulate physicochemical properties of the host tissues (for example ECM stiffness) to mimic organ conditions in high-risk patients (age, inflammation), and study their effects on CTC seeding efficiency and specificity. The cells that invade the host tissues can be characterized by single-cell RNA sequencing, to determine their phenotypes associated with metastatic seeding into the specific host tissues.

Investigating the cellular subpopulation most likely to metastasize, via track-labeling and scRNASeq profiling in the engineered platform, can enable characterization of “pro-metastatic” cell phenotypes within the heterogeneous populations derived from the tumor and perhaps across the heterogeneous patients. These data can serve as input for elucidation of novel targets and pathways, and for predicting therapeutic treatment options. The pathology of host tissues colonized with tumor cells can be characterized by histology/IHC staining, and compared with clinical specimens. We can also conduct secretome analyses for tumor-specific metastasis-driving EVs before and after the organoid-grown tumor cells are introduced into the circulation, and before and after homing, to understand what signaling events could underlie the metastatic phenomena. To validate the model, we can benchmark metastasis in the platform with the metastatic bone tumors from patients, and evaluate the tissue specificity and colonization potential of metastatic cells.

To characterize the biological relevance of bioengineered tumors, tissue from both primary and metastatic site can be dissociated, exome/RNA-Sequenced, and compared to patient-matched surgical samples. To demonstrate the utility of the “cancer patient on a chip” for systems biology studies, we can experimentally test OncoTreat-predicted drug responses as inferred by meta VIPER.

Large-scale single-cell analysis for tumor model validation, tracking metastatic populations and inferring drug responses. Throughout the proposed studies, we can take advantage of recent advances in large-scale scRNASeq for three main purposes: (a) Validation and study of primary tumor model; (b) Analysis of metastases in host tissues (bone, liver, lung); and (c) Inferring single-cell-specific drug sensitivity for primaries, CTCs, and metastases by OncoTreat analysis. We have established a computer-controlled microfluidic platform for scRNASeq that can parallel-profile ~10,000 cells with rapid, high-efficiency cell capture (>50%), using optical microscopy for on-chip analysis of markers, cell viability, and cell lysis.

The device has a simple flow cell with an array of insertable devices, i.e., microwells on a glass slide. Typically, one cell per microwell is gravity-loaded, followed by loading of polymer beads coated with oligonucleotide primers that contain a universal adapter sequence, a bead-specific barcode sequence, a unique molecular identifier (UMI), and oligo(dT) for mRNA capture. Cell lysis and reverse transcription occur automatically, following introduction of strongly denaturing lysis buffer and a perfluorinated oil to seal the array. On-chip fluorescence imaging facilitates control of cell viability, lysis, sealing, and counting. Following mRNA capture, we introduce a detergent-containing buffer to rapidly remove sealant and lysate, such that barcoded beads with hybridized mRNA are exposed, allowing reverse transcription to occurs automatically. cDNA-coated beads are pooled, harvested, and 3′-end RNA-Seq libraries are generated using the SCRB-Seq protocol and the Nextera transposition system (Illumina), at a cost as low as $0.05/cell.

We sequence the pooled libraries with paired-end sequencing, where the first read contains both the cell-identifying barcode and a unique molecular identifier (UMI) barcode, and the second read contains transcript sequences. For the proposed studies, we can align read two to the human genome using the STAR aligner with a transcriptome annotation, to take advantage of its splice-awareness. By combining the alignment with the forming barcode in the first read, we assign an address to each read that aligns uniquely to a locus that can be unambiguously assigned to an annotated gene. Given the strand-specific nature of the libraries, strand information can be used to ensure proper alignment. The address includes the read ID, cell-identifying barcode, UMI, and gene symbol. This algorithm gives us an estimate of the number of captured mRNA molecules associated with each gene in each cell.

We have also developed a computational pipeline for scRNASeq data analysis that includes unsupervised clustering, data visualization, and differential expression. “Drop-out” events, i.e., genes present in a given cell may be undetected, are exploited to identify markers of cell subpopulations, because genes detected in fewer cells than expected are likely markers of specific cell subpopulations. Using “drop-out” analysis for unsupervised identification of markers, we can generate a correlation matrix that is then used to identify clusters of cells with similar patterns of gene expression. These clusters can be visualized using various methods, including t-stochastic neighborhood embedding (t-SNE), UMAP, and diffusion component analysis.

Once single-cell clustering is completed, we can conduct differential expression and meta VIPER analysis to identify cross-compartment cellular subpopulations with conserved gene expression or protein activity signatures. While gene expression clustering is useful to identify molecularly distinct subpopulations, it is less adept at capturing quantitative gene expression differences between cells. For this, we can employ the SCDE algorithm that models the over-dispersion in scRNASeq counting data with the negative binomial distribution, as in bulk RNA-Seq, but also integrates a Bayesian probabilistic treatment of transcript drop-out. MR dependencies can be validated by pooled, barcoded CRISPR/Cas9 silencing of top candidate MRs in the population of interest, and by measuring depletion of barcoded cells in the progressed populations.

Sample preparation can be technically challenging, due to incomplete dissociation, low viability, and low-quality RNA. Our microfluidic system allows diagnosis of dissociation quality and viability on-the-fly. However, issues with a sample type or component are encountered, single-nucleus RNA-Seq is an alternative approach compatible with the device, which accommodates the lysis conditions required to disrupt nuclei, and circumvents.

### METHODS

### Integrated System

The main manifold of the platform was machined using a 3-axis computer numerical control (CNC) milling machine from polysulfone and incorporated reservoirs for individual tissues and an additional reservoir and fluidic ports for circulating media. The connection channel was defined by a recessed slot within the main manifold and was sealed against a glass slide with machined polycarbonate clamps and an o-ring gasket. Each tissue reservoir was separated from the recirculation channel by a polypropylene insert over-molded onto a nylon mesh porous membrane. The membrane insert itself created a seal with the main manifold through the use of an elastomer o-ring. The plugs used to isolate tissue chambers (for culture in isolation) were machined from polycarbonate to create a seal via a fluoroelastomer o-ring.

The platform was connected to a peristaltic pump with a luer taper connector, with media flowing underneath through the connection channel. The media exited the channel into a reservoir, which also functions as a bubble trap. The reservoir was connected to the pump with a luer taper connector. PharmaMed pump tubing (Cole Parmer) routed the media back to the peristaltic pump (Cole Parmer) for recirculation.

To platform was contained within a 100 mm polystyrene dish that incorporated a secondary spacer between the dish and the lid to pass tubing in and out of the assembly without introducing gaps that would compromise sterility.

Software and equipment used for machined components include Solidworks for 3D design, Mastercam for toolpath generation, and a Haas OM2 3 axis milling machine for physical manufacturing. Polycarbonate and polysulfone materials were sourced from McMaster-Carr. For injection molding of porous membrane inserts, nylon meshes were sourced from Millipore, polypropylene pellets (Flint Hills Resources P9M7R-056) sourced from PolyOne Distribution, and molds were machined in aluminum using above fabrication equipment. Nylon mesh inserts were cut using a 40 W CO₂ laser cutter and inserted into the mold. Injection molding was performed on an AB-200 Semi-Automatic Plastic Injector (AB Machinery).

### Customized Microscope System

The customized microscope was assembled on an optical breadboard (12″ × 12″). The system includes a 2X plan apochromat objective lens that allows a lager field of view, a CMOS monochromatic camera, and exchangeable LED light sources. The camera is mounted vertically on a motorized optical rail that enables focus of different horizontal plains of the tissues with enhanced precision. The LED light source provides either a white light or a light with a specific wavelength when coupled with an optical filter allowing bright-field or fluorescent imaging. All optomechanical components were obtained from Thorlabs, while the objective lens was purchased from Edmund Optics.

### Sterility Assay

The platform was incubated for 4 weeks, at 25° C., with Soybean casein digest medium (SCDM), an aerobic bacteria and fungi specific medium. After the incubation period, any changes in the medium turbidity and the presence of microorganisms were assessed.

### Cell Culture

Human iPS cells were obtained through material transfer agreements from B. Conklin, Gladstone Institute (WTC11 line), maintained in mTeSR™1 medium (STEMCELL Technologies), supplemented with 1% penicillin/streptomycin, changed on a daily basis, on 1:60 growth-factor-reduced Matrigel (Corning) and passaged when 85-90 % confluent using 0.5 mm EDTA (Invitrogen). For the first 24 hours after passaging, the culture medium was supplemented with 5 µm Y-27632 dihydrochloride (Tocris).

Human mesenchymal stem cells (MSCs) were isolated from commercially obtained fresh bone marrow aspirates (Cambrex) by attachment to the plastic surface, as previously described. Cells were expanded to the fourth passage in mesenchymal stem cell medium consisting of high glucose Dulbecco’s modified Eagle’s medium (DMEM; Thermo Fisher Scientific) supplemented with 10% fetal bovine serum (FBS; Thermo Fisher Scientific), 1% penicillin-streptomycin (Life Technologies), and 0.1 ng/ml bFGF (Life Technologies).

The metastatic SK-N-MC (HTB-10) and non-metastatic RD-ES (HTB-166) ES cell lines were purchased from the American Type Culture Collection (ATCC). SK-N-MC cells were cultured in Eagle’s Minimum Essential Medium (EMEM; ATCC) and RD-ES cells were cultured in RPMI-1640 Medium (ATCC), according to the manufacturer’s specifications. Both culture media were supplemented with 10% FBS and 1 % penicillin/streptomycin.

All cells were maintained at 37° C. and 5% CO₂ in Heracell 150 incubators (Thermo Fisher Scientific). The cultures were maintained with 2 ml of medium per 10 cm² of surface area and were routinely checked for mycoplasma contamination using a MycoAlert Plus Kit (Lonza). Pluripotent cells were routinely checked for expression of pluripotent markers.

### GFP-Luciferase Transduction and Cell Sorting

A LentiSuite for HIV-based system (System Biosciences) was used according to the manufacturer’s instructions to generate stable CMV-GFP-T2A-Luciferase vector expressing ES (SK-N-MC and RD-ES). Briefly, HEK-293T (CRL-3216) cells were transfected with lentiviral and the GFP-Luciferase vector of interest, viral particles were purified and concentrated using a PEG-it Virus Precipitation Solution (System Biosciences). Cancer cell lines were transduced with the virus at MOI = 10 using Lipofectamine 3000 reagent (Thermo Fisher Scientific), according to the manufacturer’s protocols. GFP⁺ transduced cancer cells were selected and sorted for using an Influx cell sorter (BD Biosciences) in collaboration with the Columbia Center for Translational Immunology (CCTI) Flow Cytometry Core at Columbia University Irving Medical Center.

### Bone Matrix Scaffolds

Decellularized bone scaffolds were generated using a previously established protocol ⁵⁰ and cut into 2 mm thick axial sections. Sections to fabricate scaffolds were cleaned under high-pressure water beam, dried, and machined using a standard two-flute endmill to the final geometry of 6 mm × 3 mm × 1 mm (length x depth x thickness). To remove cellular material, the scaffolds were subjected to serial washes in 0.1 % EDTA in phosphate-buffered saline (PBS; Santa Cruz Biotechnology), 0.1 % EDTA in 10 mm Tris, and 0.5% SDS in 10 mm Tris, and a solution of 100 U/mL DNase and 1 U/mL RNase in 10 mM Tris buffer. Scaffolds were thoroughly rinsed in deionized water and freeze-dried. The scaffolds were selected within the density range of 0.37-0.45 mg/mm³ where sterilized overnight in 70 % ethanol and conditioned in mesenchymal stem cell medium overnight before seeding with cells. To monitor the effectiveness of the decellularization protocol, DNA content of the bone before and after decellularization was quantified using Quant-iT™ PicoGreen™ dsDNA Assay Kit (Thermo Fisher Scientific) following the manufacturer’s protocol.

### Tissue Engineered ES Tumors

Using an established protocol, expanded MSCs were seeded into the bone matrix scaffolds at a concentration of 10⁶ cells per scaffold, using 40 µL of medium. The cells were allowed to attach for 2 hours, and then supplemented with additional mesenchymal stem cell medium overnight. After 24 hours, osteogenic differentiation was initiated by addition of low glucose DMEM supplemented with 1 µm dexamethasone(Sigma-Aldrich), 10 mm β-glycerophosphate (SigmaAldrich), and 50 µm L-scorbic acid-2-phosphate (Sigma Aldrich). Each scaffold was incubated in 4 mL of osteogenic media, with media changes 3 times a week, for 3 weeks, allowing MSCs to differentiate into functional, maturing osteoblasts.

Two weeks following the initiation of osteogenic differentiation, aggregates of ES tumor cells were prepared as described previously, using 0.3 × 10⁶ cells per aggregate. After 1 week of culture, corresponding to the end of bone tissue culture (3 weeks), the primary ES cell aggregates were placed into the engineered bone constructs (3 aggregates per construct, placed apart of each other). Tumor models were established for two different types of primary ES cells: non-metastatic (RD-ES) and metastatic (SK-N-MC). Tissue engineered RD-ES and SK-N-MC tumors were cultured in the RPMI and EMEM media, respectively, supplemented with 10% FBS and 1% penicillin/streptomycin. Bone constructs cultured without tumor cell aggregates (TE-bone) in RPMI and EMEM media were used as controls.

Upon maturation, bone tumors in the platform were exposed to a 1:1 mixture of the bone tumor and cardiac tissue media, supplemented by 12 µM linsitinib in the treated groups. Two experimental conditions were systematically compared: isolated culture (no communication between the tissue chambers) and integrated culture (tissue chambers connected by microfluidic perfusion).

### Cardiac Differentiation of Human iPS Cells

Using a previously established protocol, cardiac differentiation of human iPS cells was initiated in 90% confluent cell monolayers by replacing the mTeSR™1 medium with CDM3 (chemically defined) medium with 3 components: RPMI Medium 1640 (1X, Gibco), 500 µg/mL of recombinant human albumin (Sigma-Aldrich) and 213 µg/mL of L-Ascorbic Acid 2-phosphate (Sigma-Aldrich)), supplemented with 1% penicillin/streptomycin.⁵⁶ Medium was changed every 48 hours. For the first 48 hours, medium was supplemented with 3 µm of glycogen synthase kinase 3-b inhibitor CHIR99021 (Tocris). On day 2, the culture was switched to CDM3 medium supplemented with 2 µm of the Wnt inhibitor Wnt-C59 (Tocris). After day 4 of differentiation, medium was changed to CDM3 with no supplements. Contracting cells were noted around day 10, when medium was changed to RPMI 1640 supplemented the with B-27™ (50X; Gibco) and were used in experiments without selection for cardiomyocytes.

### Tissue Engineered Cardiac Muscle

Using a methodology established in our previous studies, cardiac tissues were formed between two elastic pillars (1 mm in diameter, 9 mm in length, 6 mm axis-to-axis distance) that were over-molded onto a polycarbonate support frame. The pillars were formed using Delrin (polyoxymethylene) molds fabricated by CNC machining. PDMS was centrifugal casted at 400 relative centrifugal force (RCF) for 5 minutes through the polycarbonate support structures inserted into the molds. After centrifugation, PDMS was cured in an oven at60° C. for 1 hour and used at a 10:1 ratio of silicone elastomer base/curing agent. The resulting component pair of pillars to support the formation of one tissue, was inserted into the platform chamber by press-fitting. An array of 6 reservoirs accommodates formation of 6 individual pillar/tissue modules.

Human iPS cell-derived cardiomyocytes at day 13 of differentiation were combined with normal human dermal fibroblasts (NHDF; Lonza) at a ratio of 75% human iPS-derived cardiomyocytes and 25 % NHDF, for a total of 1 million cells per tissue. The hydrogel was formed by mixing 33 mg/mL of human fibrinogen (Sigma-Aldrich) with 25 U/mL of human thrombin (Sigma-Aldrich), at an 84:16 ratio. The cell suspension in hydrogel was dispensed into each well and allowed to polymerize around the pillars at 37° C. for 15 minutes before adding RPMI Medium 1640 supplemented with B-27™ containing 0.2 mg/ml aprotinin (Sigma-Aldrich).

Tissues were formed by inserting the pillars into a formation reservoir (9 mm length × 3.2 mm width × 4.3 mm depth) and can be filled with 100 µL of cell suspension in hydrogel. Hydrogel compaction caused passive tension of the tissues stretched between the two pillars, inducing elongation and alignment. Medium was changed every other day and supplemented with 0.2 mg/ml aprotinin for the first 7 days. Cardiac tissues were transferred into the platform chambers and cultured in either isolation or integrated by perfusion.

### Mathematical Model of Linsitinib Transport in the Platform

To evaluate drug transport in the blank platform, we performed computational fluid dynamics using a simultaneous finite volume solver (CoBi) that solves complex mass (continuity), momentum, energy, and drug conservation equations in two-dimensional discretization with heterogeneous properties (Equations 1-3)

The transport equations account for convection, diffusion, fluid-solid interaction, electrostatic drift and interfacial friction

$$\frac{\partial P}{\partial t} + ∇ \left(ρ \vec{v}\right) \qquad \text{(1)}$$

$$ρ \left(\frac{\partial \vec{V}}{\partial t} + \vec{v} ⋅ ∇ v\right) = ∇ P + μ \text{} {∇}^{2} \vec{v} + \vec{F} \qquad \text{(2)}$$

$$\frac{\partial C}{\partial t} = ∇ ⋅ \left(D ∇ C + \vec{v} C\right) + S \qquad \text{(3)}$$

where P is the pressure, t is time, p is the fluid density, v̅ is the bulk fluid velocity, µ is the fluid viscosity, F is the additional body force per unit mass, C is linsitinib concentration, D is the linsitinib diffusivity, and S is the source term. CoBi also has built-in modules to assign hydrodynamics (pressure, volumetric flux, and porous medium) and diffusion (partition coefficients, permeability, and diffusivity) properties.

Transwell membrane porosity was calculated by definition:

$$P o r o s i t y = \frac{{V}_{v o i d}}{{V}_{T o t a l}} \qquad \text{(4)}$$

where V_{void}is the void volume, and V_{Total} is the total membrane volume. Using manufactirer’s information for the total surface area, pore density, and pore size in the membrane, its porosity was calculated to 5%.

The Polson equation (Equation 5) was used to predict the diffusion coefficient:

$$D = \frac{9.4 \times {10}^{- 15} T}{μ M {W}^{\frac{1}{3}}} \qquad \text{(5)}$$

where the parameters are dynamic viscosity (µ) at absolute temperature (T), and molecular weight (MW). Linsitinib diffusion in media in media was calculated to 4.4×10⁻¹⁰ m²/s.

### Estimation of Linsitinib Absorption and Diffusive Transport

Fluorescein isothiocyanate (FITC, 10 mM in DMSO; Sigma Aldrich) was circulated for the integrated platform to determine potential hydrophobic small molecule absorption, given its physical and chemical properties. FITC was added at a concentration of 10 µM to 8 mL of 1:1 bone tumor/cardiac mixed media and introduced into the platform. The control was the FITC-containing media in a standard 24-multiwell plate (Corning). Aliquots from the reservoir, bone tumor, and cardiac tissue chambers were taken at 12, 24, 48 and 72 hours and measured for fluorescent signal using a spectrophotometer (Biotek).

Measurements of FITC concentrations were used to estimate the spread of linsitinib within the platform via diffusive circulating transport. Four independent platforms were filled with 8 mL of 1:1 mixed media each, after which 10 µM of FITC was injected into the reservoir. The platforms were connected to the peristaltic pumps run at 100 rpm to generate physiologically relevant fluid flow rate and shear stress. Aliquots were taken from the reservoir, bone and cardiac chambers at 0, 2, 4, 6, and 12 hours post injection, and assayed for fluorescence signal on a spectrophotometer (Biotek).

### Drug Treatments

Cardiac tissues were studied using caffeine (50 mM in water; Sigma-Aldrich), amiodarone hydrochloride (2.418 µM in DMSO; Sigma-Aldrich), isoproterenol hydrochloride (a series of drug concentrations in water; Sigma-Aldrich) or doxorubicin hydrochloride (1 µM in DMSO; Sigma-Aldrich), all diluted in RPMI Medium 1640 supplemented with B-27™. Response to isoproterenol was analyzed 10 minutes after exposure to 1 µm isoproterenol hydrochloride, diluted in RPMI Medium 1630 supplemented with B27™. ES bone tumor cell lines and tissues were studied using either doxorubicin hydrochloride (10 mM in water; Sigma-Aldrich), linsitinib (OSI-906) (10 mM in DMSO; Santa Cruz Biotechnology), all diluted in either non-metastastic media (RPMI Medium 1640, 10% FBS, 1% PenStrep) or metastatic media (EMEM, 10% FBS, 1% PenStrep). Specifically, linsitinib was dissolved at a 10 mM concentration in DMSO (Corning) and mixed in with the respective cell medium at a 12 µm concentration unless otherwise noted. Vehicle treatments involved just the addition of DMSO at identical volumes as a control. Tissues were randomly assigned to experimental groups. Medium was changed every day.

### Histology, Immunofluorescence, and Microscopy

Tissue samples were washed in PBS, fixed in 10% formalin at room temperature for 24 hours, and decalcified for 24 hours with Immunocal solution (Decal Chemical Corp.). Samples were then dehydrated in graded ethanol solutions, paraffin embedded, and sectioned to 5-µm thick.

For immunohistochemistry, tissue sections were deparaffinized with CitriSolv (Thermo Fisher Scientific) and rehydrated with graded ethanol washes. Antigen retrieval was performed by incubation in citrate buffer (pH 6) at 90° C. for 30 minutes, while endogenous peroxidase activity was blocked with 3% H₂O₂. After washing with PBS, sections were blocked with horse serum (Vector Labs) and stained with primary antibodies overnight in a humidified environment. The primary antibodies used were polyclonal rabbit IgG to CD99 (1:500; ab108297), polyclonal rabbit IgG to Ki67 (1:100; ab15580), polyclonal rabbit IgG to osteopontin (1:500; ab1870), and polyclonal rabbit IgG to bone sialoprotein 2 (1:500, ab1854).

After washing with PBS, samples were incubated with anti-rabbit secondary antibodies for 1 hour at 25° C. and developed as described previously (Vector Laboratories) and counterstained with Hematoxylin QS (Vector Labs). The images of histological sections were obtained by digitizing the tissue sections using the Olympus dotSlide 2.4 digital virtual microscopy system (Olympus) at a resolution of 0.32 µm.

To assess apoptosis, paraffin embedded tissue sections were first deparaffinized with CitriSolv, rehydrated with graded series of ethanol washes, and then stained with a Click-iT® TUNEL Alexa Fluor® imaging assay (Thermo Fisher Scientific). Following nuclear counterstaining with DAPI (Life Technologies), the TUNEL labelled slides were imaged with an IX81 inverted fluorescent microscope (Olympus) and a Pike F032B camera (ALLIED Vision), using NIS-Elements AR software, and processed using ImageJ (NIH). Four representative images per condition were then analyzed using the previously developed automatic TUNEL cell counter plugin for ImageJ to quantify DAPI⁺ cells and TUNEL⁺ cells.

To view the transduced fluorescent bone tumor aggregates in situ, the TE-ES samples were captured using a Nikon A1 scanning confocal microscope on an Eclipse Ti microscope stand (Nikon Instruments, Melville, NY) using a 10x/0.3 Plan Fluor (Nikon) objective. The confocal pinhole was set at 1 Airy unit, to produce an optical section of approximately 17 µm. GFP was excited at 488 nm and emission was collected from 500-550 nm. Z series were collected through the depth of the tissue section and maximum projections renderings were generated using NIS Elements software (Nikon). Images were collected in the Confocal and Specialized Microscopy Shared Resource of the Herbert Irving Comprehensive Cancer Center.

### Quantitative Real-time PCR

Total RNA was isolated using Trizol (Life Technologies), following the manufacturer’s instructions. RNA preparations (2 µg) were treated with a High-Capacity cDNA Reverse Transcription Kit (Applied Biosystems) to generate cDNA. Quantitative real-time PCR was performed using Fast SYBR™ Green Master Mix (Applied Biosystems). mRNA expression levels were quantified applying the ΔCt method, ΔCt = (Ct of gene of interest - Ct of β-Actin). Primer sequences were those that have been previously reported.

### Contractility Videos

To measure the cardiac contractility online, we took contractility videos of the tissues that were analyzed using the native MATLAB code we previously developed. Tissue contractility was measured by tracking the change in tissue area as a function of time. Acquired video frames were inverted and an automated intensity threshold was used to identify cell location in the video frame. First, a baseline time point in the video corresponding to a relaxed tissue state was selected. Absolute differences in cell area from the baseline frame were then calculated to create a time course of cell area changes over time. The resulting time courses were analyzed using a native MATLAB (MATHWorks) automated peak finding algorithm to determine locations of maximum cell contractions in the time profiles. Beat period lengths were determined from the length of time between the pairs of local maxima, and the beat frequencies were determined by inverting beat periods. The rate of proarrhythmic events was calculated by the ratio of the number of proarrhythmic events over the total number of beats.

### Cell Viability

Cell viability was analyzed using a previously established protocol.⁵⁹ Cancer cell viability was measured for GFP-Luciferase labelled cancer cells using ONE-Glo luciferase substrate that was prepared according to manufacturer’s protocol (Promega). Samples were collected following 3, 7, and 21 days cycles of linsitinib treatment. Where noted, longitudinal cell viability was also assessed using luminescence, though at the cost of signal strength. Briefly, in vivo grade VivoGlo™ Luciferin (Promega) was made at a 200x stock concentration (30 mg/ml) in water, added to sample culture media at a 1:200 dilution, and scanned using a spectrophotometer (Biotek). Some of the IC₅₀ values were determined using cell viability data generated using an MTT assay (RealTime-Glo™ MT Cell Viability Assay, Promega) that were analyzed according to manufacturer’s protocol.

### Secreted Protein Quantification

Proteomic analysis of secreted IGF-BPs was performed using supernatants isolated from RD-ES and SK-N-MC monolayers as well as both non-metastatic and metastatic TE-ES samples. A Pierce™ BCA Protein Assay Kit (ThermoFisher) was used to quantify protein amounts across the samples, after which equivalent amounts were loaded and processed onto a Human IGF Signaling Array (RayBiotech) according to manufacturer’s instructions. The samples were shipped to RayBiotech for quantification.

In order to confirm linsitinib mechanism of action in ES cells, both RD-ES and SK-N-MC monolayers were treated with 12 µM linsitinib for 6 hours, lysed, measured for protein quantity using a Pierce™ BCA Protein Assay Kit (ThermoFisher), and loaded equally onto a Human Phospho-IGF1R ELISA (RayBiotech) to semi-quantitatively determine phosphorylated levels of the IGF-1 receptor, according to manufacturer’s instructions. Osteocalcin (OCN), osteopontin (OPN), and lactic acid dehydrogenase (LDH) secreted levels were all measured using a similar approach. Supernatants were isolated from controls and drug treated TE-ES and equal amounts were used in each assay according to the manufacturer’s instructions. For OCN a Human Osteocalcin Quantikine^{R} ELISA (R&D Systems) was used, while for OPN it was a Human Osteopontin Quantikine^{R} ELISA (R&D Systems). LDH secretion was determined using a Lactate Dehydrogenase Assay Kit (Colorimetric; Abcam).

### Statistical Methods

Data were analyzed in Excel (Microsoft) and graphed in Prism (GraphPad). Data are presented as mean ± s.e.m. Differences between experimental groups were analyzed by unpaired, two-tailed Student’s t-test or two-way ANOVA with Bonferroni post-test. Significant differences defined by P < 0.05 for all statistical methods unless otherwise noted. No blinding or randomization was used.

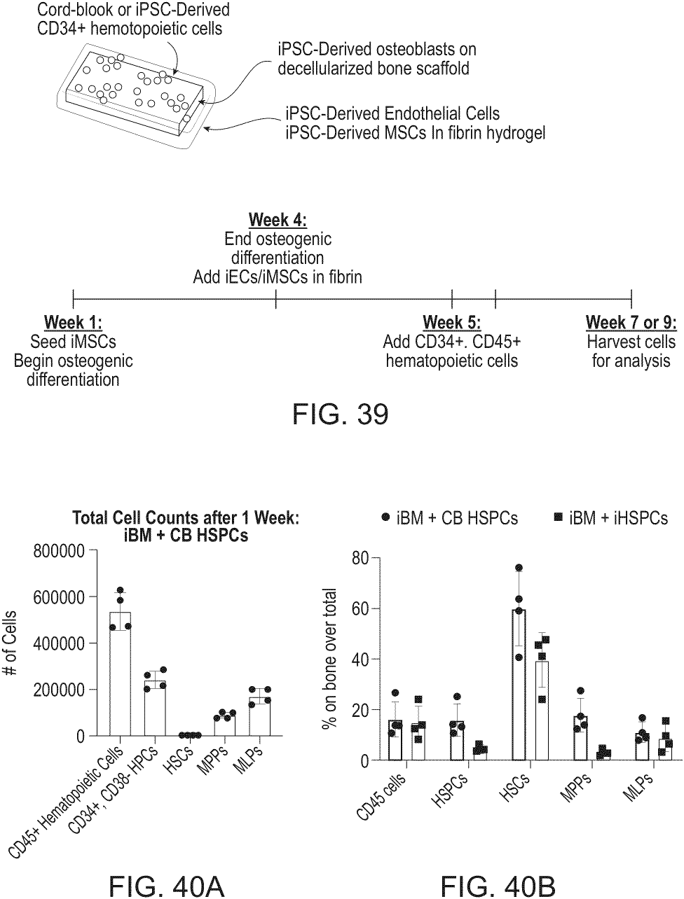

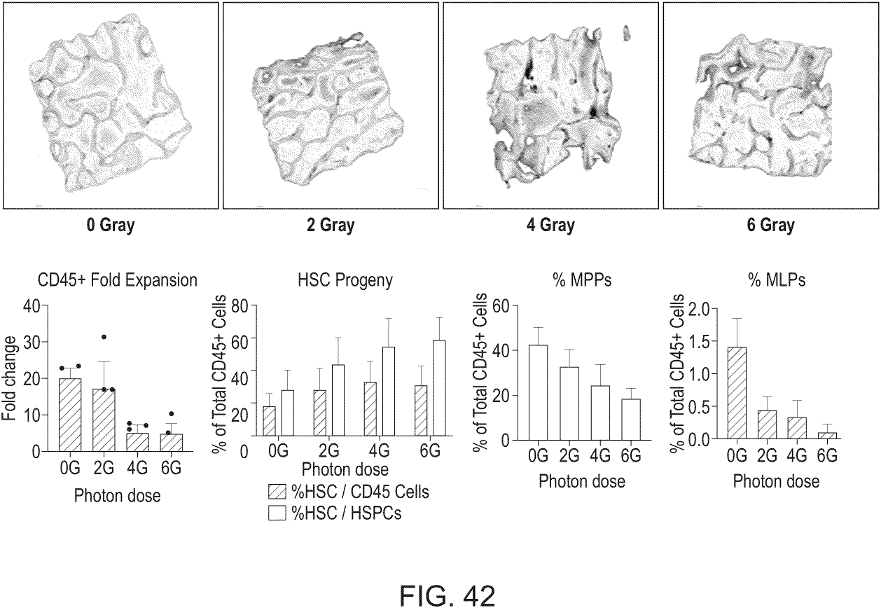

In another aspect, present disclosure relates to an engineered bone marrow system to study a variety of human diseases, toxicity responses, and immune functions in vitro. Derived from multiple cell sources, we have engineered an all induced pluripotent stem cell (iPSC) bone marrow model comprised of osteoblasts, mesenchymal stem/stromal cells, endothelial cells, and hematopoietic stem and progenitor cells (HSPCs). Using decellularized bone, iPSC-derived cells, and fibrin hydrogels in combination, we are able to study the progression, proliferation, and differentiation of hematopoietic cells in an in vitro setting. In addition to iPSC-derived sources, we are able to create the same bone marrow model using primary human samples (see overview in FIG. 39) This model of bone marrow incorporates entirely iPSC-derived stromal cells (osteoblasts, endothelial cells, and mesenchymal stem/stromal cells), as well as a 3D, niche-like environment for the support of iPSC-derived HSPCs. Our engineered bone marrow model can fill the role of a patient-specific, controllable, and complex bone marrow niche to support HSPCs in vitro and also allow for modeling of genetic blood/immune disorders, as well as radiotoxicity as we have outlined in FIG. 42, for up to 28 days. Notably, the engineered bone marrow could also serve as a potency assay to evaluate the grafting potential of iPS derived HSCs to evaluate their utility for regenerative medicine/patient cell therapy. Currently people use a rodent model for this and they inject the developed HSCs into the rodent to see if the engraft into the bone, thereby determining the HSCs as functional. A humanized way to do this may be valuable and would be enabled with this the techology described herein.

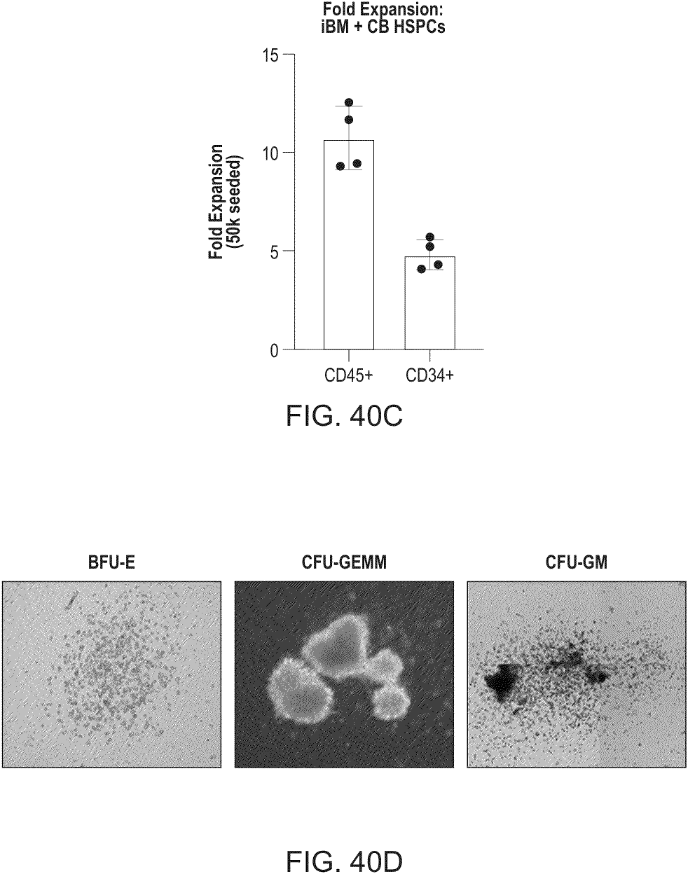

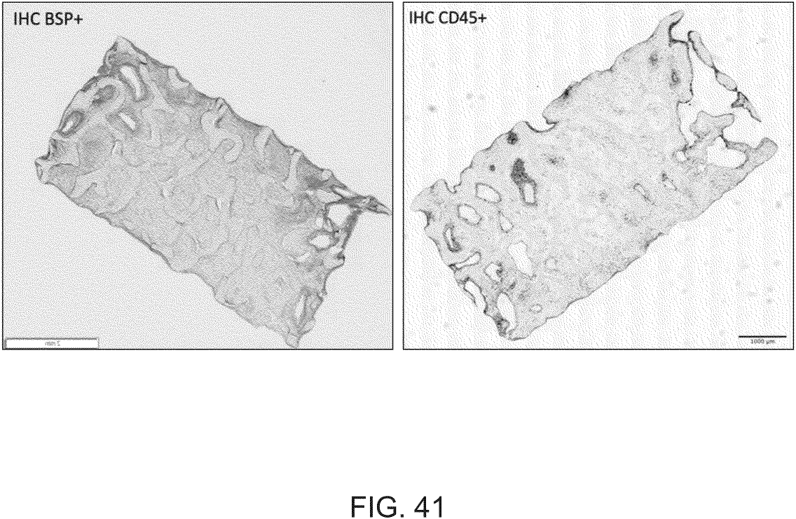

Referring to FIG. 39, iPSC-derived stromal cell types include osteoblasts, mesenchymal stem/stromal cells, endothelial cells & hematopoietic cell types include iPSC-derived HSCs, myeloid progenitors, lymphoid progenitors, mature macrophages/monocytes, mature B cells, immature T cells, megakaryocytes, among others). In FIGS. 40A-D, validation of model after 1 week of culture is shown. FIG. 40A illustrates total cell counts with various sub-specifications of hematopoietic cells. FIG. 40B illustrates percentage of cells attached to the bone marrow niche compartment. FIG. 40C illustrates fold expansion of CD45+ and CD34+ cells with minimal cytokines. FIG. 40D illustrates colony-forming capacity of isolated cells post-culture. Referring to FIG. 41, bone marrow morphology with stains for osteoblasts (BSP) and hematopoietic cells (CD45) are depicted. Referring to FIG. 42, application of bone marrow niche to modeling radiotoxicity of human tissue in vitro is shown. Morphological features demonstrated in top panels 2 weeks after exposure to 0G, 2G, 4G, and 6G of radiation in pentachrome staining, as well as expansion/progenitor differentiation capacity after 2 weeks post-radiation exposure of hematopoietic cells.

The “organ on a chip” provies fast track for translation of tissue engineering. Mature tissues are maintained within their own special niches. We tested physiological responses to radiation and simulated microgravity. Themesurements of readation damge and responses to radioprotective agents. Individualized models using iPSC derived platforms were used. Drugs were dosed and radation according to predictd levels in space were tested. Then validation against pre-clincial modles (mice) and clinical outfcomes (astronauts) was attempted.

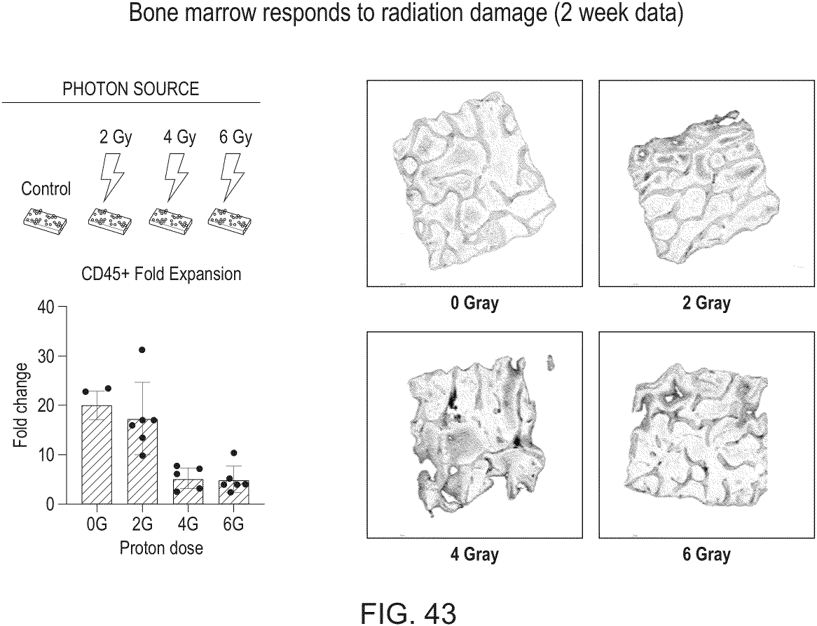

In the radiation studies, we included a control and photon sources at 2 Gy, 4 Gy and 6 Gy to bone marrow for two weeks. FIG. 43 shows images of the bone marrow response to the radiation compared to the control. After being matured in culture, the bone marrow is expressing the right molecular markers, contains cells that have clonal capacity, and displays hematopoiesis.

FIG. 43 shows a bar graph CD45+ fold expansion based on radiation dose is shown. As photon dose increased, fold change decreased showing an inversely propoporational relationship. Radiation is known to damage cells in native bone marrow, and this is what we also see for bioengineered bone marrow - damage reflected in decreased native ability of the cells to maintain themselves and profilerate.

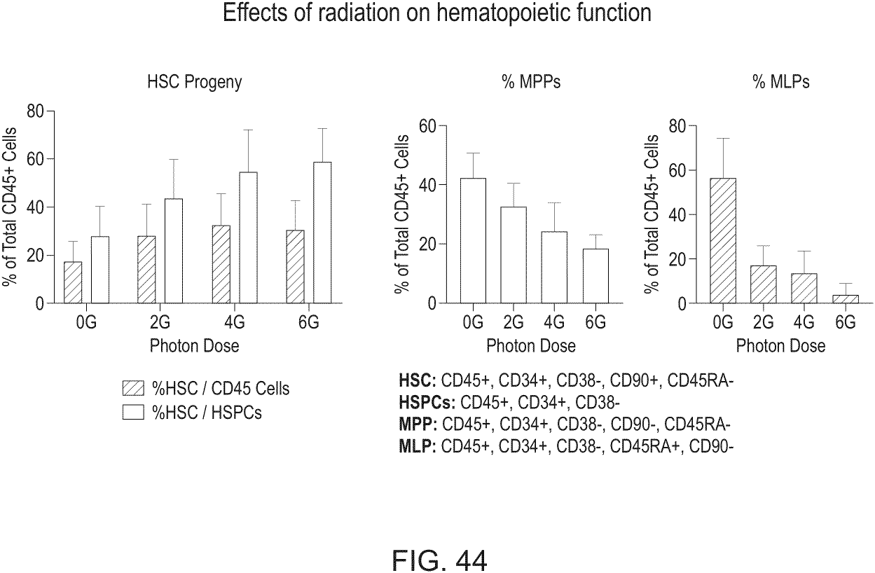

FIG. 44 shows the effects of radiation on hemoatopoietic function. Referring to FIG. 44, we see the effects of radiation on cell expansion.

What is claimed is:

1-29. (canceled)

30. A modular system for culturing systemic bioengineered tumor models, the system comprising:
a platform having a substantially planar body including a bottom surface and a top surface, the top surface defining at least one seat, and the bottom surface including a channel disposed along the platform body; and
a portable tissue chamber releasably mounted to the seat of the platform, the portable tissue chamber including an internal compartment configured to hold a tissue culture, the internal compartment being defined by a bottom surface of the portable tissue chamber, a plurality of sidewalls of the portable tissue chamber extending from the bottom surface of the portable tissue chamber, and an open top of the portable tissue chamber defined by the plurality of sidewalls of the portable tissue chamber, the bottom surface of the portable tissue chamber being formed at least in part by a permeable membrane positioned proximate the channel of the platform;
wherein the platform includes a first coupling member extending from the platform body, the portable tissue chamber includes a second coupling member releasably engaged to the first coupling member of the platform to secure the portable tissue chamber to the seat of the platform, and one or more of the first coupling member of the platform and the second coupling member of the portable tissue chamber are pivotable with respect to the other.

31. The system of claim 30, wherein the channel is in communication with the portable tissue chamber.

32. The system of claim 30, comprising an on-board pump disposed on the platform body, wherein the on-board pump is operatively connected to a reservoir, and the reservoir is configured to hold media.

33. The system of claim 32, comprising a first tubing operatively connected to the on-board pump, an entrance port disposed on the platform, a second tubing operatively connected to the on-board pump, and an exit port disposed on the platform.

34. The system of claim 32, wherein the reservoir is disposed on the platform body and the platform body includes a media entrance port and a media exit port.

35. The system of claim 30, wherein the channel of the platform body is operatively associated with the permeable membrane of the portable tissue chamber.

36. The system of claim 30, wherein the platform further comprises a media entrance port and a media exit port, each disposed on the top surface of the platform body.

37. The system of claim 30, including a plate disposed beneath the platform.

38. The system of claim 30, wherein the channel of the platform is configured to permit media flow through the system.

39. The system of claim 30, wherein the permeable membrane is selectively permeable to media flowing through the system.

40. The system of claim 30, comprising a plurality of portable tissue chambers mounted to a corresponding seat defined by the top surface of the platform body, wherein each portable tissue chamber contains a different tissue type.

41. The system of claim 30, comprising a plurality of portable tissue chambers mounted to a corresponding seat defined by the top surface of the platform body, wherein the plurality of modular tissue chambers includes at least a first portable tissue chamber, a second portable tissue chamber, a third portable tissue chamber, and a fourth portable tissue chamber.

42. The system of claim 41, wherein the first portable tissue chamber contains liver tissue, the second portable tissue chamber contains heart tissue, the third portable tissue chamber contains skin or lung tissue, and the fourth portable tissue chamber contains bone tissue.

43. The system of claim 30, comprising a plurality of portable tissue chambers mounted to a corresponding seat defined by the top surface of the platform body, wherein the plurality of portable tissue chambers includes at least a first portable tissue chamber and a second portable tissue chamber, and the first portable tissue chamber contains osteosarcoma cells and the second portable tissue chamber contains breast adenocarcinoma cells.

44. The system of claim 43, wherein the osteosarcoma and breast adenocarcinoma cells are derived from one patient.

45. The system of claim 30, wherein the media includes one or more of tumor cells and immune cells.

46. The system of claim 30, wherein the second coupling member of the portable tissue chamber extends from one or more sidewall of the plurality of sidewalls of the portable tissue chamber.

47. The system of claim 30, wherein the second coupling member of the portable tissue chamber extends from one or more sidewall of the plurality of sidewalls that is proximate an outer edge of the platform.

48. The system of claim 30, wherein the second coupling member of the portable tissue chamber is integrally formed on one or more sidewall of the plurality of sidewalls.

49. The system of claim 30, wherein the coupling member of the portable tissue chamber is configured to pivot with respect to the first coupling member of the platform.

50. A modular system for culturing systemic bioengineered tumor models, the system comprising:
a platform having a substantially planar body including a bottom surface and a top surface, the top surface defining at least one seat, and the bottom surface including a channel disposed along the platform body; and
a portable tissue chamber configured to releasably mount to the seat of the platform, the portable tissue chamber including an internal compartment configured to hold a tissue culture, the internal compartment being defined by a bottom surface of the portable tissue chamber, a plurality of sidewalls of the portable tissue chamber extending from the bottom surface of the portable tissue chamber, and an open top of the portable tissue chamber defined by the plurality of sidewalls of the portable tissue chamber, the bottom surface of the portable tissue chamber being formed at least in part by a permeable membrane configured to be positioned proximate the channel of the platform when the platform is mounted to the seat of the platform;
wherein the platform includes a first coupling member extending from the platform body, the portable tissue chamber includes a second coupling member extending from the portable tissue chamber, one or more of the first coupling member of the platform and the second coupling member of the portable tissue chamber are configured to be pivotable with respect to the other, and the first coupling member of the platform is configured to releasably engage the second coupling member of the portable tissue chamber to secure the portable tissue chamber to the seat of the platform when one or more of the first coupling member of the platform and the second coupling member of the portable tissue chamber pivots with respect to the other.

51. A portable tissue chamber configured for use in a modular system for culturing systemic bioengineered tumor models, the portable tissue chamber comprising:
an internal compartment configured to hold a tissue culture, the internal compartment being defined by a bottom surface of the portable tissue chamber, a plurality of sidewalls of the portable tissue chamber extending from the bottom surface of the portable tissue chamber, and an open top of the portable tissue chamber defined by the plurality of sidewalls of the portable tissue chamber, the bottom surface of the portable tissue chamber being configured to be secured in a mounted position within the modular system;
a coupling member configured to releasably secure the portable tissue chamber in the mounted position within the modular system, the coupling member extending from one or more sidewall of the plurality of sidewalls of the portable tissue chamber, and the coupling member being configured to be transitioned between a disengaged state and an engaged state; and
a permeable membrane forming at least a portion of the bottom surface of the portable tissue chamber,
wherein, when the coupling member is in the disengaged state, the bottom surface of the portable tissue chamber is not secured in the mounted position within the modular system and, when the coupling member is in the engaged state, the bottom surface of the portable tissue chamber is secured in the mounted position within the modular system.

52. The portable tissue chamber of claim 51, wherein the coupling member is integrally formed on the one or more sidewall of the plurality of sidewalls of the portable tissue chamber.

53. The portable tissue chamber of claim 51, wherein the coupling member is configured to pivot between the disengaged state and the engaged state.
